SEQUENCE LISTING (97 sequences)

SEQ ID NO: 1 - protein, 21 aa, synthetic construct
MALPVTALLLPLALLLHAARP

SEQ ID NO: 2 - protein, 18 aa, synthetic construct
MLRLLLALNLFPSIQVTG

SEQ ID NO: 3 - protein, 20 aa, synthetic construct
METDTLLLWVLLLWVPGSTG

SEQ ID NO: 4 - protein, 44 aa, synthetic construct
TTTPAPRPPTPAPTIASQPLSLRPEACRPAAGGAVHTRGLDFAC

SEQ ID NO: 5 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 119 aa, synthetic construct
EPKSCDKTHTCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 12 aa, synthetic construct
EPKSCDKTHTCP

SEQ ID NO: 8 - protein, 12 aa, synthetic construct
ESKYGPPCPPCP

SEQ ID NO: 9 - protein, 29 aa, synthetic construct
TTTPAPRPPTPAPTIASQPLSLRPEAFAC

SEQ ID NO: 10 - protein, 14 aa, synthetic construct
TTTPAPRPPTPFAC

SEQ ID NO: 11 - protein, 39 aa, synthetic construct
IEVMYPPPYLDNEKSNGTIIHVKGKHLCPSPLFPGPSKP

SEQ ID NO: 12 - protein, 39 aa, synthetic construct; identical to SEQ ID NO: 11

SEQ ID NO: 13 - protein, 26 aa, synthetic construct
KSNGTIIHVKGKHLCPSPLFPGPSKP

SEQ ID NO: 14 - protein, 16 aa, synthetic construct
GKHLCPSPLFPGPSKP

SEQ ID NO: 15 - protein, 15 aa, synthetic construct
GGGGSGGGGSGGGGS

SEQ ID NO: 16 - protein, 30 aa, synthetic construct
GGGGSGGGGSGGGGSGGGGSGGGGSGGGGS

SEQ ID NO: 17 - protein, 45 aa, synthetic construct
GGGGSGGGGSGGGGSGGGGSGGGGSGGGGSGGGGSGGGGSGGGGS

SEQ ID NO: 18 - protein, 60 aa, synthetic construct
GGGGSGGGGSGGGGSGGGGSGGGGSGGGGSGGGGSGGGGSGGGGSGGGGSGGGGSGGGGS

SEQ ID NO: 19 - protein, 75 aa, synthetic construct
GGGGSGGGGSGGGGSGGGGSGGGGSGGGGSGGGGSGGGGSGGGGSGGGGSGGGGSGGGGSGGGGSGGGGSGGGGS

SEQ ID NO: 20 - protein, 150 aa, synthetic construct
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 21 - protein, 225 aa, synthetic construct
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 22 - protein, 300 aa, synthetic construct
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 23 - protein, 25 aa, synthetic construct
DIYIWAPLAGTCGVLLLSLVITLYC

SEQ ID NO: 24 - protein, 28 aa, synthetic construct
DIISFFLALTSTALLFLLFFLTLRFSVV

SEQ ID NO: 25 - protein, 30 aa, synthetic construct
DFWVLVVVGGVLACYSLLVTVAFIIFWVRS

SEQ ID NO: 26 - protein, 29 aa, synthetic construct
FWVLVVVGGVLACYSLLVTVAFIIFWVRS

SEQ ID NO: 27 - protein, 27 aa, synthetic construct
FWVLVVVGGVLACYSLLVTVAFIIFWV

SEQ ID NO: 28 - protein, 25 aa, synthetic construct
DLIALVTSGALLAVLGITGYFLMNR

SEQ ID NO: 29 - protein, 25 aa, synthetic construct
DLLAALLALLAALLALLAALLARSK

SEQ ID NO: 30 - protein, 26 aa, synthetic construct
DMALIVLGGVAGLLLFIGLGIFFCVR

SEQ ID NO: 31 - protein, 25 aa, synthetic construct
DLCYILDAILFLYGIVLTLLYCRLK

SEQ ID NO: 32 - protein, 25 aa, synthetic construct
DLLLILLGVLAGVLATLAALLARSK

SEQ ID NO: 33 - protein, 25 aa, synthetic construct
DITLGLLVAGVLVLLVSLGVAIHLC

SEQ ID NO: 34 - protein, 25 aa, synthetic construct
DVSFCLVMVLLFAVDTGLYFSVKTN

SEQ ID NO: 35 - protein, 25 aa, synthetic construct
DVAAILGLGLVLGLLGPLAILLALY

SEQ ID NO: 36 - protein, 29 aa, synthetic construct
DVMSVATIVIVDICITGGLLLLVYYWSKN

SEQ ID NO: 37 - protein, 25 aa, synthetic construct
DLCYLLDGILFIYGVILTALFLRVK

SEQ ID NO: 38 - protein, 25 aa, synthetic construct
DGFLFAEIVSIFVLAVGVYFIAGQD

SEQ ID NO: 39 - protein, 26 aa, synthetic construct
DGIIVTDVIATLLLALGVFCFAGHET

SEQ ID NO: 40 - protein, 24 aa, synthetic construct
DVIGFRILLLKVAGFNLLMTLRLW

SEQ ID NO: 41 - protein, 27 aa, synthetic construct
DIIVAVVIATAVAAIVAAVVALIYCRK

SEQ ID NO: 42 - protein, 25 aa, synthetic construct
DVLFYLAVGIMFLVNTVLWVTIRKE

SEQ ID NO: 43 - protein, 25 aa, synthetic construct
DIIILVGTAVIAMFFWLLLVIILRT

SEQ ID NO: 44 - protein, 21 aa, synthetic construct
DLGWLCLLLLPIPLIVWVKRK

SEQ ID NO: 45 - protein, 25 aa, synthetic construct
DIAIYCIGVFLIACMVVTVILCRMK

SEQ ID NO: 46 - protein, 28 aa, synthetic construct
FACDIYIWAPLAGTCGVLLLSLVITLYC

SEQ ID NO: 47 - protein, 42 aa, synthetic construct
KRGRKKLLYIFKQPFMRPVQTTQEEDGCSCRFPEEEEGGCEL

SEQ ID NO: 48 - protein, 41 aa, synthetic construct
RSKRSRLLHSDYMNMTPRRPGPTRKHYQPYAPPRDFAAYRS

SEQ ID NO: 49 - protein, 42 aa, synthetic construct
ALYLLRRDQRLPPDAHKPPGGGSFRTPIQEEQADAHSTLAKI

SEQ ID NO: 50 - protein, 22 aa, synthetic construct
ERVQPLEENVGNAARPRFERNK

SEQ ID NO: 51 - protein, 34 aa, synthetic construct
KKKYSSSVHDPNGEYMFMRAVNTAKKSRLTDVTL

SEQ ID NO: 52 - protein, 48 aa, synthetic construct
QRRKYRSNKGESPVEPAEPCHYSCPREEEGSTIPIQEDYRKPEPACSP

SEQ ID NO: 53 - protein, 58 aa, synthetic construct
QLGLHIWQLRSQCMWPRETQLLLEVPPSTEDARSCQFPEEERGERSAEEKGRLGDLWV

SEQ ID NO: 54 - protein, 60 aa, synthetic construct
CVKRRKPRGDVVKVIVSVQRKRQEAEGEATVIEALQAPPDVTTVAVEETIPSFTGRSPNH

SEQ ID NO: 55 - protein, 48 aa, synthetic construct
KKYFFKKEVQQLSVSFSSLQIKALQNAVEKEVQAEDNIYIENSLYATD

SEQ ID NO: 56 - protein, 84 aa, synthetic construct
DIYIWAPLAGTCGVLLLSLVITLYCYKVGFFKRNLKEKMEAGRGVPNGIPAEDSEQLASGQEAGDPGCLKPLHEKDSESGGGKD

SEQ ID NO: 57 - protein, 141 aa, synthetic construct
DIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 58 - protein, 89 aa, synthetic construct
RSKRSRLLHSDYMNMTPRRPGPTRKHYQPYAPPRDFAAYRSQRRKYRSNKGESPVEPAEPCHYSCPREEEGSTIPIQEDYRKPEPACSP

SEQ ID NO: 59 - protein, 78 aa, synthetic construct
RSKRSRLLHSDYMNMTPRRPGPTRKHYQPYAPPRDFAAYRSRRDQRLPPDAHKPPGGGSFRTPIQEEQADAHSTLAKI

SEQ ID NO: 60 - protein, 83 aa, synthetic construct
KRGRKKLLYIFKQPFMRPVQTTQEEDGCSCRFPEEEEGGCELRSKRSRLLHSDYMNMTPRRPGPTRKHYQPYAPPRDFAAYRS

SEQ ID NO: 61 - protein, 80 aa, synthetic construct
RSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 62 - protein, 75 aa, synthetic construct
RSKRSRLLHSDYMNMTPRRPGPTRKHYQPYAPPRDFAAYRSKKKYSSSVHDPNGEYMFMRAVNTAKKSRLTDVTL

SEQ ID NO: 63 - protein, 41 aa, synthetic construct
RSKRSRGGHSDYMNMTPRRPGPTRKHYQPYAPPRDFAAYRS

SEQ ID NO: 64 - protein, 83 aa, synthetic construct
RSKRSRGGHSDYMNMTPRRPGPTRKHYQPYAPPRDFAAYRSKRGRKKLLYIFKQPFMRPVQTTQEEDGCSCRFPEEEEGGCEL

SEQ ID NO: 65 - protein, 23 aa, synthetic construct
CARPRRSPAQEDGKVYINMPGRG

SEQ ID NO: 66 - protein, 61 aa, synthetic construct
NRRRRRERRDLFTESWDTQKAPNNYRSPISTSQPTNQSMDDTREDIYVNYPTFSRRPKTRV

SEQ ID NO: 67 - protein, 45 aa, synthetic construct
GSTVYYQGKCLTWKGPRRQLPAVVPAPLPPPCGSSAHLLPPVPGG

SEQ ID NO: 68 - protein, 63 aa, synthetic construct
WWGDIWWKTMMELRSLDTQKATCHLQQVTDLPWTSVSSPVEREILYHTVARTKISDDDDEHTL

SEQ ID NO: 69 - protein, 28 aa, synthetic construct
GKPIPNPLLGLDSTGKPIPNPLLGLDST

SEQ ID NO: 70 - protein, 6 aa, synthetic construct
HHHHHH

SEQ ID NO: 71 - protein, 16 aa, synthetic construct
DYKDDDDKDYKDDDDK

SEQ ID NO: 72 - protein, 27 aa, synthetic construct
YPYDVPDYAYPYDVPDYAYPYDVPDYA

SEQ ID NO: 73 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 19 aa, synthetic construct
ATNFSLLKQAGDVEENPGP

SEQ ID NO: 75 - protein, 270 aa, synthetic construct
MARFALTVVRHGETRFNKEKIIQGQGVDEPLSETGFKQAAAAGIFLNNVKFTHAFSSDLMRTKQTMHGILERSKFCKDMTVKYDSRLRERKYGVVEGKALSELRAMAKAAREECPVFTPPGGETLDQVKMRGIDFFEFLCQLILKEADQKEQFSQGSPSNCLETSLAEIFPLGKNHSSKVNSDSGIPGLAASVLVVSHGAYMRSLFDYFLTDLKCSLPATLSRSELMSVTPNTGMSLFIINFEEGREVKPTVQCICMNLQDHLNGLTETR

SEQ ID NO: 76 - protein, 492 aa, synthetic construct
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 77 - protein, 430 aa, synthetic construct
MALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKVMADRIIGMRTQLVSNLKKEGSTHNWQHITDQIGMFCFTGLKPEQVERLIKEFSIYMTKDGRISVAGVTSSNVGYLAHAIHQVTK

SEQ ID NO: 78 - protein, 332 aa, synthetic construct
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 79 - protein, 456 aa, synthetic construct
MRLARLLRGAALAGPGPGLRAAGFSRSFSSDSGSSPASERGVPGQVDFYARFSPSPLSMKQFLDFGSVNACEKTSFMFLRQELPVRLANIMKEISLLPDNLLRTPSVQLVQSWYIQSLQELLDFKDKSAEDAKAIYERPRRTWLQVSSLCCMACKMIFTDTVIRIRNRHNDVIPTMAQGVIEYKESFGVDPVTSQNVQYFLDRFYMSRISIRMLLNQHSLLFGGKGKGSPSHRKHIGSINPNCNVLEVIKDGYENARRLCDLYYINSPELELEELNAKSPGQPIQVVYVPSHLYHMVFELFKNAMRATMEHHANRGVYPPIQVHVTLGNEDLTVKMSDRGGGVPLRKIDRLFNYMYSTAPRPRVETSRAVPLAGFGYGLPISRLYAQYFQGDLKLYSLEGYGTDAVIYIKALSTDSIERLPVYNKAAWKHYNTNHEADDWCVPSREPKDMTTFRSA

SEQ ID NO: 80 - protein, 412 aa, synthetic construct
MSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 81 - protein, 225 aa, synthetic construct
MVSHSELRKLFYSADAVCFDVDSTVIREEGIDELAKICGVEDAVSEMTRRAMGGAVPFKAALTERLALIQPSREQVQRLIAEQPPHLTPGIRELVSRLQERNVQVFLISGGFRSIVEHVASKLNIPATNVFANRLKFYFNGEYAGFDETQPTAESGGKGKVIKLLKEKFHFKKIIMIGDGATDMEACPPADAFIGFGGNVIRQQVKDNAKWYITDFVELLGELEE

SEQ ID NO: 82 - protein, 405 aa, synthetic construct
MQEKDASSQGFLPHFQHFATQAIHVGQDPEQWTSRAVVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHS

SEQ ID NO: 83 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 84 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 85 - protein, 243 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 86 - protein, 487 aa, synthetic construct
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 483 aa, synthetic construct
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 18 aa, synthetic construct
EGRGSLLTCGDVEENPGP

SEQ ID NO: 89 - protein, 20 aa, synthetic construct
QCTNYALLKLAGDVESNPGP

SEQ ID NO: 90 - protein, 23 aa, synthetic construct
AVKQTLNFDLLKLAGDVESNPGP

SEQ ID NO: 91 - DNA, 463 bp, synthetic construct
  source 1..463: mol_type = other DNA
gagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 92 - protein, 1230 aa, synthetic construct
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKVMADRIIGMRTQLVSNLKKEGSTHNWQHITDQIGMFCFTGLKPEQVERLIKEFSIYMTKDGRISVAGVTSSNVGYLAHAIHQVTKGSGEGRGSLLTCGDVEENPGPMARFALTVVRHGETRFNKEKIIQGQGVDEPLSETGFKQAAAAGIFLNNVKFTHAFSSDLMRTKQTMHGILERSKFCKDMTVKYDSRLRERKYGVVEGKALSELRAMAKAAREECPVFTPPGGETLDQVKMRGIDFFEFLCQLILKEADQKEQFSQGSPSNCLETSLAEIFPLGKNHSSKVNSDSGIPGLAASVLVVSHGAYMRSLFDYFLTDLKCSLPATLSRSELMSVTPNTGMSLFIINFEEGREVKPTVQCICMNLQDHLNGLTETR

SEQ ID NO: 93 - protein, 248 aa, synthetic construct
DIVMTQSPLSQPVTPGEPASISCRSSQSLLHRYGYNSLHWYLQKPGQSPQLLIYLGSNRASGVPDRFSGSGSGTDFTLKVSRVEAEDVGVYYCMQALQTPYTFGQGTKLEIKGSTSGSGKPGSGEGSTKGQVQLQESGPGLVKPSGTLSLTCAVSGGSISSSNWWSWVRQPPGKGLEWLGEISHSGITNYNPSLKSRVTISVDKSKNHFSLKLNSVTAADTAVYYCTKKWELLAFDFWGQGTMVTVSS

SEQ ID NO: 94 - protein, 462 aa, synthetic construct
MALPVTALLLPLALLLHAARPDIVMTQSPLSQPVTPGEPASISCRSSQSLLHRYGYNSLHWYLQKPGQSPQLLIYLGSNRASGVPDRFSGSGSGTDFTLKVSRVEAEDVGVYYCMQALQTPYTFGQGTKLEIKGSTSGSGKPGSGEGSTKGQVQLQESGPGLVKPSGTLSLTCAVSGGSISSSNWWSWVRQPPGKGLEWLGEISHSGITNYNPSLKSRVTISVDKSKNHFSLKLNSVTAADTAVYYCTKKWELLAFDFWGQGTMVTVSSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 754 aa, synthetic construct
MALPVTALLLPLALLLHAARPDIVMTQSPLSQPVTPGEPASISCRSSQSLLHRYGYNSLHWYLQKPGQSPQLLIYLGSNRASGVPDRFSGSGSGTDFTLKVSRVEAEDVGVYYCMQALQTPYTFGQGTKLEIKGSTSGSGKPGSGEGSTKGQVQLQESGPGLVKPSGTLSLTCAVSGGSISSSNWWSWVRQPPGKGLEWLGEISHSGITNYNPSLKSRVTISVDKSKNHFSLKLNSVTAADTAVYYCTKKWELLAFDFWGQGTMVTVSSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMARFALTVVRHGETRFNKEKIIQGQGVDEPLSETGFKQAAAAGIFLNNVKFTHAFSSDLMRTKQTMHGILERSKFCKDMTVKYDSRLRERKYGVVEGKALSELRAMAKAAREECPVFTPPGGETLDQVKMRGIDFFEFLCQLILKEADQKEQFSQGSPSNCLETSLAEIFPLGKNHSSKVNSDSGIPGLAASVLVVSHGAYMRSLFDYFLTDLKCSLPATLSRSELMSVTPNTGMSLFIINFEEGREVKPTVQCICMNLQDHLNGLTETR

SEQ ID NO: 96 - protein, 914 aa, synthetic construct
MALPVTALLLPLALLLHAARPDIVMTQSPLSQPVTPGEPASISCRSSQSLLHRYGYNSLHWYLQKPGQSPQLLIYLGSNRASGVPDRFSGSGSGTDFTLKVSRVEAEDVGVYYCMQALQTPYTFGQGTKLEIKGSTSGSGKPGSGEGSTKGQVQLQESGPGLVKPSGTLSLTCAVSGGSISSSNWWSWVRQPPGKGLEWLGEISHSGITNYNPSLKSRVTISVDKSKNHFSLKLNSVTAADTAVYYCTKKWELLAFDFWGQGTMVTVSSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKVMADRIIGMRTQLVSNLKKEGSTHNWQHITDQIGMFCFTGLKPEQVERLIKEFSIYMTKDGRISVAGVTSSNVGYLAHAIHQVTK

SEQ ID NO: 97 - protein, 1205 aa, synthetic construct
MALPVTALLLPLALLLHAARPDIVMTQSPLSQPVTPGEPASISCRSSQSLLHRYGYNSLHWYLQKPGQSPQLLIYLGSNRASGVPDRFSGSGSGTDFTLKVSRVEAEDVGVYYCMQALQTPYTFGQGTKLEIKGSTSGSGKPGSGEGSTKGQVQLQESGPGLVKPSGTLSLTCAVSGGSISSSNWWSWVRQPPGKGLEWLGEISHSGITNYNPSLKSRVTISVDKSKNHFSLKLNSVTAADTAVYYCTKKWELLAFDFWGQGTMVTVSSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKVMADRIIGMRTQLVSNLKKEGSTHNWQHITDQIGMFCFTGLKPEQVERLIKEFSIYMTKDGRISVAGVTSSNVGYLAHAIHQVTKGSGEGRGSLLTCGDVEENPGPMARFALTVVRHGETRFNKEKIIQGQGVDEPLSETGFKQAAAAGIFLNNVKFTHAFSSDLMRTKQTMHGILERSKFCKDMTVKYDSRLRERKYGVVEGKALSELRAMAKAAREECPVFTPPGGETLDQVKMRGIDFFEFLCQLILKEADQKEQFSQGSPSNCLETSLAEIFPLGKNHSSKVNSDSGIPGLAASVLVVSHGAYMRSLFDYFLTDLKCSLPATLSRSELMSVTPNTGMSLFIINFEEGREVKPTVQCICMNLQDHLNGLTETR

(19) United States
(12) Patent Application Publication
(10) Pub. No.: US 2026/0053924 A1
(43) Pub. Date: Feb. 26, 2026

(54) GENETICALLY ENGINEERED IMMUNE CELLS WITH CHIMERIC RECEPTOR POLYPEPTIDES IN COMBINATION WITH MULTIPLE TRANS METABOLISM MOLECULES AND THERAPEUTIC USES THEREOF

(71) Applicant: Sotio Biotech Inc., Boston, MA (US)

(72) Inventors: Samyabrata BHADURI, Boston, MA (US); Emily KUIPER, Boston, MA (US); Amy JENSEN, Boston, MA (US)

(21) Appl. No.: 19/104,547

(22) PCT Filed: Aug. 18, 2023
(86) PCT No.: PCT/US2023/072445

§ 371 Date: Feb. 18, 2025

Related U.S. Application Data
(60) Provisional application No. 63/399,424, filed on Aug. 19, 2022.

(51) Int. Cl. A61K 40/11; A61K 40/15; A61K 40/31; A61K 40/42; A61P 35/00; C07K 14/705; C07K 16/28; C07K 16/30; C12N 5/0783; C12N 9/10; C12N 9/16; C12N 15/86

(52) U.S. Cl. CPC A61K 40/11; A61K 40/15; A61K 40/31; A61K 40/421; A61K 40/4261; A61P 35/00; C07K 14/705; C07K 16/2803; C07K 16/303; C12N 5/0636; C12N 5/0646; C12N 9/1096; C12N 9/16; C12N 15/86; C12Y 206/01007; C12Y 301/03046; C07K 2317/53; C07K 2317/622; C07K 2319/02; C07K 2319/03; C12N 2510/00; C12N 2740/10043; C12N 2740/10052

(57) ABSTRACT

Genetically engineered immune cells, which express at least two metabolism modulating polypeptides and optionally a chimeric receptor polypeptide (e.g., an antibody-coupled T cell receptor (ACTR) polypeptide or a chimeric antigen receptor (CAR) polypeptide) capable of binding to a target antigen of interest. Also disclosed herein are uses of the engineered immune cells for inhibiting cells expressing a target antigen in a subject in need thereof.

### CROSS REFERENCE TO RELATED APPLICATIONS

This application claims the benefit of the filing dates of U.S. Provisional Application No. 63/399,424, filed Aug. 19, 2022, the entire contents of which are incorporated by reference herein.

### SEQUENCE LISTING

The instant application contains a Sequence Listing which has been filed electronically in XML format and is hereby incorporated by reference in its entirety. Said XML copy, created on Aug. 17, 2023, is named 112309-0121-70007WO00_SEQ.xml and is 97,881 bytes in size.

### FIELD OF THE INVENTION

The present invention relates to genetically modified immune cells expressing a chimeric receptor polypeptide (e.g., Chimeric Antigen Receptor, CAR) and a metabolism modulating polypeptide. The present invention further relates to a CAR-NK cell or CAR-T cell and their use in particular for treating cancer.

### BACKGROUND OF DISCLOSURE

Cancer immunotherapy, including cell-based therapy, is used to provoke immune responses attacking tumor cells while sparing normal tissues. It is a promising option for treating various types of cancer because of its potential to evade genetic and cellular mechanisms of drug resistance, and to target tumor cells while sparing normal tissues.

Cell-based therapy may involve cytotoxic T cells having reactivity skewed toward cancer cells (Eshhar et al., Proc Natl Acad Sci USA, 90(2): 720-724 (1993); Geiger et al., The Journal of Immunology, 162(10): 5931-5939 (1999); Brentjens et al., Nat Med, 9(3): 279-286 (2003); Cooper et al., Blood, 101(4): 1637-1644 (2003); Imai et al., Leukemia, 18(4): 676-684 (2004)).

While cell-based immune therapies have shown promising therapeutic effects, they have faced challenges caused by specific characteristics of the tumor microenvironment (TME), which is cellular environment created via the interaction between malignant tumor cells and non-transformed cells.

It is therefore of great importance to develop strategies to improve efficacy of cell-based immune therapies in light of the TME.

### SUMMARY OF DISCLOSURE

The present disclosure is based on the development of strategies to divert or redirect glucose metabolites, increase glucose uptake, modulate glycolysis, the Krebs cycle, intracellular lactate concentration, amino acid uptake and/or its conversion in as immune cells, including those that express a chimeric receptor polypeptide, such as an antibody-coupled T-cells receptor (ACTR) polypeptide or a chimeric antigen receptor (CAR) polypeptide, for use in cell-based immune therapy. Such metabolic modulation may be achieved by expressing or over-expressing at least two of metabolism modulating polypeptides (e.g., those disclosed herein) in combination in immune cells (e.g., αβ or γδ T cells or NK cells), such as those described herein.

The present invention relates to genetically engineered immune cells that are expected to have an enhanced metabolic activity relative to native immune cells of the same type, for example, in a low glucose, low amino acid, low pH, and/or hypoxic environment (e.g., in a TME). As such, the immune cells co-express at least two metabolism modulating polypeptides. The metabolism modulating polypeptides are encoded by an exogenous nucleic acid introduced into the immune cells. Exemplary metabolism modulating polypeptides include TP53-inducible glycolysis and apoptosis regulator (TIGAR), glucose importation factor Glucose Transporter 1 (GLUT1), Glutamic-oxaloacetic transaminase 2 (GOT2), L-Lactate Dehydrogenase A (LDHA), Pyruvate dehydrogenase Kinase 1 (PDK1), Cystathionine gamma-lyase (CTH), Argininosuccinate synthase (ASS1) and Phosphoserine Phosphatase (PSPH). Immune cells co-expressing at least two metabolism modulating polypeptides such as those disclosed herein and a chimeric receptor polypeptide would exhibit superior bioactivities (e.g., under low glucose, low amino acid, low pH, and/or hypoxic conditions) in the presence of a therapeutic antibody, for example, cell proliferation, activation (e.g., increased cytokine production, e.g., IL-2 or IFN-γ production), cytotoxicity, and/or in vivo anti-tumor activity. Such superior bioactivities are especially beneficial to maintain and/or increase the cytotoxic phenotype of the tumor infiltrating immune cells in the TME and/or avoid exhaustion of such tumor infiltrating immune cells in the TME.

In some embodiments, the metabolism modulating polypeptide redirecting glucose metabolites may divert or redirect substrates out of the glycolysis pathway indirectly by decreasing the rate of glucose breakdown in the glycolysis pathway. Examples include TIGAR or GLUT1. In some embodiments, the metabolism modulating polypeptide modulates the Krebs cycle via an enzyme that catalyzes a reaction of the Krebs cycle. One example is GOT2. In some embodiments the metabolism modulating polypeptide is an enzyme involved in lactate synthesis, for example, LDHA. In other embodiments, the metabolism modulating polypeptide is an enzyme that inhibits a pathway that competes for lactate-synthesis substrates, for example, PDK1. In yet other embodiments, the metabolism modulating polypeptides modulate the intracellular concentration of amino acids by increasing amino acid synthesis. Examples include CTH, ASS1 and PSPH.

Provided herein are selected metabolism modulating polypeptides in combination that surprisingly enhanced metabolic readouts in immune cells genetically modified by expressing/overexpressing nucleic acids encoding such metabolism modulating polypeptides. The modified immune cells further express a chimeric receptor polypeptide, which comprises (a) an extracellular target binding domain; (b) a transmembrane domain; and (c) a cytoplasmic signaling domain. Any of the chimeric polypeptides disclosed herein may further comprise at least one co-stimulatory signaling domain. In other embodiments, the chimeric receptor polypeptide may be free of co-stimulatory signaling domains. In one embodiment, the disclosure relates to a genetically engineered immune cell, which (i) express or overly expresses at least two metabolism modulating polypeptides selected from the group consisting of GOT2, GLUT1, LDHA, PDK1, TIGAR, CTH, ASS1 and PSPH; and (ii) expresses a chimeric receptor polypeptide; wherein the chimeric receptor polypeptide comprises (a) an extracellular target binding domain; (b) a transmembrane domain; and (c) at least one cytoplasmic signaling domain. Any of the chimeric polypeptides disclosed herein may further comprise at least one co-stimulatory signaling domain. In other embodiments, the chimeric receptor polypeptide may be free of co-stimulatory signaling domains. In another embodiment, the disclosure relates to a genetically engineered immune cell, wherein the two metabolism modulating polypeptides are selected from the group consisting of: (a) GOT2 and TIGAR; (b) GOT2 and GLUT1; (c) GOT2 and PDK1; (d) TIGAR and GLUT1; (e) PDK1 and CTH; (f) CTH and PSPH; (g) GLUT1 and ASS1; and (h) GLUT1 and PSPH.

In some embodiments, the chimeric receptor polypeptide is an antibody-coupled T cell receptor (ACTR), which comprises an extracellular Fc-binding domain (a). In other embodiments, the chimeric receptor is a chimeric antigen receptor (CAR), which comprises an extracellular antigen binding domain (a). In specific embodiments, the genetically engineered immune cells (e.g., NK, αβ T or γδ T cell) described herein may comprise a nucleic acid or a nucleic acid set, which collectively comprises: (A) a first nucleotide sequence encoding one of the at least two metabolism modulating polypeptides of (i); (B) a second nucleotide sequence encoding the other one of the at least two metabolism modulating polypeptides as disclosed herein; and (C) a third nucleotide sequence encoding the chimeric receptor polypeptide of as also disclosed herein.

In another aspect, the present disclosure provides a pharmaceutical composition, comprising any of the immune cells of the present invention described herein and a pharmaceutically acceptable carrier. Moreover, provided herein is a method for inhibiting and/or killing cells expressing a target antigen (e.g., reducing the number of such cells, blocking cell proliferation, and/or suppressing cell activity) in a subject, the method comprising administering to a subject in need thereof a population of the immune cells described herein. The subject (e.g., a human patient such as a human patient suffering from a cancer) may have been treated or is being treated with an anti-cancer therapy (e.g., an anticancer agent). In some examples, at least some of the cells expressing the target antigen are located in a low-glucose environment, a low-amino acid (e.g., low glutamine) environment, a low-pH environment, and/or a hypoxic environment, for example a tumor microenvironment. In one embodiment, the disclosure relates to a nucleic acid or nucleic acid set, which collectively comprises: (A) a first nucleotide sequence encoding the one of the at least two metabolism modulating polypeptides (i); (B) a second nucleotide sequence encoding the other of the at least two metabolism modulating polypeptides (i); and (C) a third nucleotide sequence encoding the chimeric receptor polypeptide.

Also, within the scope of the present disclosure are uses of the genetically engineered immune cells of the present invention for treating a target disease or disorder such as cancer or an infectious disorder, and uses thereof for manufacturing a medicament for the intended medical treatment.

The details of one or more embodiments of the disclosure are set forth in the description below. Other features or advantages of the present disclosure will be apparent from the detailed description of several embodiments and from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1: Increased glucose uptake by T cells transduced with GLUT1, GOT2 and TIGAR relative to a mock transduced control (null) as a measure of fold change in luminescence.

FIG. 2: Free lactate produced by T cells transduced with GLUT1, GOT2 and TIGAR relative to mock transduced control (null) in the absence (“unstimulated”) or presence of stimulation with PMA and ionomycin (“PMA+Ionomycin”) as a measure of fold change in luminescence.

FIG. 3: Immunoblot showing transgene expression upon retroviral transduction with CAR only or CAR and transgene(s) (GOT2 and/or TIGAR) relative to mock transduced control (null) in NK92 cells. β-ACTIN is shown as protein loading control.

FIGS. 4A-4E: Flow cytometric plots depicting CAR expression upon retroviral transduction with CAR only or CAR and transgene (GOT2 and TIGAR) relative to mock transduced control (null) in NK92 cells. FIG. 4A: Untransduced control. FIG. 4B: CAR only. FIG. 4C: CAR and TIGAR. FIG. 4D: CAR and GOT2. FIG. 4E: CAR and GOT2 and TIGAR.

FIG. 5: Schematic representation of chimeric antigen receptor (CAR) expression constructs targeting human ROR1 and co-expressing metabolism modulating polypeptide(s) separated by a self-cleaving peptide(s). Expression construct 1730 encodes a CAR targeting ROR1 comprising the CD8a signaling domain, the anti-ROR1 scFv, the IgG4 hinge domain, the CD28 transmembrane domain, the 4-1BB co-stimulatory domain and the CD35 cytoplasmic domain; expression constructs 1767 encodes the same CAR as in clone 1730. The coding sequence of the CAR is separated from the transgene TIGAR by a coding sequence of P2A; expression construct 1768 encodes the same CAR as in clone 1730. The coding sequence of the CAR is separated from the transgene GOT2 by a coding sequence of P2A; expression construct 1798 comprises the same CAR as in clone 1730. The coding sequence of the CAR is separated from the transgene GOT2 by a coding sequence of P2A and from the transgene TIGAR by a coding sequence of T2A.

FIGS. 6A-6D: IFN-γ production by CAR-T cells expressing CAR only (1730), CAR and GOT2 (1768) or CAR, GOT2 and TIGAR (1798). Percent CAR expression was normalized for transduced cells by spiking with untransduced T cells (UTD). CAR T cells were then co-cultured with target tumor cell lines (A-D) in (E:T::1:1) and incubated for 24 h at 37° C. Supernatants were collected and IFN-γ was measured using ELISA. FIG. 6A: K562 cells, derived from CML, negative for ROR1. FIG. 6B: A549, derived from NSCLC, endogenously expressing ROR1. FIG. 6C: CAKI-1, derived from RCC, endogenously expressing ROR1. FIG. 6D: K562-hROR1, engineered to express human ROR1. Data are generated from 4 PBMC donors and are represented as mean+/−SEM.

FIGS. 7A-7D: In vitro cytotoxicity of untransduced T cells, CAR-T cells expressing an anti-ROR1 CAR only (1730), following repeated stimulation. CAR and GOT2 (1768) or CAR, GOT2 and TIGAR (1798) against A549 tumor cells following repeated stimulation.

Cytotoxicity was assessed by enumerating the tumor cell count (red signal). Data were generated from 4 PBMC donors and are shown as cell counts as a function of time in h (FIGS. 7A to 7D).

FIGS. 8A and 8B. In vivo anti-tumor efficacy of CAR-T cells expressing only an anti-ROR1 CAR (1730), the anti-ROR1 CAR and GOT2 (1768), the anti-ROR1 CAR and TIGAR (1767), or the anti-ROR1 CAR, GOT2, and TIGAR (1798) in a CAKI-1 xenograft mouse model. Control animals were left untreated, UTD for treatment with untransduced T cells. FIG. 8A: CAR-T cells derived from Donor 1. FIG. 8B: CAR-T cells derived from Donor 2.

FIGS. 9A and 9B. In vivo tumor infiltrating human CD3⁺ T cells (A) and exhausted CD8⁺ CAR⁺ T cells (B) expressing anti-ROR1 CAR alone (1730), the anti-ROR1 CAR and GOT2 (1768), or the anti-ROR1 CAR, GOT2, and TIGAR (1798) in the CAKI-1 xenograft mouse model. Data represent the average of two individual animals from the same study.

### DETAILED DESCRIPTION OF DISCLOSURE

Immune cell therapy involving genetically engineered T cells has shown promising effects in cancer therapy.

One approach is to express a chimeric receptor having an antigen-binding domain (CAR) fused to one or more T cell activation signaling domains in a (αβ) T cell (a CAR T cell or CAR T). Binding of a cancer antigen via the antigen-binding domain of the CAR expressed on the surface of a T cell results in T cell activation and triggers cytotoxicity. Recent results of clinical trials with infusions of chimeric receptor-expressing autologous T lymphocytes provided compelling evidence of their clinical potential. (Brentjens, Latouche et al., Nat Med, 9(3): 279-286 (2003); Pule et al., Nat Med, 14(11): 1264-1270 (2008); Brentjens et al., Blood, 118(18): 4817-4828 (2011); Porter et al., New England Journal of Medicine, 365(8): 725-733 (2011); Kochenderfer et al., Blood, 119(12): 2709-2720 (2012); Till et al., Blood, 119(17): 3940-3950 (2012); Brentjens et al., Sci Transl Med, 5(177): 177ra138 (2013)). Initially, the field was focusing on ab T cells. More recently, cell-based therapy has expanded to include natural killer (NK) cells due to their unique advantages such as low risk of on-target/off-tumor toxicity in normal tissues, cytokine release syndrome and neurotoxicity. They also exhibit natural cytotoxicity against tumor cells. Finally, due to reduced graft versus host disease (GVHD), an allogeneic/off-the-shelf cell therapy product may be prepared (Schmidt et al., Front Immunol, 11:611163 (2020); Wang et al., Cancer Lett, 472:175-180 (2020); Xie et al., EBioMedicine, 59:102975 (2020); Gong et al., J Hematol Oncol, 14(1): 73 (2021); Wrona et al., Int J Mol Sci, 22(11): (2021)). Another subtype of T cells, i.e., γδ T cells, have immense potential in cell therapy sharing similar advantages of that of NK cells additionally displaying immune regulatory functions (Sievers et al., Int J Mol Sci, 21(10): (2020); Park and Lee, Exp Mol Med, 53(3): 318-327 (2021)). In addition, persistent activation of these αβ T cells due to antigen dependent or independent CAR activation can lead to exhaustion and reduced bioactivity which is again an advantage of the innate cells such as NK and γδ T cells. Finally, both NK and γδ T cells expressing a chimeric receptor polypeptide may have increased effector functions such as increased inflammatory cytokine production, antigen acquisition and presentation or ability to activate adaptive immune responses.

Another approach is to express an antibody-coupled T cell Receptor (ACTR) protein in a hematopoietic cell (e.g., a hematopoietic stem cell, an immune cell, such as an NK cell or a T cell), the ACTR protein containing an extracellular Fc-binding domain. When the ACTR-expressing hematopoietic cells (e.g., ACTR-expressing T cells, also called “ACTR T cells”) are administered to a subject together with an anti-cancer antibody, they may enhance toxicity against cancer cells targeted by the antibody via their binding to the Fc domain of the antibody (Kudo et al., Cancer Res, 74(1): 93-103 (2014)).

Tumor microenvironments (TME) have specific characteristics, such as low glucose, low amino acid, low pH, and/or hypoxic conditions, some of which may constrain the activity of effector immune cells (e.g., effector T or NK cells). The present disclosure is based, at least in part, on the development of strategies for enhancing effector immune cell activities in the TME. In particular, the present disclosure features methods for enhancing the metabolic activity of the effector immune cells (e.g., diverting or re-directing one or more glucose metabolites out of the glycolysis pathway) in the effector immune cells, thereby enhancing their growth and bioactivity.

The present disclosure provides genetically engineered immune cells (e.g., NK, αβ T or γδ T cells) that possess altered glucose metabolism and/or uptake, lactate production and enhanced amino acid synthesis as compared with a native immune cell of the same type. Accordingly, provided herein are modified (e.g., genetically engineered) immune cells (e.g., αβ or γδ T cells, or NK cells) that have, e.g., altered intracellular regulation of glucose concentrations, capacity for an increased rate of glycolysis or intracellular lactate concentrations relative to the wild-type immune cells of the same type. In some instances, the modified immune cells may express or overly express the metabolism modulating polypeptides for example, a polypeptide that diverts or redirects glucose metabolites out of the glycolysis pathway. Alternatively, the modified immune cell may be engineered to transfect at least one exogenous nucleic acid encoding at least two metabolism modulating polypeptides for producing additional amount of the polypeptide in the modified immune cell.

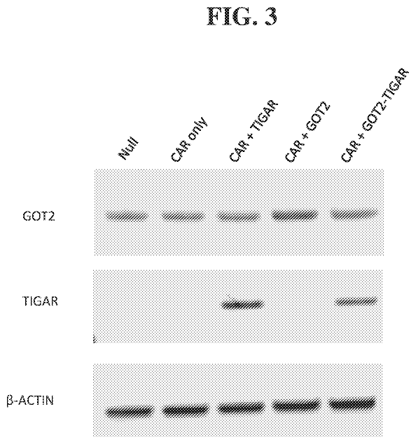

Such genetically engineered immune cells express or overly express at least two metabolism modulating polypeptides combined specifically to enhance the metabolism in the immune cells and express a chimeric receptor polypeptide comprising an extracellular target binding domain, a transmembrane domain and a cytoplasmic signaling domain, e.g., an antibody-coupled T cell receptor (ACTR) polypeptide or, preferably, a chimeric antigen receptor (CAR) polypeptide. A modified immune cell expressing at least two polypeptides refers to a genetically engineered immune cell into which one or more exogenous nucleic acids encoding at least two metabolism modulating polypeptides are introduced such that the encoded metabolism modulating polypeptides are expressed in the resultant modified immune cell, while the unmodified parent cell does not express such metabolism modulating polypeptides. For example, TIGAR was found to be not detectable by immunoblotting in mock transduced NK92 cells (see FIG. 3). A modified immune cell overly expressing at least two of the polypeptides that modulate metabolism refers to a genetically engineered immune cell, which is engineered to enhance the expression level of the metabolism modulating polypeptides as relative to the unmodified parent cell. For example, GOT2 was found have a basic expression in mock transduced NK92 cells, whereas retroviral transduction with CAR and GOT2 lead to a detectable increased GOT2 expression using immunoblotting (see FIG. 3). In some instances, the modified immune cell may be engineered to express one of the metabolism modulating polypeptide and overly express another metabolism modulating polypeptide, expresses two metabolism modulating polypeptides, overly express two metabolism modulating polypeptides, or enhance expression of the endogenous gene encoding at least two metabolism modulating polypeptides. In a preferred embodiment, such metabolism modulating polypeptide(s) is/are overly expressed compared to a native immune cell of the same type, e.g., by polypeptides encoded by transgene(s) introduced into the immune cells (e.g., exogenous to the immune cells). Expression or overexpression can be determined in analogy as shown for TIGAR and GOT2 respectively as shown in Example 17.

Also, provided herein are uses of the genetically engineered immune cells, for improving immune cell proliferation, and/or an inhibiting or decreasing target cells (e.g., target cancer cells) in a subject (e.g., a human cancer patient), e.g., via CAR-T mediated or ACTR mediated cell killing.

As such, the genetically engineered immune cells may proliferate better, produce more, preferably cytotoxic, cytokines, exhibit greater anti-tumor cytotoxicity, and/or exhibit greater survival of the respective genetically engineered immune cells in a low-glucose, low amino acid, low pH, and/or hypoxic environment (e.g., a TME) relative to immune cells that do not express or do not over-express the at least two metabolism modulating polypeptides selected from GOT2, GLUT1, LDHA, PDK1, TIGAR, CTH, ASS1 and PSPH, as further described below, leading to enhanced cytokine production, survival rate, cytotoxicity, and/or anti-tumor activity.

### I. Metabolism Modulating Polypeptides

As used herein, the term “metabolism modulating polypeptide” refer to polypeptides that regulate a metabolism pathway, for example, redirecting glucose metabolites out of the glycolysis pathway, increasing glucose uptake, modulating Krebs cycle, modulating intracellular lactate concentration, increasing amino acid uptake and/or its conversion. Exemplary metabolism modulating polypeptides for use in making the genetically engineered immune cells disclosed herein may include GOT2, GLUT1, LDHA, PDK1, TIGAR, CTH, ASS1 and PSPH. Any combination of two or more metabolism modulating polypeptides, e.g., selected from the list provided herein, may be used in the present disclosure.

In some instances, one of the at least two metabolism modulating polypeptides reduces the function of an enzyme in the glycolysis pathway. Such a metabolism modulating polypeptide is TIGAR (disclosed in WO2023/049933A1, the relevant disclosures of which are incorporated by reference for the subject matter and purpose referenced herein). TIGAR functions to block glycolysis and re-direct glucose metabolites into the pentose phosphate shunt pathway. TIGAR is in direct opposition with PFKFB3 with respect to their shared regulation of fructose-2,6-bisphosphate, a molecule that increases the activity of the glycolytic pathway enzyme PFK. TIGAR degrades fructose-2,6-bisphosphate which effectively slows down the enzymatic rate of PFK. This allows for more glucose metabolites to be re-directed into nucleotide synthesis and glycosylation pathways, e.g., the pentose phosphate shunt pathway. Elevated TIGAR expression or activity levels increase the redirection of glucose metabolites away from the glycolysis pathway. The amino acid sequence of an exemplary human TIGAR enzyme is provided below (SEQ ID NO: 75). The amino acid sequence of an exemplary human TIGAR is provided in Table 1 below. In specific embodiments, TIGAR may be paired with GOT2 or GLUT1 for making the genetically engineered immune cells disclosed herein. In a specific embodiment, TIGAR is human TIGAR (e.g., SEQ ID NO: 75). The term “TIGAR” encompasses functional equivalents of TIGAR, whereas a functional equivalent of TIGAR is a polypeptide having at least 85%, preferably at least 90%, more preferably at least 95% sequence identity with human TIGAR (e.g., SEQ ID NO: 75).

In some instances, one of the at least two metabolism modulating polypeptides modulates the Krebs cycle and/or links various metabolic pathways such as the metabolic pathways for processing glucose, amino acids and/or fatty acids. In one example, the metabolism modulating polypeptide is GOT2 (disclosed in WO2020/037066, the relevant disclosures of which are incorporated by reference for the subject matter and purpose referenced herein). The GOT2 polypeptide modulates the Krebs cycle as a metabolic substrate located within the inner mitochondria. The amino acid sequence of an exemplary human GOT2 enzyme is provided below (SEQ ID NO: 77). The amino acid sequence of an exemplary human GOT2 is provided in Table 1 below. In specific embodiments, GOT2 may be paired with TIGAR or GLUT1 for use in making the genetically modified immune cells disclosed herein. In a specific embodiment, GOT2 is human GOT2 (e.g., SEQ ID NO: 77). The term “GOT2” encompasses functional equivalents of GOT2 (, whereas a functional equivalent of GOT2 is a polypeptide having at least 85%, preferably at least 90%, more preferably at least 95% sequence identity with human GOT2 (e.g., SEQ ID NO: 77).

In some instances, one of the at least two metabolism modulating polypeptides mediates glucose uptake (i.e., increases glucose import) across the plasma membrane of cells, which is also known as glucose importation polypeptides. One such glucose importation polypeptide that increases glucose uptake is the class I Glucose Transporter 1 (GLUT1; disclosed in WO2020/010110, the relevant disclosures of which are incorporated by reference for the subject matter and purpose referenced herein). The amino acid sequence of an exemplary human GLUT1 enzyme is provided below (SEQ ID NO: 76). The amino acid sequence of an exemplary human GLUT1 is provided in Table 1 below. In a specific embodiment, GLUT1 may be paired with GOT2, TIGAR ASS1, or PSPH for used in making any of the genetically modified immune cells. In a specific embodiment, GLUT1 is, human GLUT1 (e.g., SEQ ID NO: 76). The term “GLUT1” encompasses functional equivalents of GLUT1, whereas a functional equivalent of GLUT1 is a polypeptide having at least 85%, preferably at least 90%, more preferably at least 95% sequence identity with human GLUT1 (e.g., SEQ ID NO: 76).

In some instances, one of the at least two metabolism modulating polypeptides is a lactate modulating factor that can be either involved in lactate synthesis. In one example, the metabolism modulating polypeptide LDHA (disclosed in WO2020/051493, the relevant disclosures of which are incorporated by reference for the subject matter and purpose referenced herein). LDHA is a dehydrogenase enzyme that catalyzes the interconversion of pyruvate, a key molecule in the Krebs cycle, and lactate. The over-expression of LDHA may facilitate the conversion of lactate into pyruvate as a cell's store of pyruvate is diminished at times of high metabolic activity. This leads to an increase in the intracellular concentration of pyruvate and a decrease in the intracellular concentration of lactate and has the effect of providing flux into the Krebs cycle and increasing the transport of lactate. Accordingly, elevated expression or activity of LDHA increases the transport of lactate, leading to an ultimate elevated intracellular lactate concentration. The amino acid sequence of an exemplary human LDHA is provided in Table 1 below. In specific embodiments, the LDHA such as human LDHA may be paired with GOT2 for use in making any of the genetically engineered immune cells disclosed herein. The term “LDHA” encompasses functional equivalents of LDHA, whereas a functional equivalent of LDHA is a polypeptide having at least 85%, preferably at least 90%, more preferably at least 95% sequence identity with human LDHA (e.g., SEQ ID NO: 78).

Alternatively, one of the at least two metabolism modulating polypeptides is an enzyme that inhibits a pathway competing for substrates used in lactate synthesis. Such a metabolism modulating polypeptide may be PDK1 (disclosed in WO2020/051493, the relevant disclosures of which are incorporated by reference for the subject matter and purpose referenced herein). PDK1 is a kinase which acts to inhibit pyruvate dehydrogenase (such as PDHA1), a component of the pyruvate dehydrogenase complex, via phosphorylation. The pyruvate dehydrogenase complex converts pyruvate into acetyl-CoA through decarboxylation. Increased PDK1 expression or activity—and subsequent inhibition of pyruvate dehydrogenase—increases the amount of pyruvate available for LDHA-mediated conversion to lactate.

The amino acid sequence of an exemplary human PDK1 is provided in Table 1 below (SEQ ID NO: 79). In specific embodiments, a PDK1 polypeptide such as human PDK1 may be paired with GOT2, PDK1 or CTH for use in any of the genetically modified immune cells. The term “PDK1” encompasses functional equivalents of PDK1, whereas a functional equivalent of PDK1 is a polypeptide having at least 85%, preferably at least 90%, more preferably at least 95% sequence identity with human PDK1 (e.g., SEQ ID NO: 79).

In some instances, one of the at least two metabolism modulating polypeptides is capable of modulating the intracellular concentration of amino acids. Examples include ASS1, PSHP or CTH. These polypeptides modulate the intracellular concentration of amino acids and increase amino acid synthesis (e.g., an enzyme that stimulates amino acid synthesis or the conversion of an amino acid into another molecule). ASS1 catalyzes the penultimate step of the arginine biosynthetic pathway, PSPH catalyzes magnesium-dependent hydrolysis of L-phosphoserine and is also involved in an exchange reaction between L-serine and L-phosphoserine, and CTH catalyzes the last step of the trans-sulfuration pathway, interconverting cystathionine with cysteine. The amino acid sequence of an exemplary human ASS1, PSPH and CTH are provided in Table 1 below (SEQ ID NO: 80-SEQ ID NO: 82). In specific embodiments, a pair of CTH and PSPH, ASS1 and GLUT1, PSPH and GLUT1, or CTH and PDK1 may be used in any of the genetically modified immune cells. In some examples, human ASS1 (e.g., SEQ ID NO: 80), human PSPH (e.g., SEQ ID NO: 81) and/or human CTH (e.g., SEQ ID NO: 82) may be used. The term “ASS1” encompasses functional equivalents of ASS1, whereas a functional equivalent of ASS1 is a polypeptide having at least 85%, preferably at least 90%, more preferably at least 95% sequence identity with human ASS1 (e.g., SEQ ID NO: 80). The term “PSPH” encompasses functional equivalents of PSPH, whereas a functional equivalent of PSPH is a polypeptide having at least 85%, preferably at least 90%, more preferably at least 95% sequence identity with human PSPH (e.g., SEQ ID NO: 81). The term “CTH” encompasses functional equivalents of CTH (e.g., SEQ ID NO: 82), whereas a functional equivalent of CTH is a polypeptide having at least 85%, preferably at least 90%, more preferably at least 95% sequence identity with human CTH (e.g., SEQ ID NO: 82).

Specific non-limiting examples of metabolism modulating polypeptides for use in the present disclosure include: (a) GOT2 and TIGAR; (b) GOT2 and GLUT1; (c) GOT2 and PDK1; (d) TIGAR and GLUT1; (e) PDK1 and CTH; (f) CTH and PSPH; (g) GLUT1 and ASS1; and (h) GLUT1 and PSPH. Especially preferred is the GOT2 and TIGAR combination. CAR Ts co-expressing in the meaning of this invention (e.g., over-expressing GOT2 and expressing TIGAR) with an anti-ROR1 CAR showed superior anti-tumor efficacy in vivo and beneficial CAR T phenotypes for tumor infiltrating CAR⁺ lymphocytes (see Example 17).

The metabolism modulating polypeptides may be naturally-occurring polypeptides from a suitable species, for example, a mammalian glucose importation polypeptide such as those derived from human or a non-human primate. Such naturally-occurring polypeptides are known in the art and can be obtained, for example, using any of the above-noted amino acid sequences as a query to search a publicly available gene database, for example GenBank. The metabolism modulating polypeptides for use in the instant disclosure may share a sequence identity of at least 85% (e.g., 90%, 95%, 97%, 98%, 99%, or above) as any of the exemplary proteins noted above.

The “percent identity” of two amino acid sequences is determined using the algorithm of (Karlin and Altschul, Proc Natl Acad Sci USA, 87(6): 2264-2268 (1990)), modified as in (Karlin and Altschul, Proc Natl Acad Sci USA, 90(12): 5873-5877 (1993)). Such an algorithm is incorporated into the NBLAST and XBLAST programs (version 2.0) of (Altschul et al., J Mol Biol, 215(3): 403-410 (1990)). BLAST protein searches can be performed with the XBLAST program, score=50, wordlength=3 to obtain amino acid sequences homologous to the protein molecules of the invention. Where gaps exist between two sequences, Gapped BLAST can be utilized as described in (Altschul et al., Nucleic Acids Res, 25(17): 3389-3402 (1997)). When utilizing BLAST and Gapped BLAST programs, the default parameters of the respective programs (e.g., XBLAST and NBLAST) can be used.

Alternatively, the metabolism modulating polypeptides may be a functional variant of a native counterpart. Such a functional variant may contain one or more mutations outside the functional domain(s) of the native counterpart. Functional domains of a native metabolism modulating polypeptides may be known in the art or can be predicted based on its amino acid sequence. Mutations outside the functional domain(s) would not be expected to substantially affect the biological activity of the protein. In some instances, the functional variant may exhibit an increased activity (for example, glucose uptake as relative to the native counterpart). Alternatively, the functional variant may exhibit a decreased activity in glucose uptake as relative to the native counterpart. Additionally, the functional variant may have increased trafficking to the cell surface. Alternatively, the functional variant may have decreased trafficking to the cell surface.

Alternatively or in addition, the functional variant may contain a conservative mutation(s)/substitution(s) at one or more positions in the native counterpart (e.g., up to 20 positions, up to 15 positions, up to 10 positions, up to 5, 4, 3, 2, 1 position(s)). As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Conservative Mutations that result still in functional variants include besides conservative substitutions small (e.g., 1 to 3 amino acids) insertions, deletions or inversions that do not result in altered relative charge or does not substantially change the size of the protein.

Table 1 provides amino acid sequences of exemplary polypeptides that redirect glucose metabolites out of glycolysis pathway.

TABLE 1
Exemplary Polypeptides for Modulating Metabolism

| | | SEQ |
|---|---|---|
| Polypeptides | Sequences | ID NO |
| TIGAR | MARFALTVVRHGETRFNKEKIIQGQGVDEPLSETGFKQAAAAGIFLNNV | 75 |
| | KFTHAFSSDLMRTKQTMHGILERSKFCKDMTVKYDSRLRERKYGVVEGK | |
| | ALSELRAMAKAAREECPVFTPPGGETLDQVKMRGIDFFEFLCQLILKEA | |
| | DQKEQFSQGSPSNCLETSLAEIFPLGKNHSSKVNSDSGIPGLAASVLVV | |
| | SHGAYMRSLFDYFLIDLKCSLPATLSRSELMSVTPNTGMSLFIINFEEG | |
| | REVKPTVQCICMNLQDHLNGLTETR | |
| GLUT1 | MEPSSKKLTGRLMLAVGGAVLGSLQFGYNTGVINAPQKVIEEFYNQTWV | 76 |
| | HRYGESILPTTLTTLWSLSVAIFSVGGMIGSFSVGLFVNRFGRRNSMLM | |
| | MNLLAFVSAVLMGFSKLGKSFEMLILGRFIIGVYCGLTTGFVPMYVGEV | |
| | SPTALRGALGTLHQLGIVVGILIAQVFGLDSIMGNKDLWPLLLSIIFIP | |
| | ALLQCIVLPFCPESPRFLLINRNEENRAKSVLKKLRGTADVTHDLQEMK | |
| | EESRQMMREKKVTILELFRSPAYRQPILIAVVLQLSQQLSGINAVFYYS | |
| | TSIFEKAGVQQPVYATIGSGIVNTAFTVVSLFVVERAGRRTLHLIGLAG | |
| | MAGCAILMTIALALLEQLPWMSYLSIVAIFGFVAFFEVGPGPIPWFIVA | |
| | ELFSQGPRPAAIAVAGFSNWTSNFIVGMCFQYVEQLCGPYVFIIFTVLL | |
| | VLFFIFTYFKVPETKGRTFDEIASGFRQGGASQSDKTPEELFHPLGADS | |
| | QV | |
| GOT2 | MALLHSGRVLPGIAAAFHPGLAAAASARASSWWTHVEMGPPDPILGVTE | 77 |
| | AFKRDTNSKKMNLGVGAYRDDNGKPYVLPSVRKAEAQIAAKNLDKEYLP | |
| | IGGLAEFCKASAELALGENSEVLKSGRFVTVQTISGTGALRIGASFLQR | |
| | FFKFSRDVFLPKPTWGNHTPIFRDAGMQLQGYRYYDPKTCGFDETGAVE | |
| | DISKIPEQSVLLLHACAHNPTGVDPRPEQWKEIATVVKKRNLFAFFDMA | |
| | YQGFASGDGDKDAWAVRHFIEQGINVCLCQSYAKNMGLYGERVGAFTMV | |
| | CKDADEAKRVESQLKILIRPMYSNPPLNGARIAAAILNTPDLRKQWLQE | |
| | VKVMADRIIGMRTQLVSNLKKEGSTHNWQHITDQIGMFCFTGLKPEQVE | |
| | RLIKEFSIYMTKDGRISVAGVTSSNVGYLAHAIHQVTK | |
| LDHA | MATLKDQLIYNLLKEEQTPQNKITVVGVGAVGMACAISILMKDLADELA | 78 |
| | LVDVIEDKLKGEMMDLQHGSLFLRTPKIVSGKDYNVTANSKLVIITAGA | |
| | RQQEGESRLNLVQRNVNIFKFIIPNVVKYSPNCKLLIVSNPVDILTYVA | |
| | WKISGFPKNRVIGSGCNLDSARFRYLMGERLGVHPLSCHGWVLGEHGDS | |
| | SVPVWSGMNVAGVSLKTLHPDLGTDKDKEQWKEVHKQVVESAYEVIKLK | |
| | GYTSWAIGLSVADLAESIMKNLRRVHPVSTMIKGLYGIKDDVFLSVPCI | |
| | LGQNGISDLVKVTLTSEEEARLKKSADTLWGIQKELQF | |
| PDK1 | MRLARLLRGAALAGPGPGLRAAGFSRSFSSDSGSSPASERGVPGQVDFY | 79 |
| | ARFSPSPLSMKQFLDFGSVNACEKTSFMFLRQELPVRLANIMKEISLLP | |
| | DNLLRTPSVQLVQSWYIQSLQELLDFKDKSAEDAKAIYERPRRTWLQVS | |
| | SLCCMACKMIFTDTVIRIRNRHNDVIPTMAQGVIEYKESFGVDPVTSQN | |
| | VQYFLDRFYMSRISIRMLLNQHSLLEGGKGKGSPSHRKHIGSINPNCNV | |
| | LEVIKDGYENARRLCDLYYINSPELELEELNAKSPGQPIQVVYVPSHLY | |
| | HMVFELFKNAMRATMEHHANRGVYPPIQVHVTLGNEDLTVKMSDRGGGV | |
| | PLRKIDRLFNYMYSTAPRPRVETSRAVPLAGFGYGLPISRLYAQYFQGD | |
| | LKLYSLEGYGTDAVIYIKALSTDSIERLPVYNKAAWKHYNTNHEADDWC | |
| | VPSREPKDMTTERSA | |
| ASS1 | MSSKGSVVLAYSGGLDTSCILVWLKEQGYDVIAYLANIGQKEDFEEARK | 80 |
| | KALKLGAKKVFIEDVSREFVEEFIWPAIQSSALYEDRYLLGTSLARPCI | |
| | ARKQVEIAQREGAKYVSHGATGKGNDQVRFELSCYSLAPQIKVIAPWRM | |
| | PEFYNRFKGRNDLMEYAKQHGIPIPVTPKNPWSMDENLMHISYEAGILE | |
| | NPKNQAPPGLYTKTQDPAKAPNTPDILEIEFKKGVPVKVTNVKDGTTHQ | |
| | TSLELFMYLNEVAGKHGVGRIDIVENRFIGMKSRGIYETPAGTILYHAH | |
| | LDIEAFTMDREVRKIKQGLGLKFAELVYTGFWHSPECEFVRHCIAKSQE | |
| | RVEGKVQVSVLKGQVYILGRESPLSLYNEELVSMNVQGDYEPTDATGFI | |
| | NINSLRLKEYHRLQSKVTAK | |
| PSPH | MVSHSELRKLFYSADAVCFDVDSTVIREEGIDELAKICGVEDAVSEMTR | 81 |
| | RAMGGAVPFKAALTERLALIQPSREQVQRLIAEQPPHLIPGIRELVSRL | |
| | QERNVQVFLISGGERSIVEHVASKLNIPATNVFANRLKFYENGEYAGED | |
| | ETQPTAESGGKGKVIKLLKEKFHFKKIIMIGDGATDMEACPPADAFIGE | |
| | GGNVIRQQVKDNAKWYITDFVELLGELEE | |
| CTH | MQEKDASSQGFLPHFQHFATQAIHVGQDPEQWTSRAVVPPISLSTTFKQ | 82 |
| | GAPGQHSGFEYSRSGNPTRNCLEKAVAALDGAKYCLAFASGLAATVTIT | |
| | HLLKAGDQIICMDDVYGGTNRYFRQVASEFGLKISFVDCSKIKLLEAAI | |
| | TPETKLVWIETPTNPTQKVIDIEGCAHIVHKHGDIILVVDNTEMSPYFQ | |
| | RPLALGADISMYSATKYMNGHSDVVMGLVSVNCESLHNRLRFLQNSLGA | |
| | VPSPIDCYLCNRGLKTLHVRMEKHFKNGMAVAQFLESNPWVEKVIYPGL | |
| | PSHPQHELVKRQCTGCTGMVTFYIKGTLQHAEIFLKNLKLFTLAESLGG | |
| | FESLAELPAIMTHASVLKNDRDVLGISDTLIRLSVGLEDEEDLLEDLDQ | |
| | ALKAAHPPSGSHS | |

### II. Chimeric Receptor Polypeptides

As used herein, a chimeric receptor polypeptide refers to a non-naturally occurring molecule that can be expressed on the surface of an immune cell and comprises an extracellular target binding domain, a transmembrane domain and at least one cytoplasmic signaling domain. The extracellular target binding domain targets an antigen of interest (e.g., an antigen associated with a disease such as cancer or an antigen associated with a pathogen; see disclosures herein). It may bind to an antigen of interest directly (e.g., an extracellular antigen binding domain in a CAR polypeptide as disclosed herein), or may bind to the antigen of interest via an intermediate, for example, an Fc-containing agent such as an antibody. The transmembrane domain ankers the chimeric receptor polypeptide within the cellular membrane of the immune cell. The chimeric receptor polypeptides are configured such that, when expressed in an immune cell, the extracellular target binding domain is located extracellularly for binding to a target antigen, directly or indirectly, whereas the cytoplasmic signaling domain is located intracellularly to allow for signaling into the cell upon binding of the target binding domain to the target. A chimeric receptor polypeptide may further comprise a hinge domain, one or more co-stimulatory domains, or a combination thereof.

In some embodiments, the chimeric receptor polypeptide comprises one or more of the following features: (i) the chimeric receptor polypeptide further comprises a signal peptide at its N-terminus; (ii) the chimeric receptor polypeptide further comprises a hinge domain, which is located at the C-terminus of (a) and the N-terminus of (b); (iii) the chimeric receptor polypeptide is free of a hinge domain; (iv) the chimeric receptor polypeptide further comprises at least one co-stimulatory signaling domain; (v) the chimeric receptor polypeptide is free of a co-stimulatory signaling domain; and (vi) the cytoplasmic signaling domain comprises an immunoreceptor tyrosine-based activation motif (ITAM).

In some embodiments, a chimeric receptor polypeptide as described herein may comprise, from N-terminus to C-terminus, the extracellular target binding domain, the transmembrane domain, and the cytoplasmic signaling domain. In some embodiments, a chimeric receptor polypeptide as described herein comprises, from N-terminus to C-terminus, the extracellular target binding domain, the transmembrane domain, at least one costimulatory signaling domain, and the cytoplasmic signaling domain. In other embodiments, a chimeric receptor polypeptide as described herein comprises, from N-terminus to C-terminus, the extracellular target binding domain, the transmembrane domain, the cytoplasmic signaling domains, and at least one co-stimulatory signaling domain.

In some embodiments, the chimeric receptor polypeptide can be an antibody-coupled T cell receptor (ACTR) polypeptide. As used herein, an ACTR polypeptide (a.k.a., an ACTR construct) refers to a non-naturally occurring molecule that can be expressed on the surface of an immune cell and comprises an extracellular domain with binding affinity and specificity for the Fc portion of an immunoglobulin (“Fc binder” or “Fc binding domain”), a transmembrane domain, and a cytoplasmic signaling domain. In some embodiments, the ACTR polypeptides described herein may further include at least one co-stimulatory signaling domain.

In other embodiments, the chimeric receptor polypeptide disclosed herein may be a chimeric antigen receptor (CAR) polypeptide. As used herein, a CAR polypeptide (a.k.a., a CAR construct) refers to a non-naturally occurring molecule that can be expressed on the surface of an immune cell and comprises an extracellular antigen binding domain, a transmembrane domain, and a cytoplasmic signaling domain. The CAR polypeptides described herein may further include at least one co-stimulatory signaling domain.

As used herein, the phrase “a protein X transmembrane domain” (e.g., a CD8 transmembrane domain) refers to any portion of a given protein, i.e., transmembrane-spanning protein X, that is thermodynamically stable in a membrane.

As used herein, the phrase “a protein X cytoplasmic signaling domain,” for example, a CD3ζ cytoplasmic signaling domain, refers to any portion of a protein (protein X) that interacts with the interior of a cell or organelle and is capable of relaying a primary signal as known in the art, which leads to immune cell proliferation and/or activation. The cytoplasmic signaling domain as described herein differs from a co-stimulatory signaling domain, which relays a secondary signal for fully activating immune cells.

As used herein, the phrase “a protein X co-stimulatory signaling domain,” e.g., a CD28 co-stimulatory signaling domain, refers to the portion of a given co-stimulatory protein (protein X, such as CD28, 4-1BB, OX40, CD27, or ICOS) that can transduce co-stimulatory signals (secondary signals) into immune cells (such as T cells), leading to fully activation of the immune cells.

### A. Extracellular Target Binding Domain

The chimeric receptor polypeptides disclosed herein comprise an extracellular domain that targets an antigen of interest (e.g., those described herein) via either direct binding or indirectly binding (through an intermediate such as an antibody). The chimeric receptor polypeptides may be ACTR polypeptides that comprise a Fc binding domain. Alternatively, the chimeric receptor polypeptides may be CAR polypeptides that comprise an extracellular antigen binding domain.

### (i) Fc Binding Domains

The ACTR polypeptides described herein comprise an extracellular target binding domain that is an Fc binding domain, i.e., capable of binding to the Fc portion of an immunoglobulin (e.g., IgG, IgA, IgM, or IgE) of a suitable mammal (e.g., human, mouse, rat, goat, sheep, or monkey). Suitable Fc binding domains may be derived from naturally occurring proteins such as mammalian Fc receptors or certain bacterial proteins (e.g., protein A, protein G). Additionally, Fc binding domains may be synthetic polypeptides engineered specifically to bind the Fc portion of any of the antibodies described herein with high affinity and specificity. For example, such an Fc binding domain can be an antibody or an antigen-binding fragment thereof that specifically binds the Fc portion of an immunoglobulin. Examples include, but are not limited to, a single-chain variable fragment (scFv), a domain antibody, or single domain antibodies (e.g., nanobodies). Alternatively, an Fc binding domain can be a synthetic peptide that specifically binds the Fc portion, such as a Kunitz domain, a small modular immunopharmaceutical (SMIP), an adnectin, an avimer, an affibody, a DARPin, or an anticalin, which may be identified by screening a peptide combinatory library for binding activities to Fc.

In some embodiments, the Fc binding domain is an extracellular ligand-binding domain of a mammalian Fc receptor. As used herein, an “Fc receptor” is a cell surface bound receptor that is expressed on the surface of many immune cells (including B cells, T cells and NK cells) and exhibits binding specificity to the Fc domain of an antibody. Fc receptors are typically comprised of at least two immunoglobulin (Ig)-like domains with binding specificity to an Fc (fragment crystallizable) portion of an antibody. In some instances, binding of an Fc receptor to an Fc portion of the antibody may trigger antibody dependent cell-mediated cytotoxicity (ADCC) effects. The Fc receptor used for constructing an ACTR polypeptide as described herein may be a naturally occurring polymorphism variant (e.g., the CD16 V158 variant), which may have increased or decreased affinity to Fc as compared to a wild-type counterpart. Alternatively, the Fc receptor may be a functional variant of a wildtype counterpart, which carry one or more mutations (e.g., up to 10 amino acid residue substitutions including 1, 2, 3, 4, 5, 6, 7, 8, 9, or 10 mutations) that alter the binding affinity to the Fc portion of an Ig molecule. In some instances, the mutation may alter the glycosylation pattern of the Fc receptor and thus the binding affinity to Fc. List of a few exemplary polymorphisms in Fc receptor extracellular domains and exemplary Fc receptors constructing ACTR polypeptides are disclosed in WO2020010110A1, WO2020037066A1 and WO2020051493A1.

Table 2 lists a number of exemplary polymorphisms in Fc receptor extracellular domains see, e.g., (Kim et al., Journal of Molecular Evolution, 53(1): 1-9 (2001)) which may be used in any of the methods or constructs described herein:

TABLE 2
Exemplary Polymorphisms in Fc Receptors

| Amino | | | | | | | | | | |
|---|---|---|---|---|---|---|---|---|---|---|
| Acid | | | | | | | | | | |
| Number | 19 | 48 | 65 | 89 | 105 | 130 | 134 | 141 | 142 | 158 |
| FCR10 | R | S | D | I | D | G | F | Y | T | V |
| P08637 | R | S | D | I | D | G | F | Y | I | F |
| S76824 | R | S | D | I | D | G | F | Y | I | V |
| J04162 | R | N | D | V | D | D | F | H | I | V |
| M31936 | S | S | N | I | D | D | F | H | I | V |
| M24854 | S | S | N | I | E | D | S | H | I | V |
| X07934 | R | S | N | I | D | D | F | H | I | V |
| X14356 | N | N | N | S | E | S | S | S | I | I |
| (FcγRII) | | | | | | | | | | |
| M31932 | S | T | N | R | E | A | F | T | I | G |
| (FcγRI) | | | | | | | | | | |
| X06948 | R | S | E | S | Q | S | E | S | I | V |
| (FcεRIα) | | | | | | | | | | |

Fc receptors are classified based on the isotype of the antibody to which it is able to bind. For example, Fc-gamma receptors (FcγR) generally bind to IgG antibodies, such as one or more subtype thereof (i.e., IgG1, IgG2, IgG3, IgG4); Fc-alpha receptors (FcαR) generally bind to IgA antibodies; and Fc-epsilon receptors (FcεR) generally bind to IgE antibodies. In some embodiments, the Fc receptor is an FcγR receptor, an FcαR, or an FcεR. Examples of FcγRs include, without limitation, CD64A, CD64B, CD64C, CD32A, CD32B, CD16A, and CD16B. An example of an FcαRs is FcαR1/CD89. Examples of FcεRs include, without limitation, FcεRI and FcεRII/CD23. Table 3 lists exemplary Fc receptors for use in constructing the ACTR polypeptides described herein and their binding activity to corresponding Fc domains:

TABLE 3
Exemplary Fc Receptors

| | Principal | |
|---|---|---|
| Receptor name | antibody ligand | Affinity for ligand |
| FcγRI (CD64) | IgG1 and IgG3 | High (Kd ~10⁻⁹ M) |
| FcγRIIA (CD32) | IgG | Low (Kd >10⁻⁷ M) |
| FcγRIIB1 (CD32) | IgG | Low (Kd >10⁻⁷ M) |
| FcγRIIB2 (CD32) | IgG | Low (Kd >10⁻⁷ M) |
| FcγRIIIA (CD16a) | IgG | Low (Kd >10⁻⁶ M) |
| FcyRIIIB (CD16b) | IgG | Low (Kd >10⁻⁶ M) |
| FcεRI | IgE | High (Kd ~10⁻¹⁰ M) |
| FcεRII (CD23) | IgE | Low (Kd >10⁻⁷ M) |
| FcαRI (CD89) | IgA | Low (Kd >10⁻⁶ M) |
| Fcα/μR | IgA and IgM | High for IgM, Mid for IgA |
| FcRn | IgG | |

Selection of the ligand binding domain of an FcR for use in the ACTR polypeptides described herein will be apparent to one of skill in the art. For example, it may depend on factors such as the isotype of the antibody to which binding of the Fc receptor is desired and the desired affinity of the binding interaction.

In some examples, the Fc binding domain is the extracellular ligand-binding domain of CD16, which may incorporate a naturally occurring polymorphism that may modulate affinity for Fc. In some examples, the Fc binding domain is the extracellular ligand-binding domain of CD16 incorporating a polymorphism at position 158 (e.g., valine or phenylalanine). In some embodiments, the Fc binding domain is produced under conditions that alter its glycosylation state and its affinity for Fc.

The amino acid sequences of human CD16A F158 and CD16A V158 variants are provided herewith with the F158 and V158 residue in bold/underlined.

CD16A F158 (F158 bold/underlined) (SEQ ID NO: 83):
MWQLLLPTALLLLVSAGMRTEDLPKAVVFLEPQWYRVLEKDSVTLKCQGAYSPEDNS
TQWFHNESLISSQASSYFIDAATVDDSGEYRCQTNLSTLSDPVQLEVHIGWLLLQAP
RWVFKEEDPIHLRCHSWKNTALHKVTYLQNGKGRKYFHHNSDFYIPKATLKDSGSYF
CRGLFGSKNVSSETVNITITQGLAVSTISSFFPPGYQVSFCLVMVLLFAVDTGLYFS
VKTNIRSSTRDWKDHKFKWRKDPQDK
CD16A V158 (V158 bold/underlined) (SEQ ID NO: 84):
MWQLLLPTALLLLVSAGMRTEDLPKAVVELEPQWYRVLEKDSVTLKCQGAYSPEDNS
TQWFHNESLISSQASSYFIDAATVDDSGEYRCQTNLSTLSDPVQLEVHIGWLLLQAP
RWVFKEEDPIHLRCHSWKNTALHKVTYLQNGKGRKYFHHNSDFYIPKATLKDSGSYF
CRGLVGSKNVSSETVNITITQGLAVSTISSFFPPGYQVSFCLVMVLLFAVDTGLYFS
VKTNIRSSTRDWKDHKFKWRKDPQDK

In some embodiments, the Fc binding domain is the extracellular ligand-binding domain of CD16 incorporating modifications that render the ACTR polypeptide specific for a subset of IgG antibodies. For example, mutations that increase or decrease the affinity for an IgG subtype (e.g., IgG1) may be incorporated.

Any of the Fc binding domains described herein may have a suitable binding affinity for the Fc portion of a therapeutic antibody. As used herein, “binding affinity” refers to the apparent association constant or K_{A}. The K_{A} is the reciprocal of the dissociation constant, K_{D}. The extracellular ligand-binding domain of an Fc receptor domain of the ACTR polypeptides described herein may have a binding affinity K_{D} of at least 10⁻⁵, 10⁻⁶, 10⁻⁷, 10⁻⁸, 10⁻⁹, 10⁻¹⁰ M or lower for the Fc portion of antibody. In some embodiments, the Fc binding domain has a high binding affinity for an antibody, isotype(s) of antibodies, or subtype(s) thereof, as compared to the binding affinity of the Fc binding domain to another antibody, isotype(s) of antibodies, or subtypes(s) thereof. In some embodiments, the extracellular ligand-binding domain of an Fc receptor has specificity for an antibody, isotype(s) of antibodies, or subtype(s) thereof, as compared to binding of the extracellular ligand-binding domain of an Fc receptor to another antibody, isotype(s) of antibodies, or subtypes(s) thereof.

Other Fc binding domains as known in the art may also be used in the ACTR constructs described herein including, for example, those described in WO2015/058018A1 and WO2018/140960, the relevant disclosures of each of which are incorporated by reference for the purpose and subject matter referenced herein.

### (ii) Extracellular Antigen Binding Domains

The CAR polypeptides described herein comprise an extracellular antigen binding domain, which re-directs the specificity of immune cells (e.g., NK, αβ T or γδ T cells) expressing the CAR polypeptide. As used herein, “an extracellular antigen binding domain” refers to a peptide or polypeptide having binding specificity to a target antigen of interest, which can be a naturally occurring antigen. Such a target antigen may be any molecule that is associated with a disease or condition, including, but are not limited to, tumor antigens, pathogenic antigens (e.g., bacterial, fungal, or viral), or antigens present on diseased cells, such as those described herein. In some embodiments, the target antigen binding domain targets a native tumor antigen protein. In other embodiments, the target antigen binding domain targets a variant (e.g., mutation) of a tumor antigen protein. Some examples include EGFRvIII scFv recognizes the tumor specific variant of EGFR (Wang, Jiang et al., Cancer Lett, 472:175-180 (2020)).

Non-limiting examples of the extracellular antigen binding domains are tumor antigens, pathogenic antigens and immune cells specific to an autoantigen (Gubin et al., J Clin Invest, 125(9): 3413-3421 (2015); Linnemann et al., Nat Med, 21(1): 81-85 (2015)). Respective diseases and/or conditions to be treated include tumors, inflammatory conditions and auto-immune disorders. In some embodiments, the antigen is selectively expressed or overexpressed on cells of the disease or condition, e.g., the tumor or pathogenic cells, as compared to normal or non-targeted cells or tissues. In some embodiments the extracellular antigen binding domain of binds to a tumor antigen, which is associated with a hematologic or solid tumor.

Non-limiting examples of hematologic tumor extracellular binding domains are domains of CD19, CD20, CD22, Kappa-chain, CD30, CD123, CD33, LeY, CD138, CD5, BCMA, CD7, CD40, ROR1 and IL-1RAP. Non-limiting examples of solid tumor extracellular binding domains are domains of GD2, GPC3, FOLR (e.g., FOLR1 or FOLR2), HER2, EphA2, EFGRVIII, IL13RA2, VEGFR2, ROR1, NKG2D, EpCAM, CEA, Mesothelin, MUC1, CLDN18.2, CD171, CD133, PSCA, cMET, EGFR, PSMA, ROR1, FAP, CD70, MUC16, L1-CAM, B7H3, GUCY2C, Nectin4, LRRC15, PSMA and CAIX. In other instances, tumor antigens are derived from cancers that are characterized by tumor-associated antigen expression, such as HER2 expression. Antigens may include epitopic regions or epitopic peptides derived from genes mutated in tumor cells or from genes transcribed at different levels in tumor cells compared to normal cells (e.g., survivin, mutated Ras, bcr/abl rearrangement, HER2, mutated or wild-type p53).

The extracellular antigen binding domain as described herein does not comprise the Fc portion of an immunoglobulin, and may not bind to an extracellular domain of an Fc receptor. An extracellular domain that does not bind to an FcR means that the binding activity between the two is not detectable using a conventional assay or only background or biologically insignificant binding activity is detected using the conventional assay.

In some instances, the extracellular antigen binding domain of any CAR polypeptides described herein is a peptide or polypeptide capable of binding to a cell surface antigen (e.g., a native and mutated tumor antigen), and may be presented on the cell surface of an antigen-presenting cell.

The extracellular antigen binding domain may be a single-chain antibody fragment (scFv) or a single domain antibody that binds to a tumor antigen, a pathogenic antigen, or an immune cell specific to an autoantigen. These may be derived from an antibody that binds the target cell surface antigen with a high binding affinity. In some embodiments, the extracellular antigen binding domain is a single chain variable fragment (scFv) preferably maintaining the binding properties of the antibody it is derived from. In some embodiments, extracellular antigen binding domain is a single domain antibody preferably maintaining the binding properties of the antibody it is derived from. In exemplary embodiments, the scFv or single domain antibody binds to a tumor antigen, a pathogenic antigen, or an immune cell specific to an autoantigen. Table 4 lists exemplary cell-surface target antigens and exemplary antibodies binding to such.

TABLE 4
Exemplary Cell Surface Target Antigen and Exemplary Antibodies Binding to such

| Exemplary | Exemplary | Exemplary | Exemplary antibodies |
|---|---|---|---|
| target antigens | antibodies | target antigens | and Fc-fusion agents |
| CD137 (4-1BB) | utomilumab | CD74 | milatuzumab |
| Trophoblast glycoprotein | naptumomab | HLA-DR | IMMU-114 |
| (5T4) | estafenatox | | |
| Adenosine A2a receptor | anti-A2aR | Hsp70 | mi-TUMEXtx |
| (A2aR) | mAbs | | |
| Alk-1 protein kinase | ascrinvacumab | Hsp 90 | ZSG-102 |
| (ACVRL1) | | | |
| ADAM-10 (ADAM10) | 8C7 | ICAM-1 | BI-505 |
| TACE (ADAM17) | MEDI-3622 | Inducible T-cell co- | GSK-3359609 |
| | | stimulator (ICOS) | |
| ADAM-28 (ADAM28) | GFC-201 | Immunoglobulin kappa (Ig | KappaMab |
| | | kappa) | |
| CD156; Immunoglobulin | MAB-1031 | Immunoglobulin antigen | LambdaMab |
| G1; Immunoglobulin G2 | | (Ig lambda) | |
| (ADAM8) | | | |
| ADAM-9 (ADAM9) | AEX-6003 | IL-6 receptor (IL-6R) | tocilizumab |
| Anterior gradient protein 2 | agtuzumab | IL-7 receptor (IL-7R) | anti-IL7R mAbs |
| homolog (AGR2) | | | |
| Anaplastic lymphoma | KTN-0125 | IL-13 receptor alpha 1 | ASLAN-004 |
| kinase (ALK) | | subunit (IL13RA1) | |
| Angiopoietin ligand-2 | vanucizumab | IL-13 receptor alpha 2 | anti-IL13RA2 mAbs |
| (Ang-2); Vascular | | subunit (IL13RA2) | |
| endothelial growth factor- | | | |
| A (VEGF-A) | | | |
| Lactadherin (Anti- | TriAb | IL-1 receptor accessory | CAN-04 |
| idiotype) | (11D10) | protein (IL1RAP) | |
| Tumor necrosis factor | BION-1301 | IL-2 receptor beta (IL2R | Mikbeta1 |
| ligand 13 (APRIL) | | beta) | |
| Aspartate beta- | PAN-622 | Immunoglobulin like | BAY-1905254 |
| hydroxylase (ASPH) | | domain receptor 2 (ILDR2) | |
| Axl tyrosine kinase (AXL) | BA-3011 | Integrin alpha-X/beta-1 | anti-Integrin a10b1 |
| | | (Integrin a10b1) | mAbs |
| CD276 antigen (B7-H3) | BVD m276; | Integrin alpha-3/beta-1 | BCMab-1 |
| | hu8H9 | (Integrin a3b1) | |
| V-set domain-containing | FPA-150 | Integrin alpha-6/beta-4 | 90Y-ITGA6B4 |
| T-cell activation inhibitor | | (Integrin a6b4) | |
| 1 (VTCN1; also B7-H4) | | | |
| B-cell activating factor; | blisibimod | Integrin alpha-9 (Integrin | GND-001 |
| (BAFF; also TNFSF13B | | a9) | |
| and CD257) | | | |
| B-cell activating factor | VAY736 | CD49b (Integrin alpha 2) | Vatelizumab |
| receptor; (BAFF-R; also | | | |
| TNFSF13C and CD268) | | | |
| BAG molecular chaperone | anti-BAG3 | CD49c (Integrin alpha 3) | anti-CD49c mAbs |
| regulator 3 (BAG3) | mAbs | | |
| Basigin (BSG; CD147) | cHAb18 | CD49d; (Integrin alpha 4) | anti-CD49d mAbs |
| B-cell maturation antigen | SEA-BCMA | CD51 | abituzumab |
| (BCMA; also TNFRSF17) | | | |
| ADP ribosyl cyclase-2 | OX-001 | CD29 (integrin beta 1) | OS-2966 |
| (BST1) | | | |
| B and T lymphocyte | 40E4 | CD61 (Integrin beta 3) | anti-CD61 mAbs |
| attenuator (BTLA) | | | |
| Complement C5a receptor | neutrazumab | Jagged-1 | anti-Jagged-1 mAbs |
| (C5aR) | | | |
| CACNA2D1 calcium | anti- | Kidney-associated antigen | AB-3A4 |
| channel subunit | CACNA2D1 | 1 (KAAG1) | |
| (CACNA2D1) | mAbs | | |
| Carbonic anhydrase-IX | G250 | Potassium channel | Y-4 |
| (CAIX) | | subfamily K member 9 | |
| | | (KCNK9) | |
| Calreticulin (CALR) | Anti-CALR | KIR2DL1/2L3 | lirilumab |
| | mAbs | | |
| Caveolin 1 (CAV1) | anti-CAV1 | tyrosine-protein kinase kit | CDX-0158 |
| | mAbs | (KIT) | |
| Carbonic anhydrase-XII | 177Lu-6A10- | L1CAM | anti-L1CAM mAbs |
| (CAXII) | Fab; anti- | | |
| | CAXII mAbs | | |
| CCR2 chemokine receptor | plozalizumab | Death receptor 5 (DR5) | APOMAB |
| (CCR2) | | | |
| CCR3 chemokine receptor | anti-CCR3 | CD223 (LAG3) | relatlimab |
| (CCR3) | mAbs | | |
| CCR4 chemokine receptor | mogamulizumab | Lewis Y | hu3S193; MB311 |
| (CCR4) | | | |
| CCR5 chemokine receptor | PRO 140; | Zinc transporter SLC39A6 | SGN-LIV1 |
| (CCR5) | CCR5mAb004 | (LIV1) | |
| CCR7 chemokine receptor | anti-CCR7 | Lysyl oxidase-like protein 2 | AB-0023 |
| (CCR7) | mAbs | (LOXL2) | |
| CCR9 chemokine receptor | anti-CCR9 | Leucine rich repeat- | ABBV-085 |
| (CCR9) | mAbs | containing protein 15 | |
| | | (LRRC15) | |
| Interleukin-3 receptor | CSL362; | Leucine rich repeat- | ARGX-115 |
| alpha (IL3RA; CD123) | KHK2823 | containing protein 32 | |
| | | (LRRC32) | |
| Aminopeptidase N | MI-130110 | Lymphocyte antigen 75 | MEN-1309 |
| (CD13) | | (LY75) | |
| Prominin 1 (CD133) | anti-CD133 | Ly6/PLAUR domain- | BAY-1129980 |
| | mAbs | containing protein 3 | |
| | | (LYPD3) | |
| Syndecan-1 (CD138) | indatuximab | Melanoma associated | LxC-002 |
| | ravtansine | antigen (MAGE peptide | |
| | | presented in MHC) | |
| CD160 | ELB-021 | Matriptase (ST14) | anti-ST14 mAbs |
| Activated leukocyte cell | CX-2009 | MICA/B | IPH4301 |
| adhesion molecule | | | |
| (CD166) | | | |
| B-lymphocyte antigen | MOR208 | MIF/HLA-A2 (MIF peptide | RL21A |
| CD19 | | presented in MHC) | |
| B-lymphocyte antigen | rituximab; | Anti-mullerian hormone II | GM-102 |
| CD20 | obinituzumab; | (MHR2) | |
| | ocaratuzumab | | |
| Membrane glycolprotein | samalizumab | MMP1/HLA (MMP1 | Anti-MMP1/HLA |
| OX2 CD200 | | peptide presented in | mAbs |
| | | MHC1) | |
| CD22 | epratuzumab | Metalloprotease-9 (MMP9) | andecaliximab |
| Immunoglobulin epsilon | lumiliximab | Mesothelin (MSLN) | MORAb-009 |
| Fc receptor II (CD23) | | | |
| Signal transducer CD24 | anti-CD24 | Mucin 1 (MUC1) | PankoMab-GEX |
| | mAbs | | |
| IL-2 receptor alpha | 90Y- | Mucin 13 (MUC13) | anti-MUC13 mAbs |
| subunit CD25 | daclizumab | | |
| CD27 | varilumab | Endomucin (MUC14) | anti-MUC14 mAbs |
| CD28 | theralizumab | Mucin 16 (MUC16) | sofituzumab |
| CD3 | Muromonab- | Cell surface glycoprotein | AA98 |
| | CD3 (OKT3) | MUC18 (CD146) | |
| CD30 | brentuximab | Mucin 5AC (MUC5AC) | ensituximab |
| | vedotin | | |
| Immunoglobulin gamma | BI-1206 | N-glycolyl GM3 | 99mTc-labeled 14F7 |
| Fc receptor IIB (CD32B) | | (NeuGcGM3) | |
| CD33 | lintuzumab | Sodium-dependent | XMT-1536 |
| | | phosphate transport protein | |
| | | 2B (SLC34A2) | |
| CD37 | otlertuzumab | Nucleolin (NCL) | anti-nucleolin mAbs |
| ADP ribosyl cyclase-1 | daratumumab | Nectin-4 | enfortumab vedotin |
| (CD38) | | | |
| CD39 | OREG-103 | Neurofibromin (NF1) | anti-neurofibromin |
| | | | mAbs |
| CD4 | IT-1208 | NGcGM3 ganglioside | racotumomab |
| CD40 | lucatumumab | NKG2A | monalizumab |
| CD43 | leukotuximab | non-POU domain- | PAT-LM1 |
| | | containing octamer-binding | |
| | | protein (NONO) | |
| CD44 | RG7356 | Notch-1 | brontictuzumab |
| CD45 | 131I-BC8 | CD73 | oleclumab |
| Membrane cofactor | AugmAb | Netrin-1 (NTN1) | NP-137 |
| protein (CD46) | | | |
| CD47 | Hu5F9-G4 | OX-40 | PF-04518600 |
| CD52 | alemtuzumab | P2X purinoceptor 7 | BIL-010t |
| | | (P2RX7) | |
| CD55 | PAT-SC1 | FGF receptor (pan FGFR) | MM-161 |
| Neural cell adhesion | IMGN-901 | Integrin (Pan integrin) | NOD201 |
| molecule 1; (CD56) | | | |
| T-cell differentiation | itolizumab | P-cadherin, also cadherin-3 | PCA-062 |
| antigen CD6 | | (CDH3) | |
| CD70 | SGN-70 | Programmed cell death | pembrolizumab |
| | | protein 1 (PD-1) | |
| CD79b | polatuzumab | Programmed cell death | avelumab; Euchloe |
| | vedotin | ligand 1 (PD-L1) | H12 |
| CD8 | anti-CD8 | Programmed cell death | rHIgM12B7 |
| | mAbs | ligand 2 (PD-L2) | |
| CD80 | galiximab | PDGF receptor alpha | olaratumumab |
| | | (PDGFRA) | |
| CD98 | IGN-523 | Placenta specific protein 1 | anti-PLAC1 mAbs |
| | | (PLAC1) | |
| CD99 | NV-103 | PR1/HLA (PR1 peptide in | anti-PR1/HLA mAbs |
| | | MHC) | |
| Cadherin-1 (CDH1) | anti-CDH1 | Prolactin receptor PRLR | ABBV-176 |
| | mAbs | | |
| Cadherin-17 (CDH17) | anti-CDH17 | Phosphatidylserine | anti- |
| | mAbs | | phosphatidylserine |
| | | | mAbs |
| Cadherin 19 (CDH19) | anti-CDH19 | Prostate stem cell antigen | anti-PSCA mAbs |
| | mAbs | (PSCA) | |
| Cadherin-6 (CDH6) | HKT-288 | Glutamate | ATL-101 |
| | | carboxypeptidase II | |
| | | (PSMA) | |
| CD66a (CEACAM1) | CM-24 | Parathyroid hormone- | CAL |
| | | related protein (PTH-rP) | |
| CD66e (CEACAM5) | IMMU-130 | Tyrosine-protein kinase- | cofetuzumab |
| | | like 7 (PTK7) | pelidotin |
| CD66c; CD66e | NEO-201 | Protein tyrosine | PRL3-zumab |
| (CEACAM5/6) | | phosphatase IVA3 | |
| | | (PTP4A3) | |
| Claudin 18 (Claudin 18.2) | IMAB362 | Poliovirus receptor related | COM-701 |
| | | immunoglobulin domain | |
| | | containing (PVRIG) | |
| Claudin 6 | IMAB027 | Receptor activator of | denosumab |
| | | nuclear factor kappa- | |
| | | B ligand (RANKL) | |
| SLAM family member 7 | elotuzumab | Recepteur d'origine nantais | anti-RON mAbs |
| (CS1) | | (RON) | |
| colony stimulating factor- | cabiralizumab | Tyrosine-protein kinase | Cirmtuzumab |
| 1 receptor (CSF1R) | | transmembrane receptor | antibody with |
| | | ROR1 (ROR1); also | CDRs/framework of |
| | | NTRKR1 | SEQ ID NO: 93 |
| Cytotoxic T-lymphocyte | ipilumumab | Tyrosine-protein kinase | BA-3021 |
| protein-4 (CTLA4) | | transmembrane receptor | |
| | | ROR2 (ROR2); also | |
| | | NTRKR2 | |
| Coxsackievirus and | anti-CXADR | R-spondin-3 (RSPO3) | rosmantuzumab |
| adenovirus receptor | mAbs | | |
| (CXADR) | | | |
| CXCR2 chemokine | anti-CXCR2 | Sphingosine-1-phosphate | EDD7H9 |
| receptor | mAbs | receptor 3 (S1PR3) | |
| CXCR3 chemokine | anti-CXCR3 | Surface Antigen In | IGN-786 |
| receptor | mAbs | Leukemia (SAIL) | |
| CXCR4 chemokine | ulocuplumab | Semaphorin-4D | VX-15 |
| receptor | | (SEMA4D) | |
| CXCR5 chemokine | STI-B030X | carbohydrate antigen 19-9 | MVT-1075 |
| receptor | | (CA 19-9) | |
| CXCR7 chemokine | anti-CXCR7 | Sialyl Thomsen nouveau | anti-STn mAbs |
| receptor | mAbs | antigen (STn) | |
| DCLK1 | anti-DCLK1 | Sialic acid-binding Ig-like | AK-002 |
| | mAbs | lectin 8 (Siglec-8) | |
| Dickkopf-related protein 1 | BHQ-880 | Sialic acid-binding Ig-like | anti-Siglec-9 mAbs |
| (DKK1) | | lectin 9 (Siglec-9) | |
| DLK1 | ADCT-701 | Signal Regulatory Protein | OSE-172 |
| | | Alpha (SIRPA) | |
| Delta-like protein ligand 3 | SC16LD6.5 | CD48; also SLAM family | SGN-CD48A |
| (DLL3) | | member 2 (SLAMF2) | |
| Delta-like protein ligand 4 | navicixizumab | CD352; SLAM family | SGN-CD352A |
| (DLL4); VEGF (VEGF) | | member 6 (SLAMF6) | |
| Dipeptidyl peptidase-4 | YSCMA | Neutral amino acid | KM-8094 |
| (DPP4), (also CD26) | | transporter B0 (SLC1A5) | |
| Death receptor-3 (DR3) | PTX-35 | Somatostatin 2 receptor | XmAb-18087 |
| | | (SSTR2) | |
| TRAIL-1 receptor (DR4) | HuYON007 | Stabilin 1 (STAB1) | FP-1305 |
| | MultYbody | | |
| TRAIL-1 receptor; | DR4/DR5 | Metalloreductase | 89Zr-DFO- |
| TRAIL-2 receptor | Surrobody | (STEAP1) | MSTP2109A |
| (DR4/DR5) | | | |
| TRAIL-2 receptor (DR5) | DS-8273 | Survivin | anti-survivin mAbs |
| EGF-like protein 6 | anti-EGFL6 | TAG-72 | 90Y-IDEC-159 |
| (EGFL6) | mAbs | | |
| Epidermal growth factor | cetuximab; | T cell receptor (TCR) | anti-TCR mAbs |
| receptor (EGFR) | Sym004; | | |
| | nimotuzumab | | |
| Epidermal growth factor | ABT-806 | Endosialin (TEM1) | ontuxizumab |
| receptor vIII (EGFRvIII) | | | |
| Epithelial membrane | ONCR-201 | Anthrax toxin receptor 1 | anti-TEM8 mAbs |
| protein 2 (EMP2) | | (ANTXR1); also TEM8 | |
| Endoglin | carotuximab | Tissue factor (TF) | MORAb-066 |
| Ectonucleotide | AGS-16C3F | Transforming growth | anti-TGFBR2 mAbs |
| pyrophosphatase/ | | factor, beta receptor II | |
| phosphodiesterase | | TGF-beta type II | |
| family member 3 (ENPP3) | | (TGFBR2) | |
| Prostaglandin E₂ receptor | anti-PTGER2 | Thomsen-Friedenreich | JAA-F11 |
| 2 (PTGER2) | mAbs | Antigen | |
| Prostaglandin E₂ receptor | anti-PTGER4 | T cell immunoreceptor with | BMS-986207 |
| 4 (PTGER4) | mAbs | Ig and ITIM domains | |
| | | (TIGIT) | |
| EpCAM | oportuzumab | Hepatitis A virus cellular | CDX-014 |
| | monatox | receptor 1 (HAVCR1); also | |
| | | TIM-1 | |
| Ephrin type-A receptor 2 | MEDI-547 | Hepatitis A virus cellular | MBG453 |
| (EphA2) | | receptor 2 (HAVCR2); also | |
| | | TIM-3 | |
| Ephrin type-A receptor 3 | KB004 | Toll-like receptor 2 (TLR- | OPN-305 |
| (EphA3) | | 2) | |
| Fibroblast activation | F19 | Toll-like receptor 4 (TLR- | anti-TLR4 mAbs |
| protein (FAP) | | 4) | |
| CD95 (FAS) | asunercept | Transmembrane 4 L6 | anti-TM4SF1 mAbs |
| | | family member 1 | |
| | | (TM4SF1) | |
| Fc receptor like protein 5 | RG-6160 | Tumor necrosis factor | anti-TNFR2 mAbs |
| (FCRL5) | | receptor 2 (TNFR2) | |
| FGF receptor 1 (FGFR1) | FP-1039 | CD71 | anti-CD71 mAbs |
| FGF receptor 2b | FPA-144 | Triggering receptor | anti-TREM1 mAbs |
| (FGFR2b) | | expressed on myeloid cells | |
| | | 1 (TREM1) | |
| FGF receptor 3 (FGFR3) | B-701 | Tumor-associated calcium | DS-1062 |
| | | signal transducer 2 (Trop-2) | |
| fms-like tyrosine kinase 3 | Flysyn | TWEAK Receptor | MRT-101 |
| (FLT3) | | (TWEAKR) | |
| Folate receptor alpha | farletuzumab; | Tyrosine-protein kinase | ELB-031 |
| (FOLR1) | IMGN853; | receptor TYRO3 (TYRO3) | |
| | KHK2805 | | |
| Folate receptor beta | anti-FOLR | Urokinase receptor (uPAR) | MNPR-101 |
| (FOLR2) | beta mAbs | | |
| Frizzled-1; Frizzled-2; | vantictumab | VEGF-2 (VEGFR2) | ramucirumab |
| Frizzled-5; Frizzled-7; | | | |
| Frizzled-8; (FZD1, 2, 5, 7, 8) | | | |
| Follistatin-like protein 1 | anti-FSTL1 | Vimentin | pritumumab |
| (FSTL1) | mAbs | | |
| Fucosyl-GM1 | BMS-986012 | V-domain Ig suppressor of | JNJ-61610588 |
| | | T cell activation (VISTA) | |
| Frizzled-10 (FZD10) | OTSA-101 | Integrin alpha-4/beta-1 | natalizumab |
| GCSF-R (Also, CD114 | CSL324 | Immunoglobulin iota chain | anti-VPREB1 mAbs |
| and CSFR3) | | (VPREB1) | |
| Galectin 3 binding protein | MP-1959 | Wilms tumor protein | ESK1 |
| (LGALS3) | | (WT1/HLA); WT1 peptide | |
| | | presented in MHC | |
| Guanylate cyclase 2C | TAK-164 | Glypican-3 (GPC3) | codrituzumab |
| (GUCY2C) | | | |
| GD2 | dinutuximab | Transmembrane | CDX-011 |
| | | glycoprotein NMB | |
| | | (GPNMB) | |
| GD3 | PF-06688992 | Leucine-rich repeat- | BNC-101 |
| | | containing G-protein | |
| | | coupled receptor 5 (LGR5) | |
| glucocorticoid-induced | BMS-986156 | family C group 5 member | JNJ-64407564 |
| TNFR-related protein | | G-protein coupled receptor | |
| (GITR) | | D (GPRC5D) | |
| glucocorticoid-induced | EU-102 | Ferritin | Ferritarg P |
| TNFR-related protein | | | |
| ligand (GITRL) | | | |
| premelanocyte protein | anti-PMEL | Erbb2 tyrosine kinase | trastuzumab; |
| (PMEL) | mAbs | (HER2) | pertuzumab; |
| | | | margetuximab |
| Cell surface A33 antigen | Anti-GPA33 | Erbb3 tyrosine kinase | patritumab |
| (GPA33) | mAbs | (HER3) | |
| Glypican-1 (GPC1) | MIL-38 | Globo H | OBI-888 |

The extracellular antigen binding domain may comprise an antigen binding fragment (e.g., an scFv) derived from any of the antibodies listed in Table 4 depending upon the target antigen of interest. In some embodiments, the antigen binding fragment (e.g., an scFv) may comprise the same heavy chain and light chain complementarity determining regions (CDRs) as the antibodies listed in Table 4 depending upon the target antigen of interest. In some examples, the antigen binding fragment (e.g., an scFv) may comprise the same heavy chain variable region (VH) and light chain variable region VL as the antibodies listed in Table 4 depending upon the target antigen of interest.

In other embodiments, the extracellular antigen binding domain of any of the CAR polypeptides described herein may be specific to a pathogenic antigen, such as a bacterial antigen, a viral antigen, or a fungal antigen. Some examples are provided below: influenza virus neuraminidase, hemagglutinin, or M2 protein, human respiratory syncytial virus (RSV) F glycoprotein or G glycoprotein, herpes simplex virus glycoprotein gB, gC, gD, or gE, Chlamydia MOMP or PorB protein, Dengue virus core protein, matrix protein, or glycoprotein E, measles virus hemagglutinin, herpes simplex virus type 2 glycoprotein gB, poliovirus I VP1, envelope glycoproteins of HIV 1, hepatitis B core antigen or surface antigen, diptheria toxin, Streptococcus 24M epitope, Gonococcal pilin, pseudorabies virus g50 (gpD), pseudorabies virus II (gpB), pseudorabies virus III (gpC), pseudorabies virus glycoprotein H, pseudorabies virus glycoprotein E, coronavirus polypeptides, transmissible gastroenteritis glycoprotein 195, transmissible gastroenteritis matrix protein, human papilloma virus E6 or E7, or human hepatitis C virus glycoprotein E1 or E2.

In addition, the extracellular antigen binding domain of the CAR polypeptide described herein may be specific to a tag conjugated to a therapeutic agent, which targets an antigen associated with a disease or disorder (e.g., a tumor antigen or a pathogenic antigen as described herein). In some instances, the tag conjugated to the therapeutic agent can be antigenic and the extracellular antigen binding domain of the CAR polypeptide can be an antigen-binding fragment (e.g., scFv) of an antibody having high binding affinity and/or specificity to the antigenic tag. Exemplary antigenic tags include, but are not limited to, biotin, avidin, a fluorescent molecule (e.g., GFP, YRP, luciferase, or RFP), Myc, Flag, His (e.g., poly His such as 6×His), HA (hemagglutinin), GST, MBP (maltose binding protein), KLH (keyhole limpet hemocyanins), trx, T7, HSV, VSV (e.g., VSV-G), Glu-Glu, V5, e-tag, S-tag, KT3, E2, Au1, Au5, and/or thioredoxin.

In other instances, the tag conjugated to the therapeutic agent is a member of a ligand-receptor pair and the extracellular antigen binding domain comprises the other member of the ligand-receptor pair or a fragment thereof that binds the tag. For example, the tag conjugated to the therapeutic agent can be biotin and the extracellular antigen binding domain of the CAR polypeptide can comprise a biotin-binding fragment of avidin. See, e.g., (Urbanska et al., Cancer Res, 72(7): 1844-1852 (2012); Lohmueller et al., Oncoimmunology, 7(1): e1368604 (2017)). Other examples include anti-Tag CAR, in which the extracellular antigen binding domain is a scFv fragment specific to a protein tag, such as FITC (Tamada et al., Clin Cancer Res, 18(23): 6436-6445 (2012); Kim et al., J Am Chem Soc, 137(8): 2832-2835 (2015); Cao et al., Angew Chem Int Ed Engl, 55(26): 7520-7524 (2016); Ma et al., Proc Natl Acad Sci USA, 113(4): E450-458 (2016)), PNE (Ma, Kim et al., Proc Natl Acad Sci USA, 113(4): E450-458 (2016)), La-SS-B (Cartellieri et al., Blood Cancer J, 6(8): e458 (2016)), Biotin (Lohmueller, Ham et al., Oncoimmunology, 7(1): e1368604 (2017)) and LeucineZipper (Cho et al., Cell, 173(6): 1426-1438.e1411 (2018)). Selection of the antigen binding domain for use in the CAR polypeptides described herein will be apparent to one of skill in the art. For example, it may depend on factors such as the type of target antigen and the desired affinity of the binding interaction.

The extracellular antigen binding domain of any of the CAR polypeptides described herein may have suitable binding affinity for a target antigen (e.g., any one of the targets described herein) or antigenic epitopes thereof. As used herein, “binding affinity” refers to the apparent association constant or K_{A} or the K_{D}. The K_{A} is the reciprocal of the dissociation constant (K_{D}). The extracellular antigen binding domain for use in the CAR polypeptides described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}. Higher affinity binding of an extracellular antigen binding domain for a first antigen relative to a second antigen can be indicated by a higher K_{A} (or a smaller numerical value K_{D}) for binding the first antigen than the K_{A} (or numerical value K_{D}) for binding the second antigen. In such cases, the extracellular antigen binding domain has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 100,000-fold.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH 7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

$$⌊ Bound ⌋ = ⌊ Free ⌋ / ( {K}_{D} + ⌊ Free ⌋ )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

### B. Transmembrane Domain

The transmembrane domain of the chimeric receptor polypeptides (e.g., ACTR polypeptides or CAR polypeptides) described herein can be in any form known in the art. As used herein, a “transmembrane domain” refers to any protein structure that is thermodynamically stable in a cell membrane, preferably a eukaryotic cell membrane. A transmembrane domain compatible for use in the chimeric receptor polypeptides used herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three-dimensional structure of the transmembrane domain. For example, transmembrane domains may form an alpha helix, a complex of more than one alpha helices, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times).

Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-terminus.

In some embodiments, the transmembrane domain of the chimeric receptor polypeptide described herein is derived from a Type I single-pass membrane protein. Preferably, the transmembrane domain is of a membrane protein selected from the group consisting of CD8α, CD8β, 4-1BB/CD137, CD27, CD28, CD34, CD4, FcεRIγ, CD16A, OX40/CD134, CD3ζ, CD38ε, CD3γ, CD3δ, TCRα, TCRβ, TCRγ, CD32, CD64, CD45, CD5, CD9, CD22, CD37, CD80, CD86, CD40, CD40L/CD154, VEGFR2, FAS, FGFR2B, CD2, IL15, IL15R, IL21, DNAM-1, 2B4, NKG2D, NKp44 and NKp46. In some embodiments, the transmembrane domain is from a membrane protein selected from the following: CD8α, CD8b, 4-1BB, CD28, CD34, CD4, FcεRIγ, CD16A, OX40, CD3z, CD3e, CD3g, CD3d, TCRα, CD32, CD64, VEGFR2, FAS, FGFR2B, DNAM-1, 2B4, NKG2D, NKp44 and NKp46. In some examples, the transmembrane domain is of CD8 (e.g., the transmembrane domain is of CD8α). In some examples, the transmembrane domain is of 4-1BB/CD137. In other embodiments, the transmembrane domain is of CD28. In other embodiments, the transmembrane domain is of NKG2D, NKp44 or NKp46. In other examples, the transmembrane domain is of CD34. In yet other examples, the transmembrane domain is not derived from human CD8a. In some embodiments, the transmembrane domain of the chimeric receptor polypeptide is a single-pass alpha helix. The amino acid sequences of exemplary transmembrane domains are provided in Table 5:

TABLE 5
Exemplary Transmembrane Domains

| Transmembrane domain | Sequences | SEQ ID NO: |
|---|---|---|
| CD8α | DIYIWAPLAGTCGVLLLSLVITLYC | 23 |
| 4-1BB/CD137 | DIISFFLALTSTALLFLLFFLTLRFSVV | 24 |
| CD28 | DFWVLVVVGGVLACYSLLVTVAFIIFWVRS | 25 |
| CD28 | FWVLVVVGGVLACYSLLVTVAFIIFWVRS | 26 |
| CD28 | FWVLVVVGGVLACYSLLVTVAFIIFWV | 27 |
| CD34 | DLIALVISGALLAVLGITGYFLMNR | 28 |
| Designed hydrophobic | DLLAALLALLAALLALLAALLARSK | 29 |
| CD4 | DMALIVLGGVAGLLLFIGLGIFFCVR | 30 |
| FCϵRIγ | DLCYILDAILFLYGIVLTLLYCRLK | 31 |
| Designed hydrophobic, | DLLLILLGVLAGVLATLAALLARSK | 32 |
| predicted dimerization | | |
| CD8β | DITLGLLVAGVLVLLVSLGVAIHLC | 33 |
| CD16α | DVSFCLVMVLLFAVDIGLYFSVKIN | 34 |
| OX40/CD134 | DVAAILGLGLVLGLLGPLAILLALY | 35 |
| CD3ϵ | DVMSVATIVIVDICITGGLLLLVYYWSKN | 36 |
| CD3ζ | DLCYLLDGILFIYGVILTALFLRVK | 37 |
| CD3γ | DGFLFAEIVSIFVLAVGVYFIAGQD | 38 |
| CD3δ | DGIIVTDVIATLLLALGVFCFAGHET | 39 |
| TCR-α | DVIGFRILLLKVAGFNLLMTLRLW | 40 |
| CD32 | DIIVAVVIATAVAAIVAAVVALIYCRK | 41 |
| CD64 | DVLFYLAVGIMFLVNTVLWVTIRKE | 42 |
| VEGFR2 | DIIILVGTAVIAMFFWLLLVIILRT | 43 |
| FAS | DLGWLCLLLLPIPLIVWVKRK | 44 |
| FGFR2B | DIAIYCIGVFLIACMVVTVILCRMK | 45 |
| CD8 + 4 aa | FACDIYIWAPLAGTCGVLLLSLVITLYC | 46 |

Transmembrane domains from multi-pass membrane proteins may also be compatible for use in the chimeric receptor polypeptides described herein. Multi-pass membrane proteins may comprise a complex alpha helical structure (e.g., at least 2, 3, 4, 5, 6, 7 or more alpha helices) or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side. In some instances, the reverse orientation of such a native transmembrane protein may be constructed for efficient orientation of the chimeric receptor polypeptide (e.g., CAR) within the immune cell membrane. Either one or multiple helices passes from a multi-pass membrane protein can be used for constructing the chimeric receptor polypeptide described herein.

Transmembrane domains for use in the chimeric receptor polypeptides described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and WO 2000/032776A2, the relevant disclosures of each of which are incorporated by reference herein.

In some embodiments, the amino acid sequence of the transmembrane domain does not comprise cysteine residues. In some embodiments, the amino acid sequence of the transmembrane domain comprises one cysteine residue. In some embodiments, the amino acid sequence of the transmembrane domain comprises two cysteine residues. In some embodiments, the amino acid sequence of the transmembrane domain comprises more than two cysteine residues (e.g., 3, 4, 5, or more).

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence.

The hydropathy, hydrophobic or hydrophilic characteristics of a protein or protein segment, can be assessed by any method known in the art including, for example, the Kyte and Doolittle hydropathy analysis.

### C. Co-Stimulatory Signaling Domains

For many immune cells (e.g., NK or T cells) it is beneficial to include a costimulatory signaling domain for stimulation of an antigen-specific signal, to promote cell proliferation, differentiation and survival, as well as to activate effector functions of the cell. The term “co-stimulatory signaling domain”, as used herein, refers to at least a fragment of a co-stimulatory signaling protein that mediates signal transduction within a cell to induce an immune response such as an effector function (a secondary signal). As known in the art, activation of immune cells such as T cells often require two signals: (1) the antigen specific signal (primary signal) triggered by the engagement of T cell receptor (TCR) and antigenic peptide/MHC complexes presented by antigen presenting cells, which typically is driven by CD3ζ as a component of the TCR complex; and (ii) a co-stimulatory signal (secondary signal) triggered by the interaction between a co-stimulatory receptor and its ligand. A costimulatory receptor transduces a co-stimulatory signal (secondary signal) as an addition to the TCR-triggered signaling and modulates responses mediated by immune cells, such as T cells, NK cells, macrophages, neutrophils, or eosinophils.

Activation of a co-stimulatory signaling domain in an immune cell may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the chimeric receptor polypeptides described herein. The type(s) of co-stimulatory signaling domain is selected based on factors such as the type of the immune cells in which the chimeric receptor polypeptides would be expressed (e.g., T cells, NK cells, macrophages, neutrophils, or eosinophils) and the desired immune effector function (e.g., ADCC). Accordingly, it is in one embodiment that the chimeric receptor polypeptide of the genetically engineered immune cell comprises the at least one co-stimulatory signaling domain. Examples of co-stimulatory signaling domains for use in the chimeric receptor polypeptides may be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PDL2/B7-DC, and PDCD6); members of the TNF superfamily (e.g., 4-1BB/TNFRSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4/CD2525, RELT/TNFRSF19L, TACI/TNFRSF13B, TLIA/TNFSF15, TNF-alpha, and TNF RII/TNFRSFIB); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP10, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2D, NKG2C, NKp30, NKp44, NKp46 and JAMAL. In certain embodiments, the chimeric receptor polypeptides may contain a CD28 co-stimulatory signaling domain or a 4-1BB (CD137) co-stimulatory signaling domain. In some embodiments, at least one co-stimulatory signaling domain is selected from the group consisting of 4-1BB, CD28, CD8α, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL, or any variant thereof.

Also within the scope of the present disclosure are functional variants of any of the co-stimulatory signaling domains described herein, such that the co-stimulatory signaling domain is capable of modulating the immune response of the immune cell. In some embodiments, the co-stimulatory signaling domains comprise up to 10 amino acid residue mutations (e.g., 1, 2, 3, 4, 5, or 8) such as amino acid substitutions, deletions, or additions as compared to a wild-type counterpart. Such co-stimulatory signaling domains comprising one or more amino acid variations (e.g., amino acid substitutions, deletions, or additions) may be referred to as variants.

Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation. For example, mutation of residues 186 and 187 of the native CD28 amino acid sequence may result in an increase in co-stimulatory activity and induction of immune responses by the co-stimulatory domain of the chimeric receptor polypeptide. In some embodiments, the mutations are substitution of a lysine at each of positions 186 and 187 with a glycine residue of the CD28 co-stimulatory domain, referred to as a CD28_{LL-GG} variant. Therefore, a suitable variant of CD28 is the CD28^{LL-GG} variant.

Additional mutations can be made in co-stimulatory signaling domains that may enhance or reduce co-stimulatory activity of the domain will be evident to one of ordinary skill in the art. In some embodiments, the co-stimulatory signaling domain is selected from the group of 4-1BB, CD28, OX40, and CD28_{LL-GG} variant.

In some embodiments, the chimeric receptor polypeptides may contain a single costimulatory domain such as, for example, a CD27 co-stimulatory domain, a CD28 costimulatory domain, a 4-1BB co-stimulatory domain, an ICOS co-stimulatory domain, an OX40 co-stimulatory domain, an OX40L co-stimulatory domain, a 2B4 co-stimulatory domain, a GITR co-stimulatory domain, a NKG2D co-stimulatory domain, a NKp30 costimulatory domain, a NKp44co-stimulatory domain, a NKp46 co-stimulatory domain, a DAP10 co-stimulatory domain, a DAP12 co-stimulatory domain, a DNAM1 co-stimulatory domain, a LFA-1 co-stimulatory domain, a HVEM co-stimulatory domain or a JAMAL costimulatory domain. In one embodiment, the at least one co-stimulatory signaling domain is a CD28 co-stimulatory signaling domain or a 4-1BB co-stimulatory signaling domain.

Selection of the type(s) of co-stimulatory signaling domains may be based on factors such as the type of immune cells (e.g., αβ T, γδ T or NK cells) to be used with the chimeric receptor polypeptides and the desired immune effector function.

In some embodiments, the chimeric receptor polypeptides may comprise more than one co-stimulatory signaling domain (e.g., 2, 3, or more). In one embodiment, the chimeric receptor polypeptide comprises at least two co-stimulatory signaling domains. In one preferred embodiment, the chimeric receptor polypeptide comprises two co-stimulatory signaling domains. In some embodiments, the chimeric receptor polypeptide comprises two or more of the same co-stimulatory signaling domains, for example, two copies of the costimulatory signaling domain of CD28. In some embodiments, the chimeric receptor polypeptide comprises two or more co-stimulatory signaling domains from different costimulatory proteins, such as any two or more co-stimulatory proteins described herein. In some embodiments, the chimeric receptor polypeptide may comprise two or more costimulatory signaling domains from different co-stimulatory receptors, such as any two or more co-stimulatory receptors described herein, for example, CD28 and 4-1BB, CD28 and CD27, CD28 and ICOS, CD28_{LL-GG} variant and 4-1BB, CD28 and OX40, or CD28_{LL-GG} variant and OX40. In some embodiments, the two co-stimulatory signaling domains are CD28 and 4-1BB. In some embodiments, the two co-stimulatory signaling domains are CD28_{LL-GG} variant and 4-1BB. In some embodiments, the two co-stimulatory signaling domains are CD28 and OX40. In some embodiments, the two co-stimulatory signaling domains are CD28_{LL-GG} variant and OX40. In some embodiments, the chimeric receptor polypeptides described herein may contain a combination of a CD28 and ICOSL. In some embodiments, the chimeric receptor polypeptide described herein may contain a combination of CD28 and CD27. In certain embodiments, the 4-1BB co-stimulatory domain is located N-terminal to the CD28 or CD28^{LL-GG} variant co-stimulatory signaling domain.

In some embodiments, one of the co-stimulatory signaling domains is a CD28 costimulatory signaling domain and the other co-stimulatory domain is selected from the group consisting of a CD8α, 4-1BB, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL costimulatory signaling domain. In some embodiments, one of the co-stimulatory signaling domains is a CD8α co-stimulatory signaling domain and the other co-stimulatory domain is selected from the group consisting of a CD28, 4-1BB, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL co-stimulatory signaling domain. In some embodiments, one of the costimulatory signaling domains is a 4-1BB co-stimulatory signaling domain and the other costimulatory domain is selected from the group consisting of a CD8α, CD28, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL co-stimulatory signaling domain.

In some embodiments, the chimeric receptor polypeptides described herein do not comprise a co-stimulatory signaling domain. The amino acid sequences of exemplary costimulatory domains are provided in Table 6.

TABLE 6
Exemplary Co-Stimulatory Domains

| Co-stimulatory | | SEQ ID |
|---|---|---|
| domain | Sequences | NO. |
| 4-1BB/CD137 | KRGRKKLLYIFKQPFMRPVQTTQEEDGCSCRFPEEEEGGCEL | 47 |
| CD28 | RSKRSRLLHSDYMNMTPRRPGPTRKHYQPYAPPRDFAAYRS | 48 |
| OX40/CD134 | ALYLLRRDQRLPPDAHKPPGGGSFRTPIQEEQADAHSTLAKI | 49 |
| OX40L/CD252 | ERVQPLEENVGNAARPRFERNK | 50 |
| ICOS | KKKYSSSVHDPNGEYMFMRAVNTAKKSRLTDVTL | 51 |
| CD27 | QRRKYRSNKGESPVEPAEPCHYSCPREEEGSTIPIQEDYRKPEP | 52 |
| | ACSP | |
| GITR | QLGLHIWQLRSQCMWPRETQLLLEVPPSTEDARSCQFPEEERGE | 53 |
| | RSAEEKGRLGDLWV | |
| HVEM | CVKRRKPRGDVVKVIVSVQRKRQEAEGEATVIEALQAPPDVTTV | 54 |
| | AVEETIPSFTGRSPNH | |
| TIM1 | KKYFFKKEVQQLSVSFSSLQIKALQNAVEKEVQAEDNIYIENSL | 55 |
| | YATD | |
| LFA1/CD11a | DIYIWAPLAGTCGVLLLSLVITLYCYKVGFFKRNLKEKMEAGRG | 56 |
| | VPNGIPAEDSEQLASGQEAGDPGCLKPLHEKDSESGGGKD | |
| CD2 | DIYIWAPLAGTCGVLLLSLVITLYCKRKKQRSRRNDEELETRAH | 57 |
| | RVATEERGRKPHQIPASTPQNPATSQHPPPPPGHRSQAPSHRPP | |
| | PPGHRVQHQPQKRPPAPSGTQVHQQKGPPLPRPRVQPKPPHGAA | |
| | ENSLSPSSN | |
| CD28 + CD27 | RSKRSRLLHSDYMNMTPRRPGPTRKHYQPYAPPRDFAAYRSQR | 58 |
| | RKYRSNKGESPVEPAEPCHYSCPREEEGSTIPIQEDYRKPEPA | |
| | CSP | |
| CD28 + OX40 | RSKRSRLLHSDYMNMTPRRPGPTRKHYQPYAPPRDFAAYRSRR | 59 |
| | DQRLPPDAHKPPGGGSFRTPIQEEQADAHSTLAKI | |
| 4-1BB + CD28 | KRGRKKLLYIFKQPFMRPVQTTQEEDGCSCRFPEEEEGGCELR | 60 |
| | SKRSRLLHSDYMNMTPRRPGPTRKHYQPYAPPRDFAAYRS | |
| CD28 + 4-1BB | RSRLLHSDYMNMTPRRPGPTRKHYQPYAPPRDFAAYRSKRGRK | 61 |
| | KLLYIFKQPFMRPVQTTQEEDGCSCRFPEEEEGGCEL | |
| CD28 + ICOS | RSKRSRLLHSDYMNMTPRRPGPTRKHYQPYAPPRDFAAYRSKK | 62 |
| | KYSSSVHDPNGEYMFMRAVNTAKKSRLTDVTL | |
| CD28_{LL to GG} | RSKRSRGGHSDYMNMTPRRPGPTRKHYQPYAPPRDFAAYRS | 63 |
| variant | | |
| CD28_{LL to GG} | RSKRSRGGHSDYMNMTPRRPGPTRKHYQPYAPPRDFAAYRSKR | 64 |
| variant + 4-1BB | GRKKLLYIFKQPFMRPVQTTQEEDGCSCRFPEEEEGGCEL | |
| DAP10 | CARPRRSPAQEDGKVYINMPGRG | 65 |
| DNAM-1 | NRRRRRERRDLFTESWDTQKAPNNYRSPISTSQPTNQSMDDTR | 66 |
| | EDIYVNYPTFSRRPKTRV | |
| NKp30 | GSTVYYQGKCLTWKGPRRQLPAVVPAPLPPPCGSSAHLLPPVP | 67 |
| | GG | |
| NKp44 | WWGDIWWKIMMELRSLDTQKATCHLQQVTDLPWTSVSSPVERE | 68 |
| | ILYHTVARTKISDDDDEHTL | |

In some instances, the chimeric receptor polypeptide may be free of any costimulatory signaling domain. The optional co-stimulatory signaling domain may be located in the cytoplasm for triggering activation and/or effector signaling.

### D. Cytoplasmic Signaling Domain

Any cytoplasmic signaling domain can be used to create the chimeric receptor polypeptides described herein (e.g., ACTR polypeptides or CAR polypeptides). Such a cytoplasmic domain may be any signaling domain involved in triggering cell signaling (primary signaling) that leads to immune cell proliferation and/or activation. The cytoplasmic signaling domain as described herein is not a co-stimulatory signaling domain, which, as known in the art, relays a co-stimulatory or secondary signal for fully activating immune cells (e.g., CAR-T).

The cytoplasmic signaling domain described herein may comprise an immunoreceptor tyrosine-based activation motif (ITAM) domain (e.g., at least one ITAM domain, at least two ITAM domains, or at least three ITAM domains) or may be ITAM free. An “ITAM,” as used herein, is a conserved protein motif that is generally present in the tail portion of signaling molecules expressed in many immune cells. The motif may comprise two repeats of the amino acid sequence YxxL/I separated by 6-8 amino acids, wherein each x is independently any amino acid, producing the conserved motif YxxL/Ix₍₆₋₈₎YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Examples of ITAMs for use in the chimeric receptor polypeptides comprised within the cytoplasmic signaling domain, without limitation may be: CD3γ, CD3ε, CD3δ, each containing a single ITAM motif while each ζ chain contains 3 distinct ITAM domains (ζa, ζh and ζc). The number and ITAM sequences are also important in the design of CARs (Bettini et al., J Immunol, 199(5): 1555-1560 (2017); Jayaraman et al., EBioMedicine, 58:102931 (2020)).

The amino acid sequence of one exemplary cytoplasmic signaling domain of human CD3ζ is provided below:

(SEQ ID NO: 73)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In some embodiments, the cytoplasmic signaling domain is of CD3ζ or FcεR1γ. In other examples, cytoplasmic signaling domain is not derived from human CD3ζ. In yet other examples, the cytoplasmic signaling domain is not derived from an FcR, when the extracellular Fc-binding domain of the same chimeric receptor polypeptide is derived from CD16A.

In one specific embodiment, several signaling domains can be fused together for additive or synergistic effect. Non-limiting examples of useful additional signaling domains include part or all of one or more of TCRζ chain, CD28, OX40/CD134, 4-1BB/CD137, FcεRIγ, ICOS/CD278, IL2Rβ/CD122, IL-2Rγ/CD132, and CD40.

In other embodiments, the cytoplasmic signaling domain described herein is free of the ITAM motif. Examples include, but are not limited to, the cytoplasmic signaling domain of Jak/STAT, Toll-interleukin receptor (TIR), and tyrosine kinase.

E. Hinge domain

In some embodiments, the chimeric receptor polypeptides (e.g., ACTR polypeptide or CAR polypeptide) described herein further comprise a hinge domain that is located between the extracellular ligand-binding domain and the transmembrane domain. A hinge domain is an amino acid segment that is generally found between two domains of a protein and may allow for flexibility of the protein and movement of one or both domains relative to one another. Any amino acid sequence that provides such flexibility and movement of the extracellular ligand-binding domain relative to the transmembrane domain of the chimeric receptor polypeptide can be used.

Hinge domains of any protein known in the art to comprise a hinge domain are compatible for use in the chimeric receptor polypeptides described herein. In some embodiments, the hinge domain is at least a portion of a hinge domain of a naturally occurring protein and confers flexibility to the chimeric receptor polypeptide. In one embodiment the chimeric receptor polypeptide comprises a hinge domain, which is a hinge domain selected from the list of CD28, CD16A, CD8, IgG, murine CD8α, and DAP12. In some embodiments, the hinge domain is of CD8 (e.g., the hinge domain is of CD8α). In some embodiments, the hinge domain is a portion of the hinge domain of CD8, e.g., a fragment containing at least 15 (e.g., 20, 25, 30, 35, or 40) consecutive amino acids of the hinge domain of CD8. In some embodiments, the hinge domain is of CD28. In some embodiments, the hinge domain is a portion of the hinge domain of CD28, e.g., a fragment containing at least 15 (e.g., 20, 25, 30, 35, or 40) consecutive amino acids of the hinge domain of CD28. The hinge domain and/or the transmembrane domain may be linked to additional amino acids (e.g., 15 aa, 10-aa, 8-aa, 6-aa, or 4-aa) at the N-terminal portion, at the C-terminal portion, or both. Examples can be found, e.g., in (Ying et al., Nat Med, 25(6): 947-953 (2019)).

In some embodiments, the hinge domain is of a CD16A receptor, for example, the whole hinge domain of a CD16A receptor or a portion thereof, which may consist of up to 40 consecutive amino acid residues of the CD16A receptor (e.g., 20, 25, 30, 35, or 40). Such a chimeric receptor polypeptide (e.g., an ACTR polypeptide) may contain no hinge domain from a different receptor (a non-CD16A receptor). In some cases, the chimeric receptor polypeptide described herein may be free of a hinge domain from any non-CD16A receptor. In some instances, such a chimeric receptor polypeptide may be free of any hinge domain.

Hinge domains of IgG antibodies, such as an IgG, IgA, IgM, IgE, or IgD antibodies, are also compatible for use in the chimeric receptor polypeptides described herein. In some embodiments, the hinge domain joins the constant domains CHI and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody, preferably IgG1 and IgG4. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptor polypeptides described herein. In some embodiments, the hinge domain between the C-terminus of the extracellular target-binding domain and the N-terminus of the transmembrane domain is a peptide linker, such as a (GlyₓSer)ₙ linker, wherein x and n, independently can be an integer between 3 and 12, including 3, 4, 5, 6, 7, 8, 9, 10, 11, 12, or more. In some embodiments, the hinge domain is (Gly₄Ser)ₙ, wherein n can be an integer between 3 and 60, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. In certain embodiments, n can be an integer greater than 60. In some embodiments, the hinge domain is (Gly₄Ser)₃ (SEQ ID NO: 15). In some embodiments, the hinge domain is (Gly₄Ser)₆ (SEQ ID NO: 16). In some embodiments, the hinge domain is (Gly₄Ser)₉ (SEQ ID NO: 17). In some embodiments, the hinge domain is (Gly₄Ser)₁₂ (SEQ ID NO: 18). In some embodiments, the hinge domain is (Gly₄Ser)₁₅ (SEQ ID NO: 19). In some embodiments, the hinge domain is (Gly₄Ser)₃₀ (SEQ ID NO: 20). In some embodiments, the hinge domain is (Gly₄Ser)₄₅ (SEQ ID NO: 21). In some embodiments, the hinge domain is (Gly₄Ser)₆₀ (SEQ ID NO: 22).

In other embodiments, the hinge domain is an extended recombinant polypeptide (XTEN), which is an unstructured polypeptide consisting of hydrophilic residues of varying lengths (e.g., 10-80 amino acid residues). Amino acid sequences of XTEN peptides will be evident to one of skill in the art and can be found, for example, in U.S. Pat. No. 8,673,860, the relevant disclosures of which are incorporated by reference herein. In some embodiments, the hinge domain is an XTEN peptide and comprises 60 amino acids. In some embodiments, the hinge domain is an XTEN peptide and comprises 30 amino acids. In some embodiments, the hinge domain is an XTEN peptide and comprises 45 amino acids. In some embodiments, the hinge domain is an XTEN peptide and comprises 15 amino acids.

Any of the hinge domains used for making the chimeric receptor polypeptide as described herein may contain up to 250 amino acid residues. In some instances, the chimeric receptor polypeptide may contain a relatively long hinge domain, for example, containing 150-250 amino acid residues (e.g., 150-180 amino acid residues, 180-200 amino acid residues, or 200-250 amino acid residues). In other instances, the chimeric receptor polypeptide may contain a medium sized hinge domain, which may contain 60-150 amino acid residues (e.g., 60-80, 80-100, 100-120, or 120-150 amino acid residues). In some instances, the hinge domain may be a flexible linker consisting of glycine and serine amino acids having a length between 15 and 60 amino acids, preferably composed of Gly₄Ser units, especially one of the linkers of SEQ ID NO: 16 to SEQ ID NO: 18. Alternatively, the chimeric receptor polypeptide may contain a short hinge domain, which may contain less than 60 amino acid residues (e.g., 1-30 amino acids or 31-60 amino acids). In some embodiments, a chimeric receptor polypeptide (e.g., an ACTR polypeptide) described herein contains no hinge domain or no hinge domain from a non-CD16A receptor. The amino acid sequences of exemplary hinge domains are provided in Table 7:

TABLE 7
Exemplary Hinge Domains

| | | SEQ ID |
|---|---|---|
| Hinge domain | Sequences | NO. |
| CD8α hinge | TTTPAPRPPTPAPTIASQPLSLRPEACRPAAGGAVHIRGLDFA | 4 |
| domain | C | |
| IgG1 (hinge-CH2- | EPKSCDKTHTCPPCPAPELLGGPSVFLFPPKPKDTLMISRIPE | 5 |
| CH3) | VTCVVVDVSHEDPEVKFNWYVDGVEVHNAKTKPREEQYNSTYR | |
| | VVSVLTVLHQDWLNGKEYKCKVSNKALPAPIEKTISKAKGQPR | |
| | EPQVYTLPPSRDELTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPGK | |
| IgG1 (hinge-CH3) | EPKSCDKTHTCPGQPREPQVYTLPPSRDELTKNQVSLTCLVKG | 6 |
| | FYPSDIAVEWESNGQPENNYKTTPPVLDSDGSFFLYSKLTVDK | |
| | SRWQQGNVFSCSVMHEALHNHYTQKSLSLSPGK | |
| IgG1 hinge domain | EPKSCDKTHTCP | 7 |
| IgG4 hinge domain | ESKYGPPCPPCP | 8 |
| CD8α-Fragment 1 | TTTPAPRPPTPAPTIASQPLSLRPEAFAC | 9 |
| (30 aa) | | |
| CD8α-Fragment 2 | TTTPAPRPPTPFAC | 10 |
| (15 aa) | | |
| CD28 | IEVMYPPPYLDNEKSNGTIIHVKGKHLCPSPLFPGPSKP | 11 |
| CD28 (39 aa) | IEVMYPPPYLDNEKSNGTIIHVKGKHLCPSPLFPGPSKP | 12 |
| CD28 (26 aa) | KSNGTIIHVKGKHLCPSPLFPGPSKP | 13 |
| CD28 (16 aa) | GKHLCPSPLFPGPSKP | 14 |
| (Gly₄Ser)₃ (15 aa) | GGGGSGGGGS GGGGS | 15 |
| (Gly₄Ser)₆ (30 aa) | GGGGSGGGGS GGGGSGGGGS GGGGSGGGGS | 16 |
| (Gly₄Ser)₉ (45 aa) | GGGGSGGGGS GGGGSGGGGS GGGGSGGGGS GGGGSGGGGS | 17 |
| | GGGGS | |
| (Gly₄Ser)₁₂ (60 aa) | GGGGSGGGGS GGGGSGGGGS GGGGSGGGGS GGGGSGGGGS | 18 |
| | GGGGSGGGGS GGGGSGGGGS | |
| (Gly₄Ser)₁₅ (75 aa) | GGGGSGGGGS GGGGSGGGGS GGGGSGGGGS GGGGSGGGGS | 19 |
| | GGGGSGGGGS GGGGSGGGGS GGGGSGGGGS GGGGS | |
| (Gly₄Ser)₃₀ (150 aa) | GGGGSGGGGS GGGGSGGGGS GGGGSGGGGS GGGGSGGGGS | 20 |
| | GGGGSGGGGS GGGGSGGGGS GGGGSGGGGS GGGGSGGGGS | |
| | GGGGSGGGGS GGGGSGGGGS GGGGSGGGGS GGGGSGGGGS | |
| | GGGGSGGGGS GGGGSGGGGS GGGGSGGGGS | |
| (Gly₄Scr)₄₅ (225) | GGGGSGGGGS GGGGSGGGGS GGGGSGGGGS GGGGSGGGGS | 21 |
| | GGGGSGGGGS GGGGSGGGGS GGGGSGGGGS GGGGSGGGGS | |
| | GGGGSGGGGS GGGGSGGGGS GGGGSGGGGS GGGGSGGGGS | |
| | GGGGSGGGGS GGGGSGGGGS GGGGSGGGGS GGGGSGGGGS | |
| | GGGGSGGGGS GGGGSGGGGS GGGGSGGGGS GGGGSGGGGS | |
| | GGGGSGGGGS GGGGSGGGGS GGGGS | |
| (Gly₄Ser)₆₀ (240 aa) | GGGGSGGGGS GGGGSGGGGS GGGGSGGGGS GGGGSGGGGS | 22 |
| | GGGGSGGGGS GGGGSGGGGS GGGGSGGGGS GGGGSGGGGS | |
| | GGGGSGGGGS GGGGSGGGGS GGGGSGGGGS GGGGSGGGGS | |
| | GGGGSGGGGS GGGGSGGGGS GGGGSGGGGS GGGGSGGGGS | |
| | GGGGSGGGGS GGGGSGGGGS GGGGSGGGGS GGGGSGGGGS | |
| | GGGGSGGGGS GGGGSGGGGS GGGGSGGGGS GGGGSGGGGS | |
| | GGGGSGGGGS GGGGSGGGGS GGGGSGGGGS GGGGSGGGGS | |
| | GGGGSGGGGS GGGGSGGGGS | |

In some embodiments, chimeric receptor polypeptides described herein may further comprise a hinge domain, which may be located at the C-terminus of the extracellular target binding domain and the N-terminus of the transmembrane domain. The hinge domain may be of any suitable length. In other embodiments, the chimeric receptor polypeptide described herein may have no hinge domain. In yet other embodiments, the chimeric receptor polypeptide described herein may have a shortened hinge domain (e.g., including up to 25 amino acid residues).

### F. Signal Peptide

In some embodiments, the chimeric receptor polypeptide (e.g., ACTR polypeptide or CAR polypeptide) may also comprise a signal peptide (also known as a signal sequence) at the N-terminus of the polypeptide. Preferably, the nucleic acid encoding the chimeric receptor polypeptide is also encoding a signal peptide, whereas in the mature polypeptide the signal peptide has been cleaved off. In general, signal sequences are peptide sequences that target a polypeptide to the desired site in a cell. In some embodiments, the signal sequence targets the chimeric receptor polypeptide to the secretory pathway of the cell and will allow for integration and anchoring of the chimeric receptor polypeptide into the lipid bilayer. Signal sequences including signal sequences of naturally occurring proteins or synthetic, non-naturally occurring signal sequences that are compatible for use in the chimeric receptor polypeptides described herein will be evident to one of skill in the art. In some embodiments, the signal sequence is from CD8α (e.g., SEQ ID NO: 1). In some embodiments, the signal sequence is from CD28 (e.g., SEQ ID NO: 2). In other embodiments, the signal sequence is from the murine kappa chain. In yet other embodiments, the signal sequence is from CD16. See Table 8 below.

TABLE 8
Exemplary Signal Peptides

| Signal Peptide | Sequences | SEQ ID NO. |
|---|---|---|
| CD8a | MALPVTALLLPLALLLHAARP | 1 |
| CD28 | MLRLLLALNLEPSIQVTG | 2 |
| Murine K chain | METDTLLLWVLLLWVPGSTG | 3 |

In some instances, any of the chimeric receptor polypeptides disclosed herein may further comprise a protein tag, examples of which are provided in Table 9 below.

TABLE 9
Exemplary Protein Tags

| Protein Tag | Sequences | SEQ ID NO. |
|---|---|---|
| 2x V5 Tag | GKPIPNPLLGLDSIGKPIPNPLLGLDST | 69 |
| 6xHis Tag | HHHHHH | 70 |
| 2xFlag Tag | DYKDDDDKDYKDDDDK | 71 |
| 3xHA Tag | YPYDVPDYAYPYDVPDYAYPYDVPDYA | 72 |

### G. Examples of ACTR Polypeptides

Exemplary ACTR constructs for use with the methods and compositions described herein may be found, for example, in the instant description and figures or may be found in WO2016/040441A1, WO2017/161333A1, and WO2018/140960A1, each of which is incorporated by reference herein for this purpose. The ACTR polypeptides described herein may comprise a CD16A extracellular domain with binding affinity and specificity for the Fc portion of an IgG molecule, a transmembrane domain, and a CD3ζ cytoplasmic signaling domain. In some embodiments, the ACTR polypeptides may further include one or more costimulatory signaling domains, one of which may be a CD28 co-stimulatory signaling domain or a 4-1BB co-stimulatory signaling domain. The ACTR polypeptides are configured such that, when expressed on an immune cell, the extracellular ligand-binding domain is located extracellularly for binding to a target molecule and the CD3ζ cytoplasmic signaling domain. The co-stimulatory signaling domain may be located in the cytoplasm for triggering activation and/or effector signaling.

In some embodiments, an ACTR polypeptide as described herein may comprise, from N-terminus to C-terminus, the Fc binding domain such as a CD16A extracellular domain, the transmembrane domain, the optional one or more co-stimulatory domains (e.g., a CD28 costimulatory domain, a 4-1BB co-stimulatory signaling domain, an OX40 co-stimulatory signaling domain, a CD27 co-stimulatory signaling domain, or an ICOS co-stimulatory signaling domain), and the CD3ζ cytoplasmic signaling domain.

Alternatively or in addition, the ACTR polypeptides described herein may contain two or more co-stimulatory signaling domains, which may link to each other or be separated by the cytoplasmic signaling domain. The extracellular Fc binder, transmembrane domain, optional co-stimulatory signaling domain(s), and cytoplasmic signaling domain in an ACTR polypeptide may be linked to each other directly, or via a peptide linker. In some embodiments, any of the ACTR polypeptides described herein may comprise a signal sequence at the N-terminus.

Table 10 provides exemplary ACTR polypeptides described herein. These exemplary constructs have, from N-terminus to C-terminus in order, the signal sequence, the Fc binding domain (e.g., an extracellular domain of an Fc receptor), the hinge domain, and the transmembrane, while the positions of the optional co-stimulatory domain and the cytoplasmic signaling domain can be switched.

TABLE 10
Exemplary Components of ACTR polypeptides.

| | | Extracellular | Hinge | Transmem- | Co- | Cytoplasmic |
|---|---|---|---|---|---|---|
| | Signal | domain of Fc | domain | brane | stimulatory | Signaling |
| # | Sequence | receptor (a) | (e) | domain (b) | domain (d) | domain (c) |
| 1 | CD8α | CD16A-V158 | CD8α | CD8α | 4-1BB | CD3ζ |
| 2 | CD8α | CD16A-V158 | CD8α | 4-1BB | 4-1BB | CD3ζ |
| 3 | CD8α | CD16A-V158 | CD8α | CD28 | 4-1BB | CD3ζ |
| 4 | CD8α | CD16A-V158 | CD8α | CD34 | 4-1BB | CD3ζ |
| 5 | CD8α | CD16A-V158 | CD8α | Designed | 4-1BB | CD3ζ |
| | | | | hydrophobic | | |
| 6 | CD8α | CD32A | CD8α | CD8α | 4-1BB | CD3ζ |
| 7 | CD8α | CD16A-V158 | CD8α | CD8α | CD28 | CD3ζ |
| 8 | CD8α | CD16A-V158 | CD8α | CD8α | OX40 | CD3ζ |
| 9 | CD8α | CD16A-V158 | CD8α | CD8α | CD28 + | CD3ζ |
| | | | | | 4-1BB | |
| 10 | CD8α | CD16A-V158 | None | CD8α | 4-1BB | CD3ζ |
| 11 | CD8α | CD16A-V158 | XTEN | CD8α | 4-1BB | CD3ζ |
| 12 | CD8α | CD16A-V158 | CD8α | CD8α | CD28 LL to | CD3ζ |
| | | | | | GG mutant | |
| 13 | CD8α | CD16A-V158 | CD8α | CD8α | CD28 LL to | CD3ζ |
| | | | | | GG mutant + | |
| | | | | | 4-1BB | |
| 14 | CD8α | CD16A-V158 | CD8α | CD4 | 4-1BB | CD3ζ |
| 15 | CD8α | CD16A-V158 | CD8α | CD4 | CD28 LL to | CD3ζ |
| | | | | | GG mutant + | |
| | | | | | 4-1BB | |
| 16 | CD8α | CD16A-V158 | CD8α | FcεRIγ | 4-1BB | CD3ζ |
| 17 | CD8α | CD16A-V158 | CD8α | designed | 4-1BB | CD3ζ |
| | | | | hydrophobic, | | |
| | | | | predicted | | |
| | | | | dimerization | | |
| 18 | CD8α | CD16A-V158 | CD8α | CD8β | 4-1BB | CD3ζ |
| 19 | CD8α | CD16A-V158 | CD8α | C16α | 4-1BB | CD3ζ |
| 20 | CD8α | CD16A-V158 | CD8α | OX40 | 4-1BB | CD3ζ |
| 21 | CD8α | CD16A-V158 | CD8α | CD3ζ | 4-1BB | CD3ζ |
| 22 | CD8α | CD16A-V158 | CD8α | CD3ε | 4-1BB | CD3ζ |
| 23 | CD8α | CD16A-V158 | CD8α | CD3γ | 4-1BB | CD3ζ |
| 24 | CD8α | CD16A-V158 | CD8α | CD3δ | 4-1BB | CD3ζ |
| 25 | CD8α | CD16A-V158 | CD8α | TCR-α | 4-1BB | CD3ζ |
| 26 | CD8α | CD16A-V158 | CD8α | CD32 | 4-1BB | CD3ζ |
| 27 | CD8α | CD16A-V158 | CD8α | CD64 | 4-1BB | CD3ζ |
| 28 | CD8α | CD16A-V158 | CD8α | VEGFR2 | 4-1BB | CD3ζ |
| 29 | CD8α | CD16A-V158 | CD8α | FAS | 4-1BB | CD3ζ |
| 30 | CD8α | CD16A-V158 | CD8α | FGFR2B | 4-1BB | CD3ζ |
| 31 | CD8α | CD16A-F158 | CD8α | CD8α | 4-1BB | CD3ζ |
| 32 | CD8α | CD64A | CD8α | CD8α | 4-1BB | CD3ζ |
| 33 | CD8α | CD16A-V158 | IgG1 (hinge- | CD8α | 4-1BB | CD3ζ |
| | | | CH2-CH3) | | | |
| 34 | CD8α | CD16A-V158 | IgG1 (hinge- | CD8α | 4-1BB | CD3ζ |
| | | | CH3) | | | |
| 35 | CD8α | CD16A-V158 | IgG1 (hinge) | CD8α | 4-1BB | CD3ζ |
| 36 | CD8α | CD16A-V158 | CD8α fragment | CD8α | 4-1BB | CD3ζ |
| | | | 1 (30 aa) | | | |
| 37 | CD8α | CD16A-V158 | CD8α fragment | CD8α | 4-1BB | CD3ζ |
| | | | 2 (15 aa) | | | |
| 38 | CD8α | CD16A-V158 | (Gly₄Ser)₃ | CD8α | 4-1BB | CD3ζ |
| 39 | CD8α | CD16A-V158 | (Gly₄Ser)₆ | CD8α | 4-1BB | CD3ζ |
| 40 | CD8α | CD16A-V158 | (Gly₄Ser)₉ | CD8α | 4-1BB | CD3ζ |
| 41 | CD8α | CD16A-V158 | (Gly₄Ser)₁₂ | CD8α | 4-1BB | CD3ζ |
| 42 | CD8α | CD16A-V158 | XTEN | CD8α | 4-1BB | CD3ζ |
| | | | (60 aa) | | | |
| 43 | CD8α | CD16A-V158 | XTEN | CD8α | 4-1BB | CD3ζ |
| | | | (30 aa) | | | |
| 44 | CD8α | CD16A-V158 | XTEN | CD8α | 4-1BB | CD3ζ |
| | | | (15 aa) | | | |
| 45 | CD28 | CD16A-V158 | CD8α | CD8α | 4-1BB | CD3ζ |
| 46 | Murine κ | CD16A-V158 | CD8α | CD8α | 4-1BB | CD3ζ |
| | chain | | | | | |
| 47 | CD16 | CD16A-V158 | CD8α | CD8α | 4-1BB | CD3ζ |
| 48 | CD8α | CD16A-V158 | CD8α | CD8α | ICOS | CD3ζ |
| 49 | CD8α | CD16A-V158 | CD8α | CD8α | CD27 | CD3ζ |
| 50 | CD8α | CD16A-V158 | CD8α | CD8α | GITR | CD3ζ |
| 51 | CD8α | CD16A-V158 | CD8α | CD8α | HVEM | CD3ζ |
| 52 | CD8α | CD16A-V158 | CD8α | CD8α | TIM1 | CD3ζ |
| 53 | CD8α | CD16A-V158 | CD8α | CD8α | LFA1 | CD3ζ |
| | | | | | (CD11a) | |
| 54 | CD8α | CD16A-V158 | CD8α | CD8α | CD2 | CD3ζ |
| 55 | CD8α | CD16A-V158 | CD8α | FcεR1γ | 4-1BB | FcεR1γ |
| 56 | CD8α | CD16A-V158 | CD8α | CD8α | 4-1BB | FcεR1γ |
| 57 | CD8α | CD16A-V158 | CD28 | CD28 | CD28 | CD3ζ |
| | | | (e.g., 39 aa) | | | |
| 58 | CD8α | CD16A-V158 | none | CD8 | CD28 | CD3ζ |
| 59 | CD8α | CD16A-V158 | CD8 | CD8 | CD28 + | CD3ζ |
| | | | | | CD27 | |
| 60 | CD8α | CD16A-V158 | CD8 | CD8 | CD28 + | CD3ζ |
| | | | | | OX40 | |
| 61 | CD8α | CD16A-V158 | CD8 | CD8 | 4-1BB + | CD3ζ |
| | | | | | CD28 | |
| 62 | CD8α | CD16A-V158 | CD28 | CD28 | CD28 + | CD3ζ |
| | | | | | 4-1BB | |
| 63 | CD8α | CD16A-V158 | CD28 | CD28 | 4-1BB | CD3ζ |
| 64 | CD8α | CD16A-V158 | CD8 | CD8 | CD27 | CD3ζ |
| 65 | CD8α | CD16A-V158 | CD8 | CD8 | CD28 | CD3ζ |
| 66 | CD8α | CD16A-V158 | CD8 | CD8 | ICOS | CD3ζ |
| 67 | CD8α | CD16A-V158 | CD8 | CD8 | OX40 | CD3ζ |
| 68 | CD8α | CD16A-V158 | CD8 | CD8 | CD28 and | CD3ζ |
| | | | | | ICOS | |
| 69 | CD8α | CD16A-V158 | none | CD8 | 4-1BB | CD3ζ |
| 70 | CD8α | CD16A-V158 | none | CD8 | CD27 | CD3ζ |
| 71 | CD8α | CD16A-V158 | none | CD8 | ICOS | CD3ζ |
| 72 | CD8α | CD16A-V158 | none | CD8 | OX40 | CD3ζ |
| 73 | CD8α | CD16A-V158 | none | CD8 + | 4-1BB | CD3ζ |
| | | | | 4aa | | |
| 74 | CD8α | CD16A-V158 | none | CD8 + | CD28 | CD3ζ |
| | | | | 4aa | | |
| 75 | CD8α | CD16A-V158 | CD8 | CD28 | CD28 | CD3ζ |
| 76 | CD8α | CD16A-V158 | CD28 | CD28 | CD28 | CD3ζ |
| | | | (26 aa) | | | |
| 77 | CD8α | CD16A-V158 | CD28 | CD28 | CD28 | CD3ζ |
| | | | (16 aa) | | | |
| 78 | CD8α | CD16A-V158 | none | CD28 | CD28 | CD3ζ |
| 79 | CD8α | CD16A-V158 | CD8 | CD8 | 41BB | CD3ζ |
| 80 | CD8α | CD16A-V158 | CD28 | CD8 | CD28 | CD3ζ |
| | | | (39 aa) | | | |

### H. Examples of CAR Polypeptides

Exemplary CAR polypeptides for use with the methods and compositions described herein may be found, for example, in the instant description and figures or as those known in the art. The CAR polypeptides described herein may comprise an extracellular domain comprising a single-chain antibody fragment (scFv) with binding affinity and specificity for an antigen of interest (e.g., those listed in Table 4), a co-stimulatory domain (e.g., those listed in Table 6) and a CD3ζ cytoplasmic signaling domain. In some embodiments, the CAR polypeptide may further comprise a hinge domain (e.g., those listed in Table 7).

In specific examples, a CAR polypeptide described herein may comprise (i) a CD28 co-stimulatory domain or a 4-1BB co-stimulatory domain; and (ii) a CD28 transmembrane domain, a CD28 hinge domain, or a combination thereof. In further specific examples, a CAR polypeptide described herein may comprise (i) a CD28 co-stimulatory domain or a 4-1BB co-stimulatory domain, (ii) a CD8α transmembrane domain, a CD8α hinge domain, or a combination thereof. In some embodiments, the CAR polypeptides may further include one or more co-stimulatory signaling domains, one of which may be a CD28 co-stimulatory signaling domain or a 4-1BB co-stimulatory signaling domain. In other examples, a CAR polypeptide described herein may comprise (i) a CD28 co-stimulatory domain or a 4-1BB costimulatory domain, (ii) a CD28 transmembrane domain, a CD8α hinge domain, or a combination thereof.

In an exemplary embodiment, the CAR polypeptide comprises (i) a CD8α hinge domain (ii) a CD8α transmembrane domain, (iii) a CD28 co-stimulatory domain or a 4-1BB co-stimulatory domain, (iv) a CD3ζ cytoplasmic signaling domain or a combination thereof. In other embodiments, the CAR polypeptide comprising two co-stimulatory domains further comprises (i) a CD8α or CD28 hinge domain (ii) a CD8α or CD28 transmembrane domain, (iii) a CD28 co-stimulatory domain or a 4-1BB co-stimulatory domain, (iv) a OX40L costimulatory domain, a 2B4 co-stimulatory domain, a DAP10 co-stimulatory domain, a DNAM-1 co-stimulatory domain, a NKG2D co-stimulatory domain, a NKp30 co-stimulatory domain, a NKp44 co-stimulatory domain, a NKp46 co-stimulatory domain or a JAMAL costimulatory domain, or (v) a CD3ζ cytoplasmic signaling domain or a combination thereof. In another exemplary embodiment, the CAR polypeptide comprising two co-stimulatory domains further comprises (i) a CD8α hinge domain (ii) a CD28 transmembrane domain, (iii) a CD28 co-stimulatory domain or a 4-1BB co-stimulatory domain, (iv) a OX40L costimulatory domain, a 2B4 co-stimulatory domain, a DAP10 co-stimulatory domain, a DNAM-1 co-stimulatory or a JAMAL co-stimulatory domain, or (v) a CD3ζ cytoplasmic signaling domain or a combination thereof.

In another exemplary embodiment, the CAR polypeptide comprising two costimulatory domains further comprises (i) a CD8α hinge domain (ii) a CD28 transmembrane domain, a NKp44 transmembrane domain, a NKG2D transmembrane domain or a NKp46 transmembrane domain, (iii) a CD28 co-stimulatory domain, a 4-1BB co-stimulatory domain, a 2B4 co-stimulatory domain or a DAP10 co-stimulatory, (iv) an OX40L co-stimulatory domain, a 2B4 co-stimulatory domain, a DAP10 co-stimulatory domain, a DAP12 costimulatory domain, a DNAM-1 co-stimulatory domain or a JAMAL co-stimulatory domain, or (v) a CD3ζ cytoplasmic signaling domain, a DAP12 cytoplasmic signaling domain or a 2B4 cytoplasmic signaling domain or a combination thereof. In an exemplary embodiment, the CAR polypeptide comprises (i) a CD8α hinge domain (ii) a CD28 transmembrane domain, (iii) a CD28 co-stimulatory domain or a 4-1BB co-stimulatory domain, (iv) an OX40L co-stimulatory domain or an OX40 co-stimulatory domain, (v) a CD3ζ cytoplasmic signaling domain or a combination thereof.

For example, the CAR polypeptide may comprise an amino acid sequence selected SEQ ID NO: 86, SEQ ID NO: 87 or SEQ ID NO: 94 provided below.

The CAR polypeptides are configured such that, when expressed on an immune cell (e.g., T or NK cell), the extracellular antigen-binding domain is located extracellularly for binding to a target molecule (e.g., a tumor antigen) and the CD3ζ cytoplasmic signaling domain is located intracellularly for signaling into the cell. The co-stimulatory signaling domain may be located in the cytoplasm for triggering activation and/or effector signaling.

In some embodiments, a CAR polypeptide as described herein may comprise, from N-terminus to C-terminus, the extracellular antigen binding domain, the transmembrane domain, the optional one or more co-stimulatory domains (e.g., a CD28 co-stimulatory domain, a 4-1BB co-stimulatory signaling domain, an OX40L co-stimulatory signaling domain, an OX40 co-stimulatory signaling domain, a CD27 co-stimulatory signaling domain, a 2B4 co-stimulatory signaling domain or an ICOS co-stimulatory signaling domain), and the CD3ζ cytoplasmic signaling domain.

Alternatively or in addition, the CAR polypeptides described herein may contain two or more co-stimulatory signaling domains, which may link to each other or be separated by the cytoplasmic signaling domain. The extracellular antigen binding domain, transmembrane domain, optional co-stimulatory signaling domain(s), and cytoplasmic signaling domain in a CAR polypeptide may be linked to each other directly, or via a peptide linker. In some embodiments, any of the CAR polypeptides described herein may comprise a signal sequence at the N-terminus.

Table 11 to Table 13 provide exemplary CAR polypeptides for CAR-αβ T cells, CAR NK cells and CAR γδ T cells described herein. These exemplary constructs have, from N-terminus to C-terminus in order, the signal sequence, the antigen binding domain (e.g., an scFv fragment targeting an antigen such as a tumor antigen or a pathogenic antigen), the hinge domain, and the transmembrane, while the positions of the optional co-stimulatory domain(s) and the cytoplasmic signaling domain can be switched.

TABLE 11
Exemplary examples of CAR-αβ T cell constructs

| | | Antigen | Hinge | Transmem- | Co-stim. | Co-stim. | Cytoplasmic |
|---|---|---|---|---|---|---|---|
| | Signal | binding | domain | brane | domain | domain | signaling |
| CAR# | Sequence | domain | (e) | domain (b) | (d1) | (d2) | domain (c) |
| 1 | CD8α | scFv | CD8α | CD8α | 4-1BB | none | CD3ζ |
| 2 | CD8α | scFv | CD8α | CD28 | 4-1BB | none | CD3ζ |
| 3 | CD8α | scFv | CD8α | 4-1BB | 4-1BB | none | CD3ζ |
| 4 | CD8α | scFv | CD8α | CD4 | 4-1BB | none | CD3ζ |
| 5 | CD8α | scFv | CD8α | FcεRIγ | 4-1BB | none | CD3ζ |
| 6 | CD8α | scFv | CD8α | CD8β | 4-1BB | none | CD3ζ |
| 7 | CD8α | scFv | CD8α | C16α | 4-1BB | none | CD3ζ |
| 8 | CD8α | scFv | CD8α | OX40 | 4-1BB | none | CD3ζ |
| 9 | CD8α | scFv | CD8α | CD3ζ | 4-1BB | none | CD3ζ |
| 10 | CD8α | scFv | CD8α | CD3ε | 4-1BB | none | CD3ζ |
| 11 | CD8α | scFv | CD8α | CD3γ | 4-1BB | none | CD3ζ |
| 12 | CD8α | scFv | CD8α | CD3δ | 4-1BB | none | CD3ζ |
| 13 | CD8α | scFv | CD8α | TCR-α | 4-1BB | none | CD3ζ |
| 14 | CD8α | scFv | CD8α | CD32 | 4-1BB | none | CD3ζ |
| 15 | CD8α | scFv | CD8α | CD64 | 4-1BB | none | CD3ζ |
| 16 | CD8α | scFv | CD8α | VEGFR2 | 4-1BB | none | CD3ζ |
| 17 | CD8α | scFv | CD8α | FAS | 4-1BB | none | CD3ζ |
| 18 | CD8α | scFv | CD8α | FGFR2B | 4-1BB | none | CD3ζ |
| 19 | CD8α | scFv | CD8α | CD8α | CD28 | none | CD3ζ |
| 20 | CD8α | scFv | CD8α | CD8α | 4-1BB | none | CD3ζ |
| 21 | CD8α | scFv | CD8α | CD8α | ICOS | none | CD3ζ |
| 22 | CD8α | scFv | CD8α | CD8α | CD27 | none | CD3ζ |
| 23 | CD8α | scFv | CD8α | CD8α | GITR | none | CD3ζ |
| 24 | CD8α | scFv | CD8α | CD8α | HVEM | none | CD3ζ |
| 25 | CD8α | scFv | CD8α | CD8α | TIM1 | none | CD3ζ |
| 26 | CD8α | scFv | CD8α | CD8α | LFA1 | none | CD3ζ |
| 27 | CD8α | scFv | CD8α | CD8α | CD2 | none | CD3ζ |
| 28 | CD8α | scFv | CD8α | CD8α | OX40 | none | CD3ζ |
| 29 | CD8α | scFv | CD8α | CD8α | OX40L | none | CD3ζ |
| 30 | CD8α | scFv | CD8α | CD28 | CD28 | none | CD3ζ |
| 31 | CD8α | scFv | CD8α | CD28 | 4-1BB | none | CD3ζ |
| 32 | CD8α | scFv | CD8α | CD28 | ICOS | none | CD3ζ |
| 33 | CD8α | scFv | CD8α | CD28 | CD27 | none | CD3ζ |
| 34 | CD8α | scFv | CD8α | CD28 | GITR | none | CD3ζ |
| 35 | CD8α | scFv | CD8α | CD28 | HVEM | none | CD3ζ |
| 36 | CD8α | scFv | CD8α | CD28 | TIM1 | none | CD3ζ |
| 37 | CD8α | scFv | CD8α | CD28 | LFA1 | none | CD3ζ |
| 38 | CD8α | scFv | CD8α | CD28 | CD2 | none | CD3ζ |
| 39 | CD8α | scFv | CD8α | CD28 | OX40 | none | CD3ζ |
| 40 | CD8α | scFv | CD8α | CD28 | OX40L | none | CD3ζ |
| 41 | CD8α | scFv | CD8α | 4-1BB | CD28 | none | CD3ζ |
| 42 | CD8α | scFv | CD8α | 4-1BB | 4-1BB | none | CD3ζ |
| 43 | CD8α | scFv | CD8α | 4-1BB | ICOS | none | CD3ζ |
| 44 | CD8α | scFv | CD8α | 4-1BB | CD27 | none | CD3ζ |
| 45 | CD8α | scFv | CD8α | 4-1BB | GITR | none | CD3ζ |
| 46 | CD8α | scFv | CD8α | 4-1BB | HVEM | none | CD3ζ |
| 47 | CD8α | scFv | CD8α | 4-1BB | TIM1 | none | CD3ζ |
| 48 | CD8α | scFv | CD8α | 4-1BB | LFA1 | none | CD3ζ |
| 49 | CD8α | scFv | CD8α | 4-1BB | CD2 | none | CD3ζ |
| 50 | CD8α | scFv | CD8α | 4-1BB | OX40 | none | CD3ζ |
| 51 | CD8α | scFv | CD8α | 4-1BB | OX40L | none | CD3ζ |
| 52 | CD8α | scFv | CD8α | CD8α | CD28 | 4-1BB | CD3ζ |
| 53 | CD8α | scFv | CD8α | CD8α | CD28 | CD27 | CD3ζ |
| 54 | CD8α | scFv | CD8α | CD8α | CD28 | OX40 | CD3ζ |
| 55 | CD8α | scFv | CD8α | CD8α | CD28 | ICOS | CD3ζ |
| 56 | CD8α | scFv | CD8α | CD8α | CD28 | OX40L | CD3ζ |
| 57 | CD8α | scFv | CD8α | CD8α | 4-1BB | CD27 | CD3ζ |
| 58 | CD8α | scFv | CD8α | CD8α | 4-1BB | OX40 | CD3ζ |
| 59 | CD8α | scFv | CD8α | CD8α | 4-1BB | ICOS | CD3ζ |
| 60 | CD8α | scFv | CD8α | CD8α | 4-1BB | OX40L | CD3ζ |
| 61 | CD8α | scFv | CD8α | CD28 | CD28 | 4-1BB | CD3ζ |
| 62 | CD8α | scFv | CD8α | CD28 | CD28 | CD27 | CD3ζ |
| 63 | CD8α | scFv | CD8α | CD28 | CD28 | OX40 | CD3ζ |
| 64 | CD8α | scFv | CD8α | CD28 | CD28 | ICOS | CD3ζ |
| 65 | CD8α | scFv | CD8α | CD28 | CD28 | OX40L | CD3ζ |
| 66 | CD8α | scFv | CD8α | CD28 | 4-1BB | CD27 | CD3ζ |
| 67 | CD8α | scFv | CD8α | CD28 | 4-1BB | OX40 | CD3ζ |
| 68 | CD8α | scFv | CD8α | CD28 | 4-1BB | ICOS | CD3ζ |
| 69 | CD8α | scFv | CD8α | CD28 | 4-1BB | OX40L | CD3ζ |
| 70 | CD8α | scFv | CD8α | 4-1BB | CD28 | 4-1BB | CD3ζ |
| 71 | CD8α | scFv | CD8α | 4-1BB | CD28 | CD27 | CD3ζ |
| 72 | CD8α | scFv | CD8α | 4-1BB | CD28 | OX40 | CD3ζ |
| 73 | CD8α | scFv | CD8α | 4-1BB | CD28 | ICOS | CD3ζ |
| 74 | CD8α | scFv | CD8α | 4-1BB | CD32 | OX40L | CD3ζ |
| 75 | CD8α | scFv | CD8α | 4-1BB | 4-1BB | CD27 | CD3ζ |
| 76 | CD8α | scFv | CD8α | 4-1BB | 4-1BB | OX40 | CD3ζ |
| 77 | CD8α | scFv | CD8α | 4-1BB | 4-1BB | ICOS | CD3ζ |
| 78 | CD8α | scFv | CD8α | 4-1BB | 4-1BB | OX40L | CD3ζ |

TABLE 12
Exemplary examples of CAR-NK cell constructs

| | | Antigen | Hinge | Transmem- | Co-stim. | Co-stim. | Cytoplasmic |
|---|---|---|---|---|---|---|---|
| | Signal | binding | domain | brane | domain | domain | Signaling |
| CAR # | Sequence | domain | (e) | domain (b) | (d1) | (d2) | domain (c) |
| 1 | CD8α | scFv | CD8α | CD8α | 4-1BB | none | CD3ζ |
| 2 | CD8α | scFv | CD8α | CD8β | 4-1BB | none | CD3ζ |
| 3 | CD8α | scFv | CD8α | CD28 | 4-1BB | none | CD3ζ |
| 4 | CD8α | scFv | CD8α | 4-1BB | 4-1BB | none | CD3ζ |
| 5 | CD8α | scFv | CD8α | CD4 | 4-1BB | none | CD3ζ |
| 6 | CD8α | scFv | CD8α | FcεRIγ | 4-1BB | none | CD3ζ |
| 7 | CD8α | scFv | CD8α | CD8β | 4-1BB | none | CD3ζ |
| 8 | CD8α | scFv | CD8α | C16α | 4-1BB | none | CD3ζ |
| 9 | CD8α | scFv | CD8α | OX40 | 4-1BB | none | CD3ζ |
| 10 | CD8α | scFv | CD8α | CD3ζ | 4-1BB | none | CD3ζ |
| 11 | CD8α | scFv | CD8α | CD3ε | 4-1BB | none | CD3ζ |
| 12 | CD8α | scFv | CD8α | CD3γ | 4-1BB | none | CD3ζ |
| 13 | CD8α | scFv | CD8α | CD3δ | 4-1BB | none | CD3ζ |
| 14 | CD8α | scFv | CD8α | TCR-α | 4-1BB | none | CD3ζ |
| 15 | CD8α | scFv | CD8α | CD32 | 4-1BB | none | CD3ζ |
| 16 | CD8α | scFv | CD8α | CD64 | 4-1BB | none | CD3ζ |
| 17 | CD8α | scFv | CD8α | VEGFR2 | 4-1BB | none | CD3ζ |
| 18 | CD8α | scFv | CD8α | FAS | 4-1BB | none | CD3ζ |
| 19 | CD8α | scFv | CD8α | FGFR2B | 4-1BB | none | CD3ζ |
| 20 | CD8α | scFv | CD8α | NKG2D | 4-1BB | none | CD3ζ |
| 21 | CD8α | scFv | CD8α | NKp44 | 4-1BB | none | CD3ζ |
| 22 | CD8α | scFv | CD8α | NKp46 | 4-1BB | none | CD3ζ |
| 23 | CD8α | scFv | CD8α | CD8α | CD28 | none | CD3ζ |
| 24 | CD8α | scFv | CD8α | CD8α | 4-1BB | none | CD3ζ |
| 25 | CD8α | scFv | CD8α | CD8α | DAP10 | none | CD3ζ |
| 26 | CD8α | scFv | CD8α | CD8α | DAP12 | none | CD3ζ |
| 27 | CD8α | scFv | CD8α | CD8α | 2B4 | none | CD3ζ |
| 28 | CD8α | scFv | CD8α | CD8α | CD27 | none | CD3ζ |
| 29 | CD8α | scFv | CD8α | CD8α | CD2 | none | CD3ζ |
| 30 | CD8α | scFv | CD8α | CD8α | OX40 | none | CD3ζ |
| 31 | CD8α | scFv | CD8α | CD8α | OX40L | none | CD3ζ |
| 32 | CD8α | scFv | CD8α | CD8α | DNAM-1 | none | CD3ζ |
| 33 | CD8α | scFv | CD8α | CD8α | NKp30 | none | CD3ζ |
| 34 | CD8α | scFv | CD8α | CD8α | NKp44 | none | CD3ζ |
| 35 | CD8α | scFv | CD8α | CD8α | NKp46 | none | CD3ζ |
| 36 | CD8α | scFv | CD8α | CD8α | ICOS | none | CD3ζ |
| 37 | CD8α | scFv | CD8α | CD8α | NKG2D | none | CD3ζ |
| 38 | CD8α | scFv | CD8α | CD8α | JAMAL | none | CD3ζ |
| 39 | CD8α | scFv | CD8α | CD8α | GITR | none | CD3ζ |
| 40 | CD8α | scFv | CD8α | CD8α | HVEM | none | CD3ζ |
| 41 | CD8α | scFv | CD8α | CD8α | TIM1 | none | CD3ζ |
| 42 | CD8α | scFv | CD8α | CD8α | LFA1 | none | CD3ζ |
| 43 | CD8α | scFv | CD8α | CD28 | CD28 | none | CD3ζ |
| 44 | CD8α | scFv | CD8α | CD28 | 4-1BB | none | CD3ζ |
| 45 | CD8α | scFv | CD8α | CD28 | DAP10 | none | CD3ζ |
| 46 | CD8α | scFv | CD8α | CD28 | DAP12 | none | CD3ζ |
| 47 | CD8α | scFv | CD8α | CD28 | 2B4 | none | CD3ζ |
| 48 | CD8α | scFv | CD8α | CD28 | CD27 | none | CD3ζ |
| 49 | CD8α | scFv | CD8α | CD28 | CD2 | none | CD3ζ |
| 50 | CD8α | scFv | CD8α | CD28 | OX40 | none | CD3ζ |
| 51 | CD8α | scFv | CD8α | CD28 | OX40L | none | CD3ζ |
| 52 | CD8α | scFv | CD8α | CD28 | DNAM-1 | none | CD3ζ |
| 53 | CD8α | scFv | CD8α | CD28 | NKp30 | none | CD3ζ |
| 54 | CD8α | scFv | CD8α | CD28 | NKp44 | none | CD3ζ |
| 55 | CD8α | scFv | CD8α | CD28 | NKp46 | none | CD3ζ |
| 56 | CD8α | scFv | CD8α | CD28 | ICOS | none | CD3ζ |
| 57 | CD8α | scFv | CD8α | CD28 | NKG2D | none | CD3ζ |
| 58 | CD8α | scFv | CD8α | CD28 | JAMAL | none | CD3ζ |
| 59 | CD8α | scFv | CD8α | CD28 | GITR | none | CD3ζ |
| 60 | CD8α | scFv | CD8α | CD28 | HVEM | none | CD3ζ |
| 61 | CD8α | scFv | CD8α | CD28 | TIM1 | none | CD3ζ |
| 62 | CD8α | scFv | CD8α | CD28 | LFA1 | none | CD3ζ |
| 63 | CD8α | scFv | CD8α | 4-1BB | CD28 | none | CD3ζ |
| 64 | CD8α | scFv | CD8α | 4-1BB | 4-1BB | none | CD3ζ |
| 65 | CD8α | scFv | CD8α | 4-1BB | DAP10 | none | CD3ζ |
| 66 | CD8α | scFv | CD8α | 4-1BB | DAP12 | none | CD3ζ |
| 67 | CD8α | scFv | CD8α | 4-1BB | 2B4 | none | CD3ζ |
| 68 | CD8α | scFv | CD8α | 4-1BB | CD27 | none | CD3ζ |
| 69 | CD8α | scFv | CD8α | 4-1BB | CD2 | none | CD3ζ |
| 70 | CD8α | scFv | CD8α | 4-1BB | OX40 | none | CD3ζ |
| 71 | CD8α | scFv | CD8α | 4-1BB | OX40L | none | CD3ζ |
| 72 | CD8α | scFv | CD8α | 4-1BB | DNAM-1 | none | CD3ζ |
| 73 | CD8α | scFv | CD8α | 4-1BB | NKp30 | none | CD3ζ |
| 74 | CD8α | scFv | CD8α | 4-1BB | NKp44 | none | CD3ζ |
| 75 | CD8α | scFv | CD8α | 4-1BB | NKp46 | none | CD3ζ |
| 76 | CD8α | scFv | CD8α | 4-1BB | ICOS | none | CD3ζ |
| 77 | CD8α | scFv | CD8α | 4-1BB | NKG2D | none | CD3ζ |
| 78 | CD8α | scFv | CD8α | 4-1BB | JAMAL | none | CD3ζ |
| 79 | CD8α | scFv | CD8α | 4-1BB | GITR | none | CD3ζ |
| 80 | CD8α | scFv | CD8α | 4-1BB | HVEM | none | CD3ζ |
| 81 | CD8α | scFv | CD8α | 4-1BB | TIM1 | none | CD3ζ |
| 82 | CD8α | scFv | CD8α | 4-1BB | LFA1 | none | CD3ζ |
| 83 | CD8α | scFv | CD8α | CD8α | CD28 | 4-1BB | CD3ζ |
| 84 | CD8α | scFv | CD8α | CD8α | CD28 | DAP10 | CD3ζ |
| 85 | CD8α | scFv | CD8α | CD8α | CD28 | DAP12 | CD3ζ |
| 86 | CD8α | scFv | CD8α | CD8α | CD28 | CD27 | CD3ζ |
| 87 | CD8α | scFv | CD8α | CD8α | CD28 | CD2 | CD3ζ |
| 88 | CD8α | scFv | CD8α | CD8α | CD28 | OX40 | CD3ζ |
| 89 | CD8α | scFv | CD8α | CD8α | CD28 | OX40L | CD3ζ |
| 90 | CD8α | scFv | CD8α | CD8α | CD28 | ICOS | CD3ζ |
| 91 | CD8α | scFv | CD8α | CD8α | CD28 | DNAM-1 | CD3ζ |
| 92 | CD8α | scFv | CD8α | CD8α | CD28 | NKp30 | CD3ζ |
| 93 | CD8α | scFv | CD8α | CD8α | CD28 | NKp44 | CD3ζ |
| 94 | CD8α | scFv | CD8α | CD8α | CD28 | NKp46 | CD3ζ |
| 95 | CD8α | scFv | CD8α | CD8α | CD28 | NKG2D | CD3ζ |
| 96 | CD8α | scFv | CD8α | CD8α | CD28 | JAMAL | CD3ζ |
| 97 | CD8α | scFv | CD8α | CD8α | CD28 | 2B4 | CD3ζ |
| 98 | CD8α | scFv | CD8α | CD8α | 4-1BB | CD28 | CD3ζ |
| 99 | CD8α | scFv | CD8α | CD8α | 4-1BB | DAP10 | CD3ζ |
| 100 | CD8α | scFv | CD8α | CD8α | 4-1BB | DAP12 | CD3ζ |
| 101 | CD8α | scFv | CD8α | CD8α | 4-1BB | CD27 | CD3ζ |
| 102 | CD8α | scFv | CD8α | CD8α | 4-1BB | CD2 | CD3ζ |
| 103 | CD8α | scFv | CD8α | CD8α | 4-1BB | OX40 | CD3ζ |
| 104 | CD8α | scFv | CD8α | CD8α | 4-1BB | OX40L | CD3ζ |
| 105 | CD8α | scFv | CD8α | CD8α | 4-1BB | ICOS | CD3ζ |
| 106 | CD8α | scFv | CD8α | CD8α | 4-1BB | DNAM-1 | CD3ζ |
| 107 | CD8α | scFv | CD8α | CD8α | 4-1BB | NKp30 | CD3ζ |
| 108 | CD8α | scFv | CD8α | CD8α | 4-1BB | NKp44 | CD3ζ |
| 109 | CD8α | scFv | CD8α | CD8α | 4-1BB | NKp46 | CD3ζ |
| 110 | CD8α | scFv | CD8α | CD8α | 4-1BB | NKG2D | CD3ζ |
| 111 | CD8α | scFv | CD8α | CD8α | 4-1BB | JAMAL | CD3ζ |
| 112 | CD8α | scFv | CD8α | CD8α | 4-1BB | 2B4 | CD3ζ |
| 113 | CD8α | scFv | CD8α | CD8α | 2B4 | 4-1BB | CD3ζ |
| 114 | CD8α | scFv | CD8α | CD8α | 2B4 | CD28 | CD3ζ |
| 115 | CD8α | scFv | CD8α | CD8α | 2B4 | DAP10 | CD3ζ |
| 116 | CD8α | scFv | CD8α | CD8α | 2B4 | DAP12 | CD3ζ |
| 117 | CD8α | scFv | CD8α | CD8α | 2B4 | CD27 | CD3ζ |
| 118 | CD8α | scFv | CD8α | CD8α | 2B4 | CD2 | CD3ζ |
| 119 | CD8α | scFv | CD8α | CD8α | 2B4 | OX40 | CD3ζ |
| 120 | CD8α | scFv | CD8α | CD8α | 2B4 | OX40L | CD3ζ |
| 121 | CD8α | scFv | CD8α | CD8α | 2B4 | ICOS | CD3ζ |
| 122 | CD8α | scFv | CD8α | CD8α | 2B4 | DNAM-1 | CD3ζ |
| 123 | CD8α | scFv | CD8α | CD8α | 2B4 | NKp30 | CD3ζ |
| 124 | CD8α | scFv | CD8α | CD8α | 2B4 | NKp44 | CD3ζ |
| 125 | CD8α | scFv | CD8α | CD8α | 2B4 | NKp46 | CD3ζ |
| 126 | CD8α | scFv | CD8α | CD8α | 2B4 | NKG2D | CD3ζ |
| 127 | CD8α | scFv | CD8α | CD8α | 2B4 | JAMAL | CD3ζ |
| 128 | CD8α | scFv | CD8α | CD28 | CD28 | 4-1BB | CD3ζ |
| 129 | CD8α | scFv | CD8α | CD28 | CD28 | DAP10 | CD3ζ |
| 130 | CD8α | scFv | CD8α | CD28 | CD28 | DAP12 | CD3ζ |
| 131 | CD8α | scFv | CD8α | CD28 | CD28 | CD27 | CD3ζ |
| 132 | CD8α | scFv | CD8α | CD28 | CD28 | CD2 | CD3ζ |
| 133 | CD8α | scFv | CD8α | CD28 | CD28 | OX40 | CD3ζ |
| 134 | CD8α | scFv | CD8α | CD28 | CD28 | OX40L | CD3ζ |
| 135 | CD8α | scFv | CD8α | CD28 | CD28 | ICOS | CD3ζ |
| 136 | CD8α | scFv | CD8α | CD28 | CD28 | DNAM-1 | CD3ζ |
| 137 | CD8α | scFv | CD8α | CD28 | CD28 | NKp30 | CD3ζ |
| 138 | CD8α | scFv | CD8α | CD28 | CD28 | NKp44 | CD3ζ |
| 139 | CD8α | scFv | CD8α | CD28 | CD28 | NKp46 | CD3ζ |
| 140 | CD8α | scFv | CD8α | CD28 | CD28 | NKG2D | CD3ζ |
| 141 | CD8α | scFv | CD8α | CD28 | CD28 | JAMAL | CD3ζ |
| 142 | CD8α | scFv | CD8α | CD28 | CD28 | 2B4 | CD3ζ |
| 143 | CD8α | scFv | CD8α | CD28 | 4-1BB | CD28 | CD3ζ |
| 144 | CD8α | scFv | CD8α | CD28 | 4-1BB | DAP10 | CD3ζ |
| 145 | CD8α | scFv | CD8α | CD28 | 4-1BB | DAP12 | CD3ζ |
| 146 | CD8α | scFv | CD8α | CD28 | 4-1BB | CD27 | CD3ζ |
| 147 | CD8α | scFv | CD8α | CD28 | 4-1BB | CD2 | CD3ζ |
| 148 | CD8α | scFv | CD8α | CD28 | 4-1BB | OX40 | CD3ζ |
| 149 | CD8α | scFv | CD8α | CD28 | 4-1BB | OX40L | CD3ζ |
| 150 | CD8α | scFv | CD8α | CD28 | 4-1BB | ICOS | CD3ζ |
| 151 | CD8α | scFv | CD8α | CD28 | 4-1BB | DNAM-1 | CD3ζ |
| 152 | CD8α | scFv | CD8α | CD28 | 4-1BB | NKp30 | CD3ζ |
| 153 | CD8α | scFv | CD8α | CD28 | 4-1BB | NKp44 | CD3ζ |
| 154 | CD8α | scFv | CD8α | CD28 | 4-1BB | NKp46 | CD3ζ |
| 155 | CD8α | scFv | CD8α | CD28 | 4-1BB | NKG2D | CD3ζ |
| 156 | CD8α | scFv | CD8α | CD28 | 4-1BB | JAMAL | CD3ζ |
| 157 | CD8α | scFv | CD8α | CD28 | 4-1BB | 2B4 | CD3ζ |
| 158 | CD8α | scFv | CD8α | CD28 | 2B4 | CD28 | CD3ζ |
| 159 | CD8α | scFv | CD8α | CD28 | 2B4 | DAP10 | CD3ζ |
| 160 | CD8α | scFv | CD8α | CD28 | 2B4 | DAP12 | CD3ζ |
| 161 | CD8α | scFv | CD8α | CD28 | 2B4 | CD27 | CD3ζ |
| 162 | CD8α | scFv | CD8α | CD28 | 2B4 | CD2 | CD3ζ |
| 163 | CD8α | scFv | CD8α | CD28 | 2B4 | OX40 | CD3ζ |
| 164 | CD8α | scFv | CD8α | CD28 | 2B4 | OX40L | CD3ζ |
| 165 | CD8α | scFv | CD8α | CD28 | 2B4 | ICOS | CD3ζ |
| 166 | CD8α | scFv | CD8α | CD28 | 2B4 | DNAM-1 | CD3ζ |
| 167 | CD8α | scFv | CD8α | CD28 | 2B4 | NKp30 | CD3ζ |
| 168 | CD8α | scFv | CD8α | CD28 | 2B4 | NKp44 | CD3ζ |
| 169 | CD8α | scFv | CD8α | CD28 | 2B4 | NKp46 | CD3ζ |
| 170 | CD8α | scFv | CD8α | CD28 | 2B4 | NKG2D | CD3ζ |
| 171 | CD8α | scFv | CD8α | CD28 | 2B4 | JAMAL | CD3ζ |
| 172 | CD8α | scFv | CD8α | CD28 | 2B4 | 2B4 | CD3ζ |
| 173 | CD8α | scFv | CD8α | 4-1BB | CD28 | 4-1BB | CD3ζ |
| 174 | CD8α | scFv | CD8α | 4-1BB | CD28 | DAP10 | CD3ζ |
| 175 | CD8α | scFv | CD8α | 4-1BB | CD28 | DAP12 | CD3ζ |
| 176 | CD8α | scFv | CD8α | 4-1BB | CD28 | CD27 | CD3ζ |
| 177 | CD8α | scFv | CD8α | 4-1BB | CD28 | CD2 | CD3ζ |
| 178 | CD8α | scFv | CD8α | 4-1BB | CD28 | OX40 | CD3ζ |
| 179 | CD8α | scFv | CD8α | 4-1BB | CD28 | OX40L | CD3ζ |
| 180 | CD8α | scFv | CD8α | 4-1BB | CD28 | ICOS | CD3ζ |
| 181 | CD8α | scFv | CD8α | 4-1BB | CD28 | DNAM-1 | CD3ζ |
| 182 | CD8α | scFv | CD8α | 4-1BB | CD28 | NKp30 | CD3ζ |
| 183 | CD8α | scFv | CD8α | 4-1BB | CD28 | NKp44 | CD3ζ |
| 184 | CD8α | scFv | CD8α | 4-1BB | CD28 | NKp46 | CD3ζ |
| 185 | CD8α | scFv | CD8α | 4-1BB | CD28 | NKG2D | CD3ζ |
| 186 | CD8α | scFv | CD8α | 4-1BB | CD28 | JAMAL | CD3ζ |
| 187 | CD8α | scFv | CD8α | 4-1BB | CD28 | 2B4 | CD3ζ |
| 188 | CD8α | scFv | CD8α | 4-1BB | 4-1BB | DAP10 | CD3ζ |
| 189 | CD8α | scFv | CD8α | 4-1BB | 4-1BB | DAP12 | CD3ζ |
| 190 | CD8α | scFv | CD8α | 4-1BB | 4-1BB | CD27 | CD3ζ |
| 191 | CD8α | scFv | CD8α | 4-1BB | 4-1BB | CD2 | CD3ζ |
| 192 | CD8α | scFv | CD8α | 4-1BB | 4-1BB | OX40 | CD3ζ |
| 193 | CD8α | scFv | CD8α | 4-1BB | 4-1BB | OX40L | CD3ζ |
| 194 | CD8α | scFv | CD8α | 4-1BB | 4-1BB | ICOS | CD3ζ |
| 195 | CD8α | scFv | CD8α | 4-1BB | 4-1BB | DNAM-1 | CD3ζ |
| 196 | CD8α | scFv | CD8α | 4-1BB | 4-1BB | NKp30 | CD3ζ |
| 197 | CD8α | scFv | CD8α | 4-1BB | 4-1BB | NKp44 | CD3ζ |
| 198 | CD8α | scFv | CD8α | 4-1BB | 4-1BB | NKp46 | CD3ζ |
| 199 | CD8α | scFv | CD8α | 4-1BB | 4-1BB | NKG2D | CD3ζ |
| 200 | CD8α | scFv | CD8α | 4-1BB | 4-1BB | JAMAL | CD3ζ |
| 201 | CD8α | scFv | CD8α | 4-1BB | 4-1BB | 2B4 | CD3ζ |
| 202 | CD8α | scFv | CD8α | 4-1BB | 2B4 | DAP10 | CD3ζ |
| 203 | CD8α | scFv | CD8α | 4-1BB | 2B4 | DAP12 | CD3ζ |
| 204 | CD8α | scFv | CD8α | 4-1BB | 2B4 | CD27 | CD3ζ |
| 205 | CD8α | scFv | CD8α | 4-1BB | 2B4 | CD2 | CD3ζ |
| 206 | CD8α | scFv | CD8α | 4-1BB | 2B4 | OX40 | CD3ζ |
| 207 | CD8α | scFv | CD8α | 4-1BB | 2B4 | OX40L | CD3ζ |
| 208 | CD8α | scFv | CD8α | 4-1BB | 2B4 | ICOS | CD3ζ |
| 209 | CD8α | scFv | CD8α | 4-1BB | 2B4 | DNAM-1 | CD3ζ |
| 210 | CD8α | scFv | CD8α | 4-1BB | 2B4 | NKp30 | CD3ζ |
| 211 | CD8α | scFv | CD8α | 4-1BB | 2B4 | NKp44 | CD3ζ |
| 212 | CD8α | scFv | CD8α | 4-1BB | 2B4 | NKp46 | CD3ζ |
| 213 | CD8α | scFv | CD8α | 4-1BB | 2B4 | NKG2D | CD3ζ |
| 214 | CD8α | scFv | CD8α | 4-1BB | 2B4 | JAMAL | CD3ζ |
| 215 | CD8α | scFv | CD8α | 4-1BB | 2B4 | 2B4 | CD3ζ |
| 216 | CD8α | scFv | CD8α | NKG2D | CD28 | CD28 | CD3ζ |
| 217 | CD8α | scFv | CD8α | NKG2D | CD28 | DAP10 | CD3ζ |
| 218 | CD8α | scFv | CD8α | NKG2D | CD28 | DAP12 | CD3ζ |
| 219 | CD8α | scFv | CD8α | NKG2D | CD28 | CD27 | CD3ζ |
| 220 | CD8α | scFv | CD8α | NKG2D | CD28 | CD2 | CD3ζ |
| 221 | CD8α | scFv | CD8α | NKG2D | CD28 | OX40 | CD3ζ |
| 222 | CD8α | scFv | CD8α | NKG2D | CD28 | OX40L | CD3ζ |
| 223 | CD8α | scFv | CD8α | NKG2D | CD28 | ICOS | CD3ζ |
| 224 | CD8α | scFv | CD8α | NKG2D | CD28 | DNAM-1 | CD3ζ |
| 225 | CD8α | scFv | CD8α | NKG2D | CD28 | NKp30 | CD3ζ |
| 226 | CD8α | scFv | CD8α | NKG2D | CD28 | NKp44 | CD3ζ |
| 227 | CD8α | scFv | CD8α | NKG2D | CD28 | NKp46 | CD3ζ |
| 228 | CD8α | scFv | CD8α | NKG2D | CD28 | NKG2D | CD3ζ |
| 229 | CD8α | scFv | CD8α | NKG2D | CD28 | JAMAL | CD3ζ |
| 230 | CD8α | scFv | CD8α | NKG2D | CD28 | 2B4 | CD3ζ |
| 231 | CD8α | scFv | CD8α | NKG2D | 4-1BB | DAP10 | CD3ζ |
| 232 | CD8α | scFv | CD8α | NKG2D | 4-1BB | DAP12 | CD3ζ |
| 233 | CD8α | scFv | CD8α | NKG2D | 4-1BB | CD27 | CD3ζ |
| 234 | CD8α | scFv | CD8α | NKG2D | 4-1BB | CD2 | CD3ζ |
| 235 | CD8α | scFv | CD8α | NKG2D | 4-1BB | OX40 | CD3ζ |
| 236 | CD8α | scFv | CD8α | NKG2D | 4-1BB | OX40L | CD3ζ |
| 237 | CD8α | scFv | CD8α | NKG2D | 4-1BB | ICOS | CD3ζ |
| 238 | CD8α | scFv | CD8α | NKG2D | 4-1BB | DNAM-1 | CD3ζ |
| 239 | CD8α | scFv | CD8α | NKG2D | 4-1BB | NKp30 | CD3ζ |
| 240 | CD8α | scFv | CD8α | NKG2D | 4-1BB | NKp44 | CD3ζ |
| 241 | CD8α | scFv | CD8α | NKG2D | 4-1BB | NKp46 | CD3ζ |
| 242 | CD8α | scFv | CD8α | NKG2D | 4-1BB | NKG2D | CD3ζ |
| 243 | CD8α | scFv | CD8α | NKG2D | 4-1BB | JAMAL | CD3ζ |
| 244 | CD8α | scFv | CD8α | NKG2D | 4-1BB | 2B4 | CD3ζ |
| 245 | CD8α | scFv | CD8α | NKG2D | 2B4 | CD28 | CD3ζ |
| 246 | CD8α | scFv | CD8α | NKG2D | 2B4 | DAP10 | CD3ζ |
| 247 | CD8α | scFv | CD8α | NKG2D | 2B4 | DAP12 | CD3ζ |
| 248 | CD8α | scFv | CD8α | NKG2D | 2B4 | CD27 | CD3ζ |
| 249 | CD8α | scFv | CD8α | NKG2D | 2B4 | CD2 | CD3ζ |
| 250 | CD8α | scFv | CD8α | NKG2D | 2B4 | OX40 | CD3ζ |
| 251 | CD8α | scFv | CD8α | NKG2D | 2B4 | OX40L | CD3ζ |
| 252 | CD8α | scFv | CD8α | NKG2D | 2B4 | ICOS | CD3ζ |
| 253 | CD8α | scFv | CD8α | NKG2D | 2B4 | DNAM-1 | CD3ζ |
| 254 | CD8α | scFv | CD8α | NKG2D | 2B4 | NKp30 | CD3ζ |
| 255 | CD8α | scFv | CD8α | NKG2D | 2B4 | NKp44 | CD3ζ |
| 256 | CD8α | scFv | CD8α | NKG2D | 2B4 | NKp46 | CD3ζ |
| 257 | CD8α | scFv | CD8α | NKG2D | 2B4 | NKG2D | CD3ζ |
| 258 | CD8α | scFv | CD8α | NKG2D | 2B4 | JAMAL | CD3ζ |

TABLE 13
Exemplary examples of CAR-γδ T cell constructs

| | | Antigen | Hinge | Transmem- | Co-stim. | Co-stim. | Cytoplasmic |
|---|---|---|---|---|---|---|---|
| | Signal | binding | domain | brane | domain | domain | Signaling |
| CAR # | Sequence | domain | (e) | domain (b) | (d1) | (d2) | domain (c) |
| 1 | CD8α | scFv | CD8α | CD8α | 4-1BB | none | CD3ζ |
| 2 | CD8α | scFv | CD8α | CD28 | 4-1BB | none | CD3ζ |
| 3 | CD8α | scFv | CD8α | 4-1BB | 4-1BB | none | CD3ζ |
| 4 | CD8α | scFv | CD8α | CD4 | 4-1BB | none | CD3ζ |
| 5 | CD8α | scFv | CD8α | FcεRIγ | 4-1BB | none | CD3ζ |
| 6 | CD8α | scFv | CD8α | CD8β | 4-1BB | none | CD3ζ |
| 7 | CD8α | scFv | CD8α | C16α | 4-1BB | none | CD3ζ |
| 8 | CD8α | scFv | CD8α | OX40 | 4-1BB | none | CD3ζ |
| 9 | CD8α | scFv | CD8α | CD3ζ | 4-1BB | none | CD3ζ |
| 10 | CD8α | scFv | CD8α | CD3ε | 4-1BB | none | CD3ζ |
| 11 | CD8α | scFv | CD8α | CD3γ | 4-1BB | none | CD3ζ |
| 12 | CD8α | scFv | CD8α | CD3δ | 4-1BB | none | CD3ζ |
| 13 | CD8α | scFv | CD8α | TCR-α | 4-1BB | none | CD3ζ |
| 14 | CD8α | scFv | CD8α | CD32 | 4-1BB | none | CD3ζ |
| 15 | CD8α | scFv | CD8α | CD64 | 4-1BB | none | CD3ζ |
| 16 | CD8α | scFv | CD8α | VEGFR2 | 4-1BB | none | CD3ζ |
| 17 | CD8α | scFv | CD8α | FAS | 4-1BB | none | CD3ζ |
| 18 | CD8α | scFv | CD8α | FGFR2B | 4-1BB | none | CD3ζ |
| 19 | CD8α | scFv | CD8α | CD8α | CD2 | none | CD3ζ |
| 20 | CD8α | scFv | CD8α | CD8α | CD28 | none | CD3ζ |
| 21 | CD8α | scFv | CD8α | CD8α | CD27 | none | CD3ζ |
| 22 | CD8α | scFv | CD8α | CD8α | ICOS | none | CD3ζ |
| 23 | CD8α | scFv | CD8α | CD8α | JAMAL | none | CD3ζ |
| 24 | CD8α | scFv | CD8α | CD8α | OX40 | none | CD3ζ |
| 25 | CD8α | scFv | CD8α | CD8α | OX40L | none | CD3ζ |
| 26 | CD8α | scFv | CD8α | CD8α | NKG2D | none | CD3ζ |
| 27 | CD8α | scFv | CD8α | CD8α | CD46 | none | CD3ζ |
| 28 | CD8α | scFv | CD8α | CD8α | DNAM-1 | none | CD3ζ |
| 29 | CD8α | scFv | CD8α | CD8α | NKp30 | none | CD3ζ |
| 30 | CD8α | scFv | CD8α | CD8α | NKp44 | none | CD3ζ |
| 31 | CD8α | scFv | CD8α | CD8α | DAP10 | none | CD3ζ |
| 32 | CD8α | scFv | CD8α | CD28 | CD2 | none | CD3ζ |
| 33 | CD8α | scFv | CD8α | CD28 | 4-1BB | none | CD3ζ |
| 34 | CD8α | scFv | CD8α | CD28 | CD28 | none | CD3ζ |
| 35 | CD8α | scFv | CD8α | CD28 | CD27 | none | CD3ζ |
| 36 | CD8α | scFv | CD8α | CD28 | ICOS | none | CD3ζ |
| 37 | CD8α | scFv | CD8α | CD28 | JAMAL | none | CD3ζ |
| 38 | CD8α | scFv | CD8α | CD28 | OX40 | none | CD3ζ |
| 39 | CD8α | scFv | CD8α | CD28 | OX40L | none | CD3ζ |
| 40 | CD8α | scFv | CD8α | CD28 | NKG2D | none | CD3ζ |
| 41 | CD8α | scFv | CD8α | CD28 | CD46 | none | CD3ζ |
| 42 | CD8α | scFv | CD8α | CD28 | DNAM-1 | none | CD3ζ |
| 43 | CD8α | scFv | CD8α | CD28 | NKp30 | none | CD3ζ |
| 44 | CD8α | scFv | CD8α | CD28 | NKp44 | none | CD3ζ |
| 45 | CD8α | scFv | CD8α | CD28 | DAP10 | none | CD3ζ |
| 46 | CD8α | scFv | CD8α | 4-1BB | CD2 | none | CD3ζ |
| 47 | CD8α | scFv | CD8α | 4-1BB | 4-1BB | none | CD3ζ |
| 48 | CD8α | scFv | CD8α | 4-1BB | CD28 | none | CD3ζ |
| 49 | CD8α | scFv | CD8α | 4-1BB | CD27 | none | CD3ζ |
| 50 | CD8α | scFv | CD8α | 4-1BB | ICOS | none | CD3ζ |
| 51 | CD8α | scFv | CD8α | 4-1BB | JAMAL | none | CD3ζ |
| 52 | CD8α | scFv | CD8α | 4-1BB | OX40 | none | CD3ζ |
| 53 | CD8α | scFv | CD8α | 4-1BB | OX40L | none | CD3ζ |
| 54 | CD8α | scFv | CD8α | 4-1BB | NKG2D | none | CD3ζ |
| 55 | CD8α | scFv | CD8α | 4-1BB | CD46 | none | CD3ζ |
| 56 | CD8α | scFv | CD8α | 4-1BB | DNAM-1 | none | CD3ζ |
| 57 | CD8α | scFv | CD8α | 4-1BB | NKp30 | none | CD3ζ |
| 58 | CD8α | scFv | CD8α | 4-1BB | NKp44 | none | CD3ζ |
| 59 | CD8α | scFv | CD8α | 4-1BB | DAP10 | none | CD3ζ |
| 60 | CD8α | scFv | CD8α | CD8α | CD28 | 4-1BB | CD3ζ |
| 61 | CD8α | scFv | CD8α | CD8α | CD28 | CD2 | CD3ζ |
| 62 | CD8α | scFv | CD8α | CD8α | CD28 | CD27 | CD3ζ |
| 63 | CD8α | scFv | CD8α | CD8α | CD28 | ICOS | CD3ζ |
| 64 | CD8α | scFv | CD8α | CD8α | CD28 | JAMAL | CD3ζ |
| 65 | CD8α | scFv | CD8α | CD8α | CD28 | OX40 | CD3ζ |
| 66 | CD8α | scFv | CD8α | CD8α | CD28 | OX40L | CD3ζ |
| 67 | CD8α | scFv | CD8α | CD8α | CD28 | NKG2D | CD3ζ |
| 68 | CD8α | scFv | CD8α | CD8α | CD28 | CD46 | CD3ζ |
| 69 | CD8α | scFv | CD8α | CD8α | CD28 | DNAM-1 | CD3ζ |
| 70 | CD8α | scFv | CD8α | CD8α | CD28 | NKp30 | CD3ζ |
| 71 | CD8α | scFv | CD8α | CD8α | CD28 | NKp44 | CD3ζ |
| 72 | CD8α | scFv | CD8α | CD8α | CD28 | DAP10 | CD3ζ |
| 73 | CD8α | scFv | CD8α | CD8α | 4-1BB | CD2 | CD3ζ |
| 74 | CD8α | scFv | CD8α | CD8α | 4-1BB | CD27 | CD3ζ |
| 75 | CD8α | scFv | CD8α | CD8α | 4-1BB | ICOS | CD3ζ |
| 76 | CD8α | scFv | CD8α | CD8α | 4-1BB | JAMAL | CD3ζ |
| 77 | CD8α | scFv | CD8α | CD8α | 4-1BB | OX40 | CD3ζ |
| 78 | CD8α | scFv | CD8α | CD8α | 4-1BB | OX40L | CD3ζ |
| 79 | CD8α | scFv | CD8α | CD8α | 4-1BB | NKG2D | CD3ζ |
| 80 | CD8α | scFv | CD8α | CD8α | 4-1BB | CD46 | CD3ζ |
| 81 | CD8α | scFv | CD8α | CD8α | 4-1BB | DNAM-1 | CD3ζ |
| 82 | CD8α | scFv | CD8α | CD8α | 4-1BB | NKp30 | CD3ζ |
| 83 | CD8α | scFv | CD8α | CD8α | 4-1BB | NKp44 | CD3ζ |
| 84 | CD8α | scFv | CD8α | CD8α | 4-1BB | DAP10 | CD3ζ |
| 85 | CD8α | scFv | CD8α | CD28 | CD28 | 4-1BB | CD3ζ |
| 86 | CD8α | scFv | CD8α | CD28 | CD28 | CD2 | CD3ζ |
| 87 | CD8α | scFv | CD8α | CD28 | CD28 | CD27 | CD3ζ |
| 88 | CD8α | scFv | CD8α | CD28 | CD28 | ICOS | CD3ζ |
| 89 | CD8α | scFv | CD8α | CD28 | CD28 | JAMAL | CD3ζ |
| 90 | CD8α | scFv | CD8α | CD28 | CD28 | OX40 | CD3ζ |
| 91 | CD8α | scFv | CD8α | CD28 | CD28 | OX40L | CD3ζ |
| 92 | CD8α | scFv | CD8α | CD28 | CD28 | NKG2D | CD3ζ |
| 93 | CD8α | scFv | CD8α | CD28 | CD28 | CD46 | CD3ζ |
| 94 | CD8α | scFv | CD8α | CD28 | CD28 | DNAM-1 | CD3ζ |
| 95 | CD8α | scFv | CD8α | CD28 | CD28 | NKp30 | CD3ζ |
| 96 | CD8α | scFv | CD8α | CD28 | CD28 | NKp44 | CD3ζ |
| 97 | CD8α | scFv | CD8α | CD28 | CD28 | DAP10 | CD3ζ |
| 98 | CD8α | scFv | CD8α | CD28 | 4-1BB | CD2 | CD3ζ |
| 99 | CD8α | scFv | CD8α | CD28 | 4-1BB | CD27 | CD3ζ |
| 100 | CD8α | scFv | CD8α | CD28 | 4-1BB | ICOS | CD3ζ |
| 101 | CD8α | scFv | CD8α | CD28 | 4-1BB | JAMAL | CD3ζ |
| 102 | CD8α | scFv | CD8α | CD28 | 4-1BB | OX40 | CD3ζ |
| 103 | CD8α | scFv | CD8α | CD28 | 4-1BB | OX40L | CD3ζ |
| 104 | CD8α | scFv | CD8α | CD28 | 4-1BB | NKG2D | CD3ζ |
| 105 | CD8α | scFv | CD8α | CD28 | 4-1BB | CD46 | CD3ζ |
| 106 | CD8α | scFv | CD8α | CD28 | 4-1BB | DNAM-1 | CD3ζ |
| 107 | CD8α | scFv | CD8α | CD28 | 4-1BB | NKp30 | CD3ζ |
| 108 | CD8α | scFv | CD8α | CD28 | 4-1BB | NKp44 | CD3ζ |
| 109 | CD8α | scFv | CD8α | CD28 | 4-1BB | DAP10 | CD3ζ |
| 110 | CD8α | scFv | CD8α | 4-1BB | CD28 | CD2 | CD3ζ |
| 111 | CD8α | scFv | CD8α | 4-1BB | CD28 | CD27 | CD3ζ |
| 112 | CD8α | scFv | CD8α | 4-1BB | CD28 | ICOS | CD3ζ |
| 113 | CD8α | scFv | CD8α | 4-1BB | CD28 | JAMAL | CD3ζ |
| 114 | CD8α | scFv | CD8α | 4-1BB | CD28 | OX40 | CD3ζ |
| 115 | CD8α | scFv | CD8α | 4-1BB | CD28 | OX40L | CD3ζ |
| 116 | CD8α | scFv | CD8α | 4-1BB | CD28 | NKG2D | CD3ζ |
| 117 | CD8α | scFv | CD8α | 4-1BB | CD28 | CD46 | CD3ζ |
| 118 | CD8α | scFv | CD8α | 4-1BB | CD28 | DNAM-1 | CD3ζ |
| 119 | CD8α | scFv | CD8α | 4-1BB | CD28 | NKp30 | CD3ζ |
| 120 | CD8α | scFv | CD8α | 4-1BB | CD28 | NKp44 | CD3ζ |
| 121 | CD8α | scFv | CD8α | 4-1BB | CD28 | DAP10 | CD3ζ |
| 122 | CD8α | scFv | CD8α | 4-1BB | 4-1BB | CD2 | CD3ζ |
| 123 | CD8α | scFv | CD8α | 4-1BB | 4-1BB | CD27 | CD3ζ |
| 124 | CD8α | scFv | CD8α | 4-1BB | 4-1BB | ICOS | CD3ζ |
| 125 | CD8α | scFv | CD8α | 4-1BB | 4-1BB | JAMAL | CD3ζ |
| 126 | CD8α | scFv | CD8α | 4-1BB | 4-1BB | OX40 | CD3ζ |
| 127 | CD8α | scFv | CD8α | 4-1BB | 4-1BB | OX40L | CD3ζ |
| 128 | CD8α | scFv | CD8α | 4-1BB | 4-1BB | NKG2D | CD3ζ |
| 129 | CD8α | scFv | CD8α | 4-1BB | 4-1BB | CD46 | CD3ζ |
| 130 | CD8α | scFv | CD8α | 4-1BB | 4-1BB | DNAM-1 | CD3ζ |
| 131 | CD8α | scFv | CD8α | 4-1BB | 4-1BB | NKp30 | CD3ζ |
| 132 | CD8α | scFv | CD8α | 4-1BB | 4-1BB | NKp44 | CD3ζ |
| 133 | CD8α | scFv | CD8α | 4-1BB | 4-1BB | DAP10 | CD3ζ |

TABLE 14
Exemplary examples of GPC3 targeting CAR-T cell constructs

| | Extracellular | | | Co- | Cyto- |
|---|---|---|---|---|---|
| | domain | | Trans- | stim- | plasmic |
| Signal | (antigen | Hinge | membrane | ulatory | Signaling |
| Sequence | binding) | domain | domain | domain | domain |
| CD8α | scFv (e.g., | CD8α | CD8 | 4-1BB | CD3ζ |
| | anti-GPC3 scFv) | | | | |
| CD8α | scFv (e.g., | CD28 | CD28 | CD28 | CD3ζ |
| | anti-GPC3 scFv) | | | | |

Amino acid sequences of an exemplary anti-GPC3 scFv for constructing anti-GPC3 CAR constructs is SEQ ID NO: 85, exemplary anti-GPC3 CAR constructs comprising such scFv are provided by SEQ ID NO: 86 and SEQ ID NO: 87.

Anti-GPC3 scFv derived from GC33 (SEQ ID NO: 85):
DVVMTQSPLSLPVTPGEPASISCRSSQSLVHSNRNTYLHWYLQKPGQSPQLLIYKVS
NRFSGVPDRFSGSGSGTDFTLKISRVEAEDVGVYYCSQNTHVPPTFGQGTKLEIKRG
GGGSGGGGSGGGGSQVQLVQSGAEVKKPGASVKVSCKASGYTFTDYEMHWVRQAPGQ
GLEWMGALDPKTGDTAYSQKFKGRVTLTADKSTSTAYMELSSLTSEDTAVYYCTRFY
SYTYWGQGTLVTVSS
Anti-GPC3-CAR 1 (4-1BB co-stimulatory domain/CD3ζ cytoplasmic domain)
(SEQ ID NO: 86):
MALPVTALLLPLALLLHAARPDVVMTQSPLSLPVTPGEPASISCRSSQSLVHSNRNT
YLHWYLQKPGQSPQLLIYKVSNRFSGVPDRESGSGSGTDFTLKISRVEAEDVGVYYC
SQNTHVPPTFGQGTKLEIKRGGGGSGGGGSGGGGSQVQLVQSGAEVKKPGASVKVSC
KASGYTFTDYEMHWVRQAPGQGLEWMGALDPKTGDTAYSQKFKGRVTLTADKSTSTA
YMELSSLTSEDTAVYYCTRFYSYTYWGQGTLVTVSSTTTPAPRPPTPAPTIASQPLS
LRPEACRPAAGGAVHTRGLDFACDIYIWAPLAGTCGVLLLSLVITLYCKRGRKKLLY
IFKQPFMRPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEIGMKGERR
RGKGHDGLYQGLSTATKDTYDALHMQALPPR
Anti-GPC3-CAR 2 (CD28 transmembrane domain (bold)/CD28 co-stimulatory
domain/CD3ζ cytoplasmic domain (SEQ ID NO: 87)
MALPVTALLLPLALLLHAARPDVVMTQSPLSLPVTPGEPASISCRSSQSLVHSNRNT
YLHWYLQKPGQSPQLLIYKVSNRFSGVPDRESGSGSGTDFTLKISRVEAEDVGVYYC
SQNTHVPPTFGQGTKLEIKRGGGGSGGGGSGGGGSQVQLVQSGAEVKKPGASVKVSC
KASGYTFTDYEMHWVRQAPGQGLEWMGALDPKTGDTAYSQKFKGRVTLTADKSTSTA
YMELSSLTSEDTAVYYCTRFYSYTYWGQGTLVTVSSIEVMYPPPYLDNEKSNGTIIH
VKGKHLCPSPLFPGPSKPFWVLVVVGGVLACYSLLVTVAFIIFWVRSKRSRLLHSDY
MNMTPRRPGPTRKHYQPYAPPRDFAAYRSRVKFSRSADAPAYQQGQNQLYNELNLGR
REEYDVLDKRRGRDPEMGGKPRRKNPQEGLYNELQKDKMAEAYSEIGMKGERRRGKG
HDGLYQGLSTATKDTYDALHMQALPPR

An exemplary anti-GPC3 CAR construct comprising such scFv suitable for coexpression of metabolism modifying polypeptides GOT2 and TIGAR is provided by SEQ ID NO: 92 (transient translation product prior to cleavage at ribosomal skipping sites P2A and T2A):

transient translation product of anti-GPC3 CAR-P2A-GOT2-T2A-TIGAR (4-1BB co-
stimulatory domain/CD3ζ cytoplasmic domain/GSG linker/P2A/GOT2/GSG
linker T2A/TIGAR) (SEQ ID NO: 92):
MALPVTALLLPLALLLHAARPDVVMTQSPLSLPVTPGEPASISCRSSQSLVHSNRNT
YLHWYLQKPGQSPQLLIYKVSNRFSGVPDRESGSGSGTDFTLKISRVEAEDVGVYYC
SQNTHVPPTFGQGTKLEIKRGGGGSGGGGSGGGGSQVQLVQSGAEVKKPGASVKVSC
KASGYTFTDYEMHWVRQAPGQGLEWMGALDPKTGDTAYSQKFKGRVTLTADKSTSTA
YMELSSLTSEDTAVYYCTRFYSYTYWGQGTLVTVSSTTTPAPRPPTPAPTIASQPLS
LRPEACRPAAGGAVHTRGLDFACDIYIWAPLAGTCGVLLLSLVITLYCKRGRKKLLY
IFKQPFMRPVQTTQEEDGCSCRFPEEEEGGCELRVKESRSADAPAYQQGQNQLYNEL
NLGRREEYDVLDKRRGRDPEMGGKPRRKNPQEGLYNELQKDKMAEAYSEIGMKGERR
RGKGHDGLYQGLSTATKDTYDALHMQALPPRGSGATNFSLLKQAGDVEENPGPMALL
HSGRVLPGIAAAFHPGLAAAASARASSWWTHVEMGPPDPILGVTEAFKRDINSKKMN
LGVGAYRDDNGKPYVLPSVRKAEAQIAAKNLDKEYLPIGGLAEFCKASAELALGENS
EVLKSGRFVTVQTISGTGALRIGASFLQRFFKFSRDVFLPKPTWGNHTPIFRDAGMQ
LQGYRYYDPKTCGFDFTGAVEDISKIPEQSVLLLHACAHNPTGVDPRPEQWKEIATV
VKKRNLFAFFDMAYQGFASGDGDKDAWAVRHFIEQGINVCLCQSYAKNMGLYGERVG
AFTMVCKDADEAKRVESQLKILIRPMYSNPPLNGARIAAAILNTPDLRKQWLQEVKV
MADRIIGMRTQLVSNLKKEGSTHNWQHITDQIGMFCFTGLKPEQVERLIKEFSIYMT
KDGRISVAGVISSNVGYLAHAIHQVTKGSGEGRGSLLTCGDVEENPGPMARFALTVV
RHGETRFNKEKIIQGQGVDEPLSETGFKQAAAAGIFLNNVKFTHAFSSDLMRTKQTM
HGILERSKFCKDMTVKYDSRLRERKYGVVEGKALSELRAMAKAAREECPVFTPPGGE
TLDQVKMRGIDFFEFLCQLILKEADQKEQFSQGSPSNCLETSLAEIFPLGKNHSSKV
NSDSGIPGLAASVLVVSHGAYMRSLFDYFLIDLKCSLPATLSRSELMSVTPNIGMSL
FIINFEEGREVKPTVQCICMNLQDHLNGLTETR

Amino acid sequences of an exemplary anti-ROR1 scFv for constructing anti-ROR1 CAR constructs is SEQ ID NO: 93, exemplary anti-ROR1 CAR constructs comprising such scFv is provided by SEQ ID NO 94.

Anti-ROR1 scFv (SEQ ID NO 93):
DIVMTQSPLSQPVTPGEPASISCRSSQSLLHRYGYNSLHWYLQKPGQSPQLLIYLGS
NRASGVPDRFSGSGSGTDFTLKVSRVEAEDVGVYYCMQALQTPYTFGQGTKLEIKGS
TSGSGKPGSGEGSTKGQVQLQESGPGLVKPSGTLSLTCAVSGGSISSSNWWSWVRQP
PGKGLEWLGEISHSGITNYNPSLKSRVTISVDKSKNHFSLKLNSVTAADTAVYYCTK
KWELLAFDFWGQGTMVTVSS
anti-ROR1 CAR (1730): anti-ROR scFv with CD8a signal sequence (italic)/
anti-ROR1 scFv/IgG4 hinge/ CD28 transmembrane domain/4-1BB Co-stimulatory
domain/CD3ζ cytoplasmic tail (SEQ ID NO: 94):
MALPVTALLLPLALLLHAARPDIVMTQSPLSQPVTPGEPASISCRSSQSLLHRYGYN
SLHWYLQKPGQSPQLLIYLGSNRASGVPDRFSGSGSGTDFTLKVSRVEAEDVGVYYC
MQALQTPYTFGQGTKLEIKGSTSGSGKPGSGEGSTKGQVQLQESGPGLVKPSGTLSL
TCAVSGGSISSSNWWSWVRQPPGKGLEWLGEISHSGITNYNPSLKSRVTISVDKSKN
HFSLKLNSVTAADTAVYYCIKKWELLAFDFWGQGTMVTVSSESKYGPPCPPCPFWVL
VVVGGVLACYSLLVTVAFIIFWVKRGRKKLLYIFKQPFMRPVQTTQEEDGCSCREPE
EEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHM
QALPPR

Exemplary anti-ROR1 CAR constructs comprising such scFv suitable for coexpression of metabolism modifying polypeptides GOT2 and/or TIGAR are provided by SEQ ID NOs: 95 to 97 (transient translation products prior to cleavage at ribosomal skipping sites P2A and T2A):

Transient translation product of anti-ROR1 CAR co-expressing TIGAR
(1767): 1730 CAR/GSG linker/P2A/TIGAR (SEQ ID NO: 95)
MALPVTALLLPLALLLHAARPDIVMTQSPLSQPVTPGEPASISCRSSQSLLHRYGYN
SLHWYLQKPGQSPQLLIYLGSNRASGVPDRFSGSGSGTDFTLKVSRVEAEDVGVYYC
MQALQTPYTFGQGTKLEIKGSTSGSGKPGSGEGSTKGQVQLQESGPGLVKPSGTLSL
TCAVSGGSISSSNWWSWVRQPPGKGLEWLGEISHSGITNYNPSLKSRVTISVDKSKN
HFSLKLNSVTAADTAVYYCTKKWELLAFDFWGQGIMVTVSSESKYGPPCPPCPFWVL
VVVGGVLACYSLLVTVAFIIFWVKRGRKKLLYIFKQPFMRPVQTTQEEDGCSCREPE
EEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHM
QALPPRGSGATNFSLLKQAGDVEENPGPMARFALTVVRHGETRENKEKIIQGQGVDE
PLSETGFKQAAAAGIFLNNVKFTHAFSSDLMRTKQTMHGILERSKFCKDMTVKYDSR
LRERKYGVVEGKALSELRAMAKAAREECPVFTPPGGETLDQVKMRGIDFFEFLCQLI
LKEADQKEQFSQGSPSNCLETSLAEIFPLGKNHSSKVNSDSGIPGLAASVLVVSHGA
YMRSLFDYFLTDLKCSLPATLSRSELMSVTPNTGMSLFIINFEEGREVKPTVQCICM
NLQDHLNGLTETR
Transient translation product of anti-ROR1 CAR co-expressing GOT2
(1768): 1730 CAR/GSG linker/P2A/GOT2 (SEQ ID NO: 96):
MALPVTALLLPLALLLHAARPDIVMTQSPLSQPVTPGEPASISCRSSQSLLHRYGYN
SLHWYLQKPGQSPQLLIYLGSNRASGVPDRFSGSGSGTDFTLKVSRVEAEDVGVYYC
MQALQTPYTFGQGTKLEIKGSTSGSGKPGSGEGSTKGQVQLQESGPGLVKPSGTLSL
TCAVSGGSISSSNWWSWVRQPPGKGLEWLGEISHSGITNYNPSLKSRVTISVDKSKN
HFSLKLNSVTAADTAVYYCTKKWELLAFDFWGQGIMVTVSSESKYGPPCPPCPFWVL
VVVGGVLACYSLLVTVAFIIFWVKRGRKKLLYIFKQPFMRPVQTTQEEDGCSCREPE
EEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHM
QALPPRGSGATNFSLLKQAGDVEENPGPMALLHSGRVLPGIAAAFHPGLAAAASARA
SSWWTHVEMGPPDPILGVTEAFKRDTNSKKMNLGVGAYRDDNGKPYVLPSVRKAEAQ
IAAKNLDKEYLPIGGLAEFCKASAELALGENSEVLKSGRFVTVQTISGTGALRIGAS
FLQRFFKFSRDVFLPKPTWGNHTPIFRDAGMQLQGYRYYDPKTCGFDFTGAVEDISK
IPEQSVLLLHACAHNPTGVDPRPEQWKEIATVVKKRNLFAFFDMAYQGFASGDGDKD
AWAVRHFIEQGINVCLCQSYAKNMGLYGERVGAFTMVCKDADEAKRVESQLKILIRP
MYSNPPLNGARIAAAILNTPDLRKQWLQEVKVMADRIIGMRTQLVSNLKKEGSTHNW
QHITDQIGMFCFTGLKPEQVERLIKEFSIYMTKDGRISVAGVTSSNVGYLAHAIHQV
TK
Transient translation product of anti-ROR1 CAR co-expressing GOT2 and
TIGAR (1798): 1730 CAR/GSG linker/P2A/GOT2/GSG linker/T2A/TIGAR (SEQ ID
NO: 97):
MALPVTALLLPLALLLHAARPDIVMTQSPLSQPVTPGEPASISCRSSQSLLHRYGYN
SLHWYLQKPGQSPQLLIYLGSNRASGVPDRESGSGSGTDFTLKVSRVEAEDVGVYYC
MQALQTPYTFGQGTKLEIKGSTSGSGKPGSGEGSTKGQVQLQESGPGLVKPSGTLSL
TCAVSGGSISSSNWWSWVRQPPGKGLEWLGEISHSGITNYNPSLKSRVTISVDKSKN
HFSLKLNSVTAADTAVYYCTKKWELLAFDFWGQGIMVTVSSESKYGPPCPPCPFWVL
VVVGGVLACYSLLVTVAFIIFWVKRGRKKLLYIFKQPFMRPVQTTQEEDGCSCRFPE
EEEGGCELRVKESRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHM
QALPPRGSGATNFSLLKQAGDVEENPGPMALLHSGRVLPGIAAAFHPGLAAAASARA
SSWWTHVEMGPPDPILGVTEAFKRDTNSKKMNLGVGAYRDDNGKPYVLPSVRKAEAQ
IAAKNLDKEYLPIGGLAEFCKASAELALGENSEVLKSGRFVTVQTISGTGALRIGAS
FLQRFFKFSRDVFLPKPTWGNHTPIFRDAGMQLQGYRYYDPKTCGFDETGAVEDISK
IPEQSVLLLHACAHNPTGVDPRPEQWKEIATVVKKRNLFAFFDMAYQGFASGDGDKD
AWAVRHFIEQGINVCLCQSYAKNMGLYGERVGAFTMVCKDADEAKRVESQLKILIRP
MYSNPPLNGARIAAAILNTPDLRKQWLQEVKVMADRIIGMRTQLVSNLKKEGSTHNW
QHITDQIGMFCFTGLKPEQVERLIKEFSIYMTKDGRISVAGVISSNVGYLAHAIHQV
TKGSGEGRGSLLTCGDVEENPGPMARFALTVVRHGETRENKEKIIQGQGVDEPLSET
GFKQAAAAGIFLNNVKFTHAFSSDLMRTKQTMHGILERSKFCKDMTVKYDSRLRERK
YGVVEGKALSELRAMAKAAREECPVFTPPGGETLDQVKMRGIDFFEFLCQLILKEAD
QKEQFSQGSPSNCLETSLAEIFPLGKNHSSKVNSDSGIPGLAASVLVVSHGAYMRSL
FDYFLTDLKCSLPATLSRSELMSVTPNTGMSLFIINFEEGREVKPTVQCICMNLQDH
LNGLTETR

### III. Immune Cells Expressing Polypeptides Modulating Metabolism and Optionally Chimeric Receptor Polypeptides

Provided herein are genetically engineered immune cells (e.g., T cells or NK cells) that co-express at least two metabolism modulating polypeptides as described herein.

Both innate and adaptive immune cells play an important role in cancer, autoimmune and pathogenic diseases. Non-limiting examples of immune cells include T cells (CD8⁺ T cells, effector CD4⁺ T cells e.g., αβ T cells, γδ T cells, Regulatory T Cells (Treg)), B cells, Natural Killer (NK) cells, Natural Killer T cells (NKT cells), Dendritic cells (DCs), Macrophages (e.g., M1, M2), Neutrophils (e.g., N1, N2), Eosinophils, Mast cells and Myeloid-derived suppressor cells (MDSCs). With respect to tumor treatment and treatment of infectious diseases, suitable immune cells are selected from activating immune cell types including B cells, T cells (e.g., CD8⁺ T cells, effector CD4⁺ T cells), NK cells, NKT cells, DCs, Macrophages (e.g., M1) and Neutrophils (e.g., N1). With respect to autoimmune diseases, or immune suppressive conditions (e.g., TME) suitable immune cells are selected from inhibitory immune cells including Tregs, Macrophages (e.g., M2), Neutrophils (e.g., N2), MDSCs (e.g., polymorphonuclear MDSCs (PMN-MDSCs) and Monocytic MDSCs (M-MDSCs) (Galli et al., J Exp Clin Cancer Res, 39(1): 89 (2020); Dong et al., Front Immunol, 12:609762 (2021)).

In some embodiments, these metabolism modulating polypeptides of the present invention are encoded by transgenes introduced into the immune cells (e.g., exogenous to the immune cells). The genetically engineered immune cells further express a chimeric receptor polypeptide (e.g., ACTR-T cells, or CAR-T cells) as also described herein. In some embodiments, the genetically engineered immune cells can be natural killer (NK) cells, monocytes/macrophages, neutrophils, eosinophils, αβ T or γδ T cells. For example, the T cells can be CD4⁺ helper cells or CD8⁺ cytotoxic cells, or a combination thereof. Alternatively, or in addition, the T cells can be suppressive T cells such as T_{reg} cells. In a preferred embodiment, the immune cell is an αβ T cell, and wherein the chimeric receptor polypeptide is a CAR polypeptide that comprises components as shown in Table 11. In another preferred embodiment, the immune cell is a NK cell, and wherein the chimeric receptor polypeptide is a CAR polypeptide that comprises components as shown in Table 12. In yet another preferred embodiment the immune cell is a γδ T cell, and wherein the chimeric receptor polypeptide is a CAR polypeptide that comprises components as shown in Table 13.

In some other embodiments, the genetically engineered immune cells described herein can be derived from a cell line, e.g., selected from NK-92, NK-92 MI, YTS, and KHYG-1, preferably NK-92 cells. In other embodiments, the genetically engineered immune cells described herein can be derived from peripheral blood mononuclear cells (PBMC), hematopoietic stem cells (HSCs), cord blood stem cells (CBSCs) or induced pluripotent stem cells (iPSCs). In other embodiments, the immune cell population described herein can be obtained from other sources, such as bone marrow, or tissues such as spleen, lymph node, thymus, or tumor tissue. In some embodiments, the starting population of NK or T cells is obtained from isolating mononuclear cells using ficoll-paque density gradient. In some embodiments, the method further comprises depleting the mononuclear cells of CD3, CD14, and/or CD19 cells to obtain the starting population of NK cells. In some embodiments, the method further comprises depleting the mononuclear cells CD3, CD14, and CD19 cells to obtain the starting population of NK cells. In a particular instance, depleting comprises performing magnetic sorting. In some embodiments, NK cells could be positively selected using sorting, magnetic bead selection or other methods to obtain the starting populations of NK cells.

In one embodiment, specifically lymphocytes are obtained from tumor tissue, i.e., tumor infiltrating lymphocytes (TILs). A source suitable for obtaining the type of immune cells desired would be evident to one of skill in the art. In some examples, the immune cells can be a mixture of different types of T cells and/or NK cells as known in the art. For example, the immune cells can be a population of immune cells isolated from a suitable donor (e.g., a human patient). In a preferred embodiment, the population of immune cells is derived from PBMCs, which may be obtained from the patient (e.g., a human patient) who is in need for the treatment described herein and who will be treated with the genetically engineered immune cells described herein (autologous approach). The type of immune cells desired (e.g., T cells or NK cells) may be expanded within the population of cells obtained by co-incubating the cells with stimulatory molecules. As a non-limiting example, anti-CD3 and anti-CD28 antibodies as well as cytokines such as IL-2 may be used for expansion of T cells.

Additionally, immune cells such as NK cells are derived from cord blood stem cells or induced pluripotent stem cells (iPSCs) providing from “off-the shelf” source for immunotherapy (Li et al., Cell Stem Cell, 23(2): 181-192.e185 (2018); Liu et al., Leukemia, 32(2): 520-531 (2018); Morgan et al., Front Immunol, 11:1965 (2020); Wrona, Borowiec et al., Int J Mol Sci, 22(11): (2021)). In particular embodiments, the starting population of NK cells is obtained from cord blood. In other embodiments, the cord blood has previously been frozen. In some embodiments, cells are derived from cell lines (e.g., NK-92 and Vγ9Vδ2 T cell).

In some embodiments, the genetically engineered immune cells (e.g., T cells or NK cells) may co-express any of the CAR constructs with at least two metabolism modulating polypeptides such as those disclosed herein. In some embodiments, the CAR construct may comprise a co-stimulatory domain from 4-1BB or CD28 and the metabolism modulating polypeptides. The CAR construct may further comprise a hinge and transmembrane domain from CD8 (e.g., CD8α) or CD28.

In some examples, the genetically engineered immune cells (e.g., T cells or NK cells) may be engineered to co-express any of the CAR constructs (e.g., the anti-GPC3 CAR or ROR1 CAR disclosed herein) and the transgene(s) encoding the metabolism modulating polypeptides (e.g., GOT2 and TIGAR). In specific embodiments, the genetically engineered immune cells comprise T or NK cells co-expressing the CAR and the transgenes GOT2 and TIGAR. In some embodiments, the genetically engineered immune cells comprise T or NK cells co-expressing the CAR and the transgenes GOT2 and GLUT1. In some embodiments, the genetically engineered immune cells comprise T or NK cells co-expressing the CAR and the transgenes GOT2 and PDK1. In some embodiments, the genetically engineered immune cells comprise T or NK cells co-expressing the CAR and the transgenes TIGAR and GLUT1. In some embodiments, the genetically engineered immune cells comprise T or NK cells coexpressing the CAR and the transgenes PDK1 and CTH. In some embodiments, the genetically engineered immune cells comprise T or NK cells co-expressing the CAR and the transgenes CTH and PSPH. In some embodiments, the genetically engineered immune cells comprise T or NK cells co-expressing the CAR and the transgenes GLUT1 and ASS1. In some embodiments, the genetically engineered immune cells comprise T or NK cells coexpressing the CAR and the transgenes GLUT1 and PSPH.

In other embodiments, the genetically engineered immune cells (e.g., T cells or NK cells) may co-express any of the ACTR constructs with at least two metabolism modulating polypeptides such as those disclosed herein. In some embodiments, the ACTR construct may comprise a co-stimulatory domain from 4-1BB or CD28. The ACTR constructs may further comprise a hinge and transmembrane domain from CD8 or CD28.

In some examples, the genetically engineered immune cells (e.g., T cells or NK cells) may be engineered to co-express any of the ACTR constructs (e.g., the CD16A-V158 ACTR disclosed herein) and the transgene(s) encoding the metabolism modulating polypeptides (e.g., GOT2 and TIGAR). In specific embodiments, the genetically engineered immune cells comprise T or NK cells co-expressing the ACTR and the transgenes GOT2 and TIGAR. In some embodiments, the genetically engineered immune cells comprise T or NK cells coexpressing the ACTR and the transgenes GOT2 and GLUT1. In some embodiments, the genetically engineered immune cells comprise T or NK cells co-expressing the ACTR and the transgenes GOT2 and PDK1. In some embodiments, the genetically engineered immune cells comprise T or NK cells co-expressing the ACTR and the transgenes TIGAR and GLUT1. In some embodiments, the genetically engineered immune cells comprise T or NK cells co-expressing the ACTR and the transgenes PDK1 and CTH. In some embodiments, the genetically engineered immune cells comprise T or NK cells co-expressing the ACTR and the transgenes CTH and PSPH. In some embodiments, the genetically engineered immune cells comprise T or NK cells co-expressing the ACTR and the transgenes GLUT1 and ASS1. In some embodiments, the genetically engineered immune cells comprise T or NK cells coexpressing the ACTR and the transgenes GLUT1 and PSPH. Alternatively, the genetically engineered immune cells disclosed herein may not express any chimeric receptor polypeptides.

In some embodiments, the genetically engineered immune cells, which may express or overly express at least two metabolism modulating polypeptides as disclosed herein, may be derived from tumor-infiltrating lymphocytes (TILs). Expression or overexpression of the metabolism modulating polypeptide may enhance the anti-tumor activity or the TILs in the TME.

In some embodiments, the genetically engineered immune cells, which may overly express at least two metabolism modulating polypeptides as disclosed herein, may be derived from tumor-infiltrating lymphocytes (TILs). Overexpression of the metabolism modulating polypeptides may enhance the anti-tumor activity or the TILs in tumor microenvironment. In a specific embodiment TILs are selected that are reactive/target to a specific peptide presented an MHC complex.

The TILs and/or T cells expressing genetically modified TCRs may target a peptide-MHC complex, in which the peptide may be derived from a pathogen, a tumor antigen, or an autoantigen. Some examples are provided in Table 15 below.

Any of the CAR constructs disclosed herein or an antibody to be co-used with ACTR T cells may also target any of the peptide in such peptide/MHC complex.

TABLE 15
Exemplary Peptide-MHC Targets

| Targets | Indications |
|---|---|
| NY-ESO-1 | Sarcoma, MM |
| MAGE-A10 | NSCLC, Bladder, HNSCC |
| MAGE-A4 | Sarcomas, others |
| PMEL | Melanoma |
| WT-1 | Ovarian |
| AFP | HCC |
| HPV-16 E6 | Cervical |
| HPV-16 E7 | Cervical |

In further embodiments of the invention the genetically engineered immune cell comprises a nucleic acid or nucleic acid set, which collectively comprises a first nucleotide sequence encoding one the at least two metabolism modulating polypeptides; a second nucleotide sequence encoding the other one of the at least two metabolism modulating polypeptides; and a third nucleotide sequence encoding the chimeric receptor polypeptide. In some instances, such metabolism modulating polypeptides are identical to an endogenous protein of the immune cell. Introducing additional copies of the coding sequences of such polypeptides into the immune cell would enhance the expression level of such polypeptide(s) (i.e., overly expressed) as relative to the native counterpart. In some instances, the at least two metabolism modulating polypeptides to be introduced into the immune cells are heterologous to the immune cell, i.e., do not exist or are not expressed (i.e., at a measurable level, e.g., by western blotting/immuoblotting) in the immune cell. Such a heterologous metabolism modulating polypeptides described herein may be a naturally-occurring protein not expressed in the immune cell in nature (e.g., from a different species, or from a different cell type of the same species). Alternatively, such heterologous metabolism modulating polypeptides may be a variant of a native protein, such as those described herein. In some examples, the exogenous (i.e., not native to the immune cells) copy of the coding nucleic acid may exist extra-chromosomally. In other examples, the exogenous copy of the coding sequence may be integrated into the chromosome of the immune cell, and may be located at a site that is different from the native locus of the endogenous gene.

The genetically engineered immune cells, when expressing a chimeric receptor polypeptide as disclosed herein, can recognize and inhibit target cells, either directly (e.g., by CAR-expressing immune cells) or via an Fc-containing therapeutic agents such as an antitumor antibodies (e.g., by ACTR-expressing immune cells). Given their expected high proliferation rate, bioactivity, and/or survival rate in low glucose, low amino acid, low pH, and/or hypoxic environments (e.g., in a TME), the genetically engineered immune cells such as T cell and NK cells would be expected to have higher therapeutic efficacy relative to chimeric receptor polypeptide T or NK cells that do not express or express a lower level or less active form of the metabolism modulating polypeptides.

To construct the immune cells that express at least two metabolism modulating polypeptides and optionally the chimeric receptor polypeptide described herein, expression vectors may be created via conventional methods as described in the present invention and introduced into immune cells. For example, nucleic acids encoding at least two metabolism modulating polypeptides and/or the chimeric receptor polypeptides may be cloned into one or two suitable expression vectors, such as a viral vector or a non-viral vector in operable linkage to a suitable promoter. In some instances, each of the coding sequences for the chimeric receptor polypeptide and the at least two metabolism modulating polypeptides are on two separate nucleic acid molecules and can be cloned into two separate vectors, which may be introduced into suitable immune cells simultaneously or sequentially. In other embodiments, the coding sequences for the chimeric receptor polypeptide and at least two metabolism modulating polypeptides are on one nucleic acid molecule and can be cloned into one vector. Accordingly, it is one embodiment that the immune cell comprises the nucleic acid, which comprises a first nucleotide sequence, a second nucleotide sequence and a third nucleotide sequence.

The coding sequences of the chimeric receptor polypeptide and at least two metabolism modulating polypeptides may be in operable linkage to two distinct promoters such that the expression of the two polypeptides is controlled by different promoters. In some instances, the coding sequences of the chimeric receptor polypeptide and three separate metabolism modulating polypeptides may be in operable linkage to two or three distinct promoters such that the expression of the four polypeptides is controlled by different promoters. Alternatively, the coding sequences of the chimeric receptor polypeptide and three separate metabolism modulating polypeptides may be in operably linkage to one promoter such that the expression of the two polypeptides is controlled by a single promoter. Suitable sequences may be inserted between the coding sequences of the three or more metabolism modulating polypeptides so that three or more separate polypeptides can be translated from a single mRNA molecule. Such sequences, for example, IRES or ribosomal skipping site, are well known in the art. A variety of promoters can be used for expression of the at least two metabolism modulating polypeptides and/or the chimeric receptor polypeptides described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, the human EF1-alpha promoter, or herpes simplex tk virus promoter. Additional promoters for expression of the at least two metabolism modulating polypeptides and/or the chimeric receptor polypeptides include any constitutively active promoter in an immune cell. Alternatively, any regulatable/inducible promoter may be used, such that its expression can be modulated within an immune cell. Suitable induction systems are known in the art, see, e.g., Kallunki et al. (Cells, 8(8): 796 (2019)).

Accordingly, it is one embodiment that the nucleic acid further comprises a fourth nucleotide sequence located between the first nucleotide sequence and the second nucleotide sequence, wherein the fourth nucleotide sequence encodes a ribosomal skipping site, an internal ribosome entry site (IRES), or a promoter. In other embodiments, the nucleic acid further comprises a fourth nucleotide sequence located between the second nucleotide sequence and the third nucleotide sequence, wherein the fourth nucleotide sequence encodes a ribosomal skipping site, an internal ribosome entry site (IRES), or a promoter. In some embodiments, the nucleic acid further comprises a fourth and a fifth nucleotide sequence wherein the fourth nucleotide sequence is located between the first nucleotide sequence and the second nucleotide sequence, wherein the fifth nucleotide sequence is located between the second nucleotide sequence and the third nucleotide sequence, wherein the fourth and the fifth nucleotide sequence encodes a ribosomal skipping site, an internal ribosome entry site (IRES), or a promoter. In a preferred embodiment, the fourth and fifth nucleotide sequence is a ribosomal skipping site, preferably P2A or T2A. In other embodiments, the fourth nucleotide sequence is a P2A and the fifth nucleotide sequence is a T2A.

Non-limiting examples of immune cells expressing at least two metabolism modulating polypeptides as described above and a chimeric receptor polypeptide comprises in 5′ to 3′ direction a first nucleotide sequence encoding the chimeric receptor polypeptide (ACTR or CAR); a second nucleotide sequence encoding a ribosomal skipping site P2A; a third nucleotide sequence encoding one of the metabolism modulating polypeptide; a fourth nucleotide sequence encoding a ribosomal skipping site T2A; and a fifth nucleotide sequence encoding the second of the metabolism modulating polypeptide In a preferred embodiment, the immune cell comprising two metabolism modulating polypeptides are selected from the group consisting of: (a) GOT2 and TIGAR; (b) GOT2 and GLUT1; (c) GOT2 and PDK1; (d) TIGAR and GLUT1; (e) PDK1 and CTH; (f) CTH and PSPH; (g) GLUT1 and ASS1; and (h) GLUT1 and PSPH.

The immune cells described herein may further comprise three metabolism modulating polypeptides selected from GOT2, GLUT1, LDHA, PDK1, TIGAR, CTH, ASS1 and PSPH. In further embodiments of the invention the genetically engineered immune cell comprises a nucleic acid or nucleic acid set, which collectively comprises a first nucleotide sequence encoding the first metabolism modulating polypeptide; a second nucleotide sequence encoding the second metabolism modulating polypeptide; a third nucleotide sequence encoding the third metabolism modulating polypeptide; and a fourth nucleotide sequence encoding the chimeric receptor polypeptide. In some instances, each of the coding sequences for the chimeric receptor polypeptide and at least two metabolism modulating polypeptides are on separate nucleic acid molecules and can be cloned into separate vectors, which may be introduced into suitable immune cells simultaneously or sequentially. In some instances, each of the coding sequences for the chimeric receptor polypeptide and at least two metabolism modulating polypeptides are on separate nucleic acid molecules and can be cloned into one vector which may be introduced into suitable immune cells. In some embodiments the immune cell comprises a nucleic acid, which comprises a first nucleotide sequence encoding the chimeric receptor polypeptide, a second nucleotide sequence, a third nucleotide sequence and a fourth nucleotide sequence each encoding one of the metabolism modulating (e.g., CAR-polypeptide 1-polypeptide 2-polypeptide 3). In some embodiments, the nucleic acid further comprises a fifth nucleotide sequence wherein the fifth nucleotide sequence is located between the first nucleotide sequence and the second nucleotide sequence, wherein the fifth nucleotide sequence is a ribosomal skipping site, an internal ribosome entry site (IRES), or a promoter (e.g., CAR-P2A-polypeptide 1-polypeptide 2-polypeptide 3). In some embodiments, the nucleic acid further comprises a sixth nucleotide sequence, wherein the sixth nucleotide sequence is located between the second nucleotide sequence and the third nucleotide sequence, wherein the sixth nucleotide sequence is a ribosomal skipping site, an internal ribosome entry site (IRES), or a promoter (e.g., CARP2A-polypeptide 1-P2A-polypeptide 2-polypeptide 3). In some embodiments, the nucleic acid further comprises and a seventh nucleotide sequence, wherein the seventh nucleotide sequence is located between the third nucleotide sequence and the fourth nucleotide sequence, wherein the seventh nucleotide sequence/is a ribosomal skipping site, an internal ribosome entry site (IRES), or a promoter (CAR-P2A-polypeptide 1-polypeptide 2-P2A-polypeptide 3). Additional descriptions are provided below.

In further embodiments the nucleic acid or nucleic acid set is comprised within one or more viral vectors. The nucleic acids and the vector(s) may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding at least two metabolism modulating polypeptides and/or the chimeric receptor polypeptides. The synthetic linkers may contain nucleic acid sequences that correspond to a particular restriction site in the vector. The selection of expression vectors/plasmids/viral vectors would depend on the type of immune cells for expression of the at least two metabolism modulating polypeptides described herein and/or the chimeric receptor polypeptides, but should be suitable for integration and replication in eukaryotic cells. An exemplary embodiment is a method of modifying the metabolism of immune cells, comprising transfecting immune cells transiently or stably with the vector or vector set and collecting immune cells transfected with the vector or vector set.

Additionally, the vector may contain, for example, some or all of the following: a selectable marker gene, such as the neomycin gene or the kanamycin gene for selection of stable or transient transfectants in immune cells; enhancer/promoter sequences from the immediate early gene of human CMV for high levels of transcription; intron sequences from the human EF1-alpha gene, transcription termination and RNA processing signals from SV40 for mRNA stability; SV40 or polyomavirus origins of replication and ColE1 for proper episomal replication; internal ribosome binding sites (IRESs), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase or an inducible caspase such as iCasp9), and reporter gene(s) for assessing expression of the metabolism modulating polypeptide and/or the chimeric receptor polypeptide.

In one specific embodiment, such vectors also include a suicide gene. As used herein, the term “suicide gene” refers to a gene that causes the cell expressing the suicide gene to die. The suicide gene can be a gene (e.g., HSV thymidine kinase) that confers sensitivity to an agent, e.g., a drug (e.g., ganciclovir for HSV thymidine kinase), upon the cell in which the gene is expressed, and causes the cell to die when the cell is contacted with or exposed to the agent. Suicide genes are known in the art (see, for example, Springer, C. J. (Suicide Gene Therapy: Methods and Reviews, Humana Press (2004)) and include, for example, the Herpes Simplex Virus (HSV) thymidine kinase (TK) gene, cytosine deaminase, purine nucleoside phosphorylase, nitroreductase, and caspases such as caspase 8 or caspase-9 (iCasp9).

Suitable vectors and methods for producing vectors containing transgenes are well known and available in the art. Examples of the preparation of vectors for expression of metabolism modulating polypeptides and/or chimeric receptor polypeptides can be found, for example, in US 2014/0106449, herein incorporated in its entirety by reference.

Any of the vectors comprising a nucleic acid sequence that encodes the metabolism modulating polypeptides and/or a chimeric receptor polypeptide described herein is also within the scope of the present invention. Such a vector, or the sequence encoding such metabolism modulating polypeptides and/or a chimeric receptor polypeptide contained therein, may be delivered into immune cells such as immune cells by any suitable method. Methods of delivering vectors to immune cells are well known in the art and may include DNA electroporation, RNA electroporation, transfection using reagents such as liposomes, or viral transduction (e.g., retroviral transduction such as lentiviral or gamma-retroviral transduction).

In some embodiments, the vectors for expression of the at least two metabolism modulating polypeptides and/or the chimeric receptor polypeptides are delivered to immune cells by viral transduction (e.g., retroviral transduction such as lentiviral or gamma-retroviral transduction). Exemplary viral methods for delivery include, but are not limited to, recombinant retroviruses (see, e.g., WO 90/07936; WO 94/03622; WO 93/25698; WO 93/25234; WO 93/11230; WO 93/10218; and WO 91/02805; U.S. Pat. Nos. 5,219,740 and 4,777,127; GB 2,200,651; and EP 0345242), alphavirus-based vectors, and adeno-associated virus (AAV) vectors (see, e.g., WO 94/12649, WO 93/03769; WO 93/19191; WO 94/28938; WO 95/11984; and WO 95/00655). In some embodiments, the vectors for expression of the at least two metabolism modulating polypeptides and/or the chimeric receptor polypeptides are retroviruses. In a preferred embodiment, the vectors are lentiviruses.

Examples of references describing retroviral transduction include U.S. Pat. No. 5,399,346; Mann et al. (Cell, 33(1): 153-159 (1983)); U.S. Pat. Nos. 4,650,764; 4,980,289; Markowitz et al. (J Virol, 62(4): 1120-1124 (1988)); U.S. Pat. No. 5,124,263; WO 95/07358 and Kuo et al. (Blood, 82(3): 845-852 (1993)). WO 95/07358 describes high efficiency transduction of primary B lymphocytes. See also WO 2016/040441A1, all incorporated by reference herein for the purpose and subject matter referenced herein.

In examples in which the vectors encoding at least two metabolism modulating polypeptides and/or, the chimeric receptor polypeptides are introduced to the immune cells using a viral vector, viral particles that are capable of infecting the immune cells and carry the vector may be produced by any method known in the art and can be found, for example in WO 91/02805A2, WO 98/09271A1, and U.S. Pat. No. 6,194,191. The viral particles are harvested from the cell culture supernatant and may be isolated and/or purified prior to contacting the viral particles with the immune cells.

In some embodiments, RNA molecules encoding the at least two metabolism modulating polypeptides and/or the chimeric receptor polypeptides as described herein may be prepared by a conventional method (e.g., in vitro transcription) and then introduced into suitable immune cells, e.g., those described herein, via known methods, e.g., Rabinovich et al. (Human Gene Therapy, 17(10): 1027-1035 (2006)).

The disclosure also relates to a nucleic acid of the present invention. In some instances, the nucleic acid encoding at least two metabolism modulating polypeptides and the nucleic acid encoding a suitable chimeric receptor polypeptide may be cloned into separate expression vectors, which may be introduced into suitable immune cells concurrently or sequentially. For example, an expression vector (or an RNA molecule) for expressing at least two metabolism modulating polypeptides may be introduced into immune cells first and the transfected immune cells expressing at least two metabolism modulating polypeptides may be isolated and cultured in vitro. In another example, an expression vector (or an RNA molecule) expressing a suitable chimeric receptor polypeptide can then introduced into the immune cells expressing at least two metabolism modulating polypeptides where all three polypeptides can be isolated. In another example, expression vectors (or RNA molecules) each for expressing at least two metabolism modulating polypeptides and the chimeric receptor polypeptide can be introduced into immune cells simultaneously and transfected immune cells expressing all three polypeptides can be isolated via routine methodology. In some instances, the nucleic acid(s) encoding at least two metabolism modulating polypeptides and/or the chimeric receptor polypeptide may be delivered into immune cells via transposons (e.g., piggybac). In some instances, the encoding nucleic acid(s) may be delivered into immune cells via gene editing, for example, by CRISPR, TALEN, zinc-finger nuclease (ZFN), or meganucleases.

In other instances, the nucleic acid encoding at least two metabolism modulating polypeptides and the nucleic acid encoding the chimeric receptor polypeptide may be cloned into the same expression vector. Polynucleotides (including vectors in which such polynucleotides are operably linked to at least one regulatory element) for expression of the chimeric receptor polypeptide and at least two metabolism modulating polypeptides are also within the scope of the present disclosure. Non-limiting examples of useful vectors of the disclosure include viral vectors such as, e.g., retroviral vectors including gamma retroviral vectors and lentiviral vectors, and adeno-associated virus vectors (AAV vectors).

In some instances, the nucleic acid described herein may comprise three coding sequences, one encoding a chimeric receptor polypeptide as described herein, and the other at least two encoding metabolism modulating polypeptides. The nucleic acid comprising the coding sequences described herein may be configured such that the coding sequences encoding the metabolism modulating polypeptides can be expressed as independent (and physically separate) polypeptides. To achieve this goal, the nucleic acid described herein may contain a fourth, optionally fifth or sixth, nucleotide sequence located between the coding sequences for the metabolism modulating polypeptides. This fourth, fifth and/or sixth nucleotide sequence may, for example, encode a ribosomal skipping site. A ribosomal skipping site is a sequence that impairs normal peptide bond formation. This mechanism results in the translation of additional open reading frames from one messenger RNA. This fourth, fifth and/or sixth nucleotide sequence may, for example, encode a P2A, T2A, or F2A peptide (see, for example, Kim, Lee et al. (PLOS One, 6(4): e18556 (2011)). See Table 16 below.

TABLE 16
Exemplary Ribosomal Skipping Peptides

| Ribosomal | | SEQ |
|---|---|---|
| Skipping Site | Sequence | ID NO |
| P2A | ATNFSLLKQAGDVEENPGP | 74 |
| T2A | EGRGSLLTCGDVEENPGP | 88 |
| E2A | QCTNYALLKLAGDVESNPGP | 89 |
| F2A | AVKQTLNFDLLKLAGDVESNPGP | 90 |

The ribosomal skipping peptides include derivatives of P2A, T2A, E2A and F2A that contain one or more conservative substitutions and/or N- or C-terminal deletions of one or two amino acids.

In another embodiment, the fourth nucleotide sequence may encode an internal ribosome entry site (IRES). An IRES is an RNA element that allows translation initiation in an end-independent manner, also permitting the translation of additional open reading frames from one messenger RNA. Alternatively, the fourth nucleotide sequence may encode a promoter controlling the expression of the second polypeptide and/or the third polypeptide. The fourth nucleotide sequence may also encode more than one ribosomal skipping sequence, IRES sequence, additional promoter sequence, or a combination thereof. An exemplar IRES sequence is provided below as SEQ ID NO: 91.

GAGGGCCCGGAAACCTGGCCCTGTCTTCTTGACGAGCATTCCTAGGGGTC
TTTCCCCTCTCGCCAAAGGAATGCAAGGTCTGTTGAATGTCGTGAAGGAA
GCAGTTCCTCTGGAAGCTTCTTGAAGACAAACAACGTCTGTAGCGACCCT
TTGCAGGCAGCGGAACCCCCCACCTGGCGACAGGTGCCTCTGCGGCCAAA
AGCCACGTGTATAAGATACACCTGCAAAGGCGGCACAACCCCAGTGCCAC
GTTGTGAGTTGGATAGTTGTGGAAAGAGTCAAATGGCTCTCCTCAAGCGT
ATTCAACAAGGGGCTGAAGGATGCCCAGAAGGTACCCCATTGTATGGGAT
CTGATCTGGGGCCTCGGTGCACATGCTTTACATGTGTTTAGTCGAGGTTA
AAAAAACGTCTAGGCCCCCCGAACCACGGGGACGTGGTTTTCCTTTGAAA
AACACGATGATAA

The nucleic acid may also include additional coding sequences (including, but not limited to, fifth and sixth coding sequences) encoding further metabolism modulating polypeptides and may be configured such that the polypeptides encoded by the additional coding sequences are expressed as further independent and physically separate polypeptides. To this end, the additional coding sequences may be separated from other coding sequences for a polypeptide by one or more nucleotide sequences encoding one or more ribosomal skipping sequences, IRES sequences, or additional promoter sequences.

In some examples, the nucleic acid (e.g., an expression vector or an RNA molecule as described herein) may comprise coding sequences for at least two metabolism modulating polypeptides and a suitable chimeric receptor polypeptide, the coding sequences (for example, three), in any order, being separated by a fourth nucleotide sequence coding for a P2A peptide (e.g., SEQ ID NO: 74). As a result, three separate polypeptides, at least two metabolism modulating polypeptides and the chimeric receptor, can be produced from such a nucleic acid, wherein at least one P2A portion (e.g., SEQ ID NO: 74) is linked to the upstream polypeptide (encoded by the upstream coding sequence) and residue P from the P2A peptide is linked to the downstream polypeptide (encoded by the downstream coding sequence). In some examples, the chimeric receptor polypeptide is the upstream one and at least two metabolism modulating polypeptides are the downstream one. In other examples, the at least two metabolism modulating polypeptides are the upstream one and the chimeric receptor polypeptide is the downstream one.

In some embodiments, the nucleic acid (e.g., an expression vector or an RNA molecule as described herein) may comprise coding sequences of at least two metabolism modulating polypeptides (e.g., those described herein) and a suitable ACTR or CAR polypeptide, the coding sequences, in any order, being separated by a fourth nucleotide sequence coding for a P2A peptide (e.g., SEQ ID NO: 74). As a result, at least three separate polypeptides, the metabolism modulating polypeptides and the ACTR or CAR) can be produced from such a nucleic acid, wherein the P2A portion (e.g., SEQ ID NO: 74) is linked to the upstream polypeptide (encoded by the upstream coding sequence) and residue P from the P2A peptide is linked to the downstream polypeptide (encoded by the downstream coding sequence). In some embodiments, the ACTR or CAR polypeptide is the upstream one and the at least two metabolism modulating polypeptides are the downstream one. In other embodiments, the at least two metabolism modulating polypeptides are the upstream one and the ACTR or CAR polypeptide is the downstream one.

In some examples, the nucleic acid described above may further encode a linker (e.g., a GSG linker) between two segments of the encoded sequences, for example, between the upstream polypeptide and the P2A peptide.

In specific examples, the nucleic acid described herein is configured such that it expresses at least three separate metabolism modulating polypeptides in the immune cell to which the nucleic acid is transfected: (i) the first polypeptide that contains, from the N-terminus to the C-terminus, a suitable CAR (e.g., enlisted in Table 11-Table 13 or SEQ ID NO: 86-SEQ ID NO: 87 or SEQ ID NO: 94), a peptide linker (e.g., the GSG linker), and the P2A ribosomal skipping peptide (SEQ ID NO: 74) segment derived from the P2A peptide; (ii) a second polypeptide that contains, from the N-terminus to the C-terminus, the P residue derived from the P2A peptide and the metabolism modulating polypeptide (e.g., any of SEQ ID NOs: SEQ ID NO: 75-SEQ ID NO: 82); and (iii) a third a second polypeptide that optionally, contains, from the N-terminus to the C-terminus, the P residue derived from the P2A peptide and the second metabolism modulating polypeptide (e.g., any of SEQ ID NOs: SEQ ID NO: 75-SEQ ID NO: 82).

In specific examples, the nucleic acid described herein is configured such that it expresses at least three separate metabolism modulating polypeptides in the immune cell to which the nucleic acid is transfected: (i) the first polypeptide that contains, from the N-terminus to the C-terminus, a suitable ACTR (see Table 10, a peptide linker (e.g., the GSG linker), and the P2A ribosomal skipping peptide (SEQ ID NO: 74) segment derived from the P2A peptide; (ii) a second polypeptide that contains, from the N-terminus to the C-terminus, the P residue derived from the P2A peptide and the metabolism modulating polypeptide (e.g., any of SEQ ID NO: 75 to SEQ ID NO: 82); and (iii) a third polypeptide that optionally, contains, from the N-terminus to the C-terminus, the P residue derived from the P2A peptide and the second metabolism modulating polypeptide (e.g., any of SEQ ID NO: 75 to SEQ ID NO: 82). In some instances, additional polypeptides of interest may also be introduced into the—immune cells. Specific embodiments relate to a vector or vector set comprising the nucleic acid or nucleic acid set. In some embodiments, the vector or vector set is comprised within a viral vector wherein the viral vector is lentiviral or retroviral. The method of producing viral particles using the viral vector comprising the nucleic acid or nucleic acid are well known in the art and have been described below. Specific embodiments relate to a method of producing viral particles, wherein (a) providing host cells stably transfected with the nucleic acid or nucleic acid set of or the vector or vector set; (b) growing the stably transfected host cells in a cell culture medium under conditions allowing for producing viral particles by the host cells; and (c) harvesting the viral particles from the cell culture medium. Some embodiments relate to the viral particle produced. Exemplary embodiments relate to a method of producing an immune cell that expresses the metabolism modulating polypeptides and the chimeric receptor polypeptide, comprising incubating immune cells with the viral particle under conditions allowing for infection of immune cells by the viral particle. In preferred embodiment, an immune cell is produced by the method described herein.

In a preferred embodiment, the immune cell co-expresses at least two metabolism modulating polypeptides (e.g., any of SEQ ID NO: 75 to SEQ ID NO: 82), and/or the chimeric receptor polypeptide.

Following introduction into the immune cells a vector encoding at least two metabolism modulating polypeptides and/or the chimeric receptor polypeptides provided herein, or the nucleic acid encoding the chimeric receptor polypeptide and/or metabolism modulating polypeptides, the cells may be cultured under conditions that allow for expression of the polypeptides and/or the chimeric receptor polypeptide (e.g., under a regulatable promoter, the immune cells may be cultured in conditions wherein the regulatable promoter is activated). In some embodiments, the promoter is an inducible promoter and the immune cells are cultured in the presence of the inducing molecule or in conditions in which the inducing molecule is produced. Determining whether the at least two metabolism modulating polypeptides and/or the chimeric receptor polypeptide is expressed will be evident to one of skill in the art and may be assessed by any known method, for example, mRNA by quantitative reverse transcriptase PCR (qRT-PCR) or protein by methods including Western/immuno blotting, fluorescence microscopy, and flow cytometry.

Alternatively, expression of the chimeric receptor polypeptide may take place in vivo after the immune cells are administered to a subject. As used herein, the term “subject” refers to any mammal such as a human, monkey, mouse, rabbit, or domestic mammal. For example, the subject may be a primate. In a preferred embodiment, the subject is human.

Alternatively, expression of at least two metabolism modulating polypeptides, and/or a chimeric receptor polypeptide in any of the immune cells disclosed herein can be achieved by introducing RNA molecules. Such RNA molecules can be prepared by in vitro transcription or by chemical synthesis. The RNA molecules can then be introduced into suitable immune cells (e.g., αβ or γδ T or NK cells) by, e.g., electroporation. For example, RNA molecules can be synthesized and introduced into immune cells following the methods described in Rabinovich, Komarovskaya et al. (Human Gene Therapy, 17(10): 1027-1035 (2006)) and WO 2013/040557.

In certain embodiments, a vector(s) or RNA molecule(s) comprising at least two metabolism modulating polypeptides and/or the chimeric receptor polypeptide may be introduced to the immune cells in vivo. As a non-limiting example, this may be accomplished by administering a vector or RNA molecule described herein directly to the subject (e.g., through intravenous administration). An exemplary embodiment is a method of modifying the metabolism of immune cells, comprising transfecting immune cells transiently or stably with the vector or vector set and collecting immune cells transfected with the vector or vector set. In some other embodiments, the method for generating modified immune cells in vivo comprises administering to a subject in need thereof the nucleic acid or nucleic acid set, the vector or vector set, or the viral particles described herein. A preferred embodiment is a population of genetically engineered immune cells for use in inhibiting cells expressing a target antigen in a subject. In other embodiments, the population of genetically engineered immune cells for use, wherein at least some of the cells expressing the target antigen are located in a low-glucose environment.

Methods for preparing immune cells expressing at least two metabolism modulating polypeptides and/or the chimeric receptor polypeptides described herein may also comprise activating the immune cells ex vivo. Activating an immune cell means stimulating an immune cell into an activated state in which the cell may be able to perform effector functions. Methods of activating an immune cell will depend on the type of immune cell used for expression of the at least two metabolism modulating polypeptides and/or chimeric receptor polypeptides. For example, T cells may be activated ex vivo in the presence of one or more molecules including, but not limited to: an anti-CD3 antibody, an anti-CD28 antibody, IL-2, phytohemoagglutinin, engineered artificial stimulatory cells or particles, or a combination thereof. The engineered artificial stimulatory cells may be artificial antigen-presenting cells as known in the art. See, e.g., Neal et al. (J Immunol Res Ther, 2(1): 68-79 (2017)) and Turtle and Riddell (Cancer journal (Sudbury, Mass.), 16(4): 374-381 (2010)), the relevant disclosures of each of which are hereby incorporated by reference for the purpose and subject matter referenced herein.

In other examples, NK cells may be activated ex vivo in the presence of one or more molecules such as a 4-1BB ligand, an anti-4-1BB antibody, IL-2, IL-15, an anti-IL-15 receptor antibody, IL-12, IL-21, K562 cells, and/or engineered artificial stimulatory cells or particles. In some embodiments, the immune cells of the present invention and described herein are activated ex vivo prior to administration to a subject. Determining whether an immune cell is activated will be evident to one of skill in the art and may include assessing expression of one or more cell surface markers associated with cell activation, expression or secretion of cytokines, and cell morphology. Further, the methods for preparing immune cells described herein may comprise expanding the immune cells ex vivo. Expanding immune cells may involve any method that results in an increase in the number of cells expressing metabolism modulating polypeptides and/or chimeric receptor polypeptides, for example, allowing the immune cells to proliferate or stimulating the immune cells to proliferate. Methods for stimulating expansion of immune cells will depend on the type of immune cell used and will be evident to one of skill in the art.

In some embodiments, the immune cells expressing at least two metabolism modulating polypeptides and/or the chimeric receptor polypeptides are expanded and activated ex vivo prior to administration of the cells to the subject. Immune cell activation and expansion may be used to allow integration of a viral vector into the genome and expression of the gene encoding the at least two metabolism modulating polypeptides and/or the chimeric receptor polypeptide as described herein. If mRNA electroporation is used, no activation and/or expansion may be required, although electroporation may be more effective when performed on activated cells. In some instances, the at least two metabolism modulating polypeptides and/or a chimeric receptor polypeptide is transiently expressed in a suitable immune cell (e.g., for 3-5 days). Transient expression may be advantageous, if there is a potential toxicity and should be helpful in initial phases of clinical testing for possible side effects of immunotherapy using the genetically engineered immune/hematopoietic cells described herein.

The genetically engineered immune cells (e.g., NK cells or αβ or γδ T cells) of the present invention disclosed herein may be used in immunotherapy against various disorders, for example, cancer, infectious diseases, and autoimmune diseases. Accordingly, another embodiment of the present invention is a method for inhibiting cells expressing a target antigen in a subject, the method comprising administering to a subject in need thereof a population of the genetically engineered immune cells set forth herein or a pharmaceutical composition comprising a population of the genetically engineered immune cells set forth herein.

Any of the immune cells of the present invention may be mixed preferably with a pharmaceutically acceptable carrier to form a pharmaceutical composition, which is also within the scope of the present disclosure. Therefore, a pharmaceutical composition, comprising a genetically engineered immune cell of the invention is another embodiment.

The phrase “pharmaceutically acceptable”, as used in connection with compositions of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the U.S. federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The pharmaceutical compositions of the disclosure may also contain one or more additional active compounds as necessary for the particular indication being treated, preferably those with complementary activities that do not adversely affect each other. Nonlimiting examples of possible additional active compounds include, e.g., IL-2 as well as various agents known in the field and listed in the discussion of combination treatments, below.

### IV. Immunotherapy Using the Genetically Engineered Hematopoietic Cells Described Herein

The genetically engineered immune/hematopoietic cells (e.g., hematopoietic stem cells, immune cells, such as NK cells or T cells) disclosed herein may be used in immunotherapy against various disorders, for example, cancer, infectious diseases, and autoimmune diseases. Accordingly, another embodiment of the present invention is a method for inhibiting cells expressing a target antigen in a subject, the method comprising administering to a subject in need thereof a population of the genetically engineered immune cells set forth herein or a pharmaceutical composition comprising a population of the genetically engineered immune cells set forth herein.

### A. Combined Immunotherapy of Genetically Engineered Immune Cells Expressing ACTR Polypeptides and Fc-Containing Therapeutic Agents

The exemplary ACTR polypeptides of the present disclosure confer antibody-dependent cell cytotoxicity (ADCC) capacity to T lymphocytes and enhance ADCC in NK cells. When the receptor is engaged by an antibody bound to cells, it triggers T-cell activation, sustained proliferation and specific cytotoxicity against the bound cells.

The degree of affinity of CD16 for the Fc portion of Ig is a critical determinant of ADCC and thus to clinical responses to antibody immunotherapy. The CD16 with the V158 polymorphism which has a higher binding affinity for Ig and mediates superior ADCC relative to CD16 with the F158 polymorphism was selected as an example. Although the F158 receptor has lower potency than the V158 receptor in induction of T cell proliferation and ADCC, the F158 receptor may have lower in vivo toxicity than the V158 receptor making it useful in some clinical contexts.

At least two metabolism modulating polypeptides co-expressed with an ACTR polypeptides in immune cells would facilitate cell-based immune therapy such as T-cell therapy or NK-cell therapy by allowing the cells to grow and/or function effectively in a low glucose, low amino acid, low pH, and/or hypoxic environment. Antibody-directed cytotoxicity could be stopped whenever required by simple withdrawal of antibody administration. Clinical safety can be further enhanced by using mRNA electroporation to express at least two metabolism modulating polypeptides and/or the ACTR polypeptides transiently, to limit any potential autoimmune reactivity.

Thus, in one embodiment, the disclosure provides a method for enhancing efficacy of an antibody-based immunotherapy of a cancer in a subject in need thereof, which subject is being treated with an Fc-containing therapeutic agent such as a therapeutic antibody, which can bind to antigen-expressing cells. The Fc-containing therapeutic agent contains an Fc portion, for example, a human or humanized Fc portion, which can be recognized and bound by the Fc-binding portion (e.g., the extracellular domain of human CD16A) of the ACTR expressed on the engineered immune cells. Exemplary ACTR constructs are provided in Table 10 above.

The methods described herein may comprise introducing into the subject a therapeutically effective amount an antibody and a therapeutically effective amount of the genetically engineered immune cells (e.g., T or NK cells), of the present invention. The subject (e.g., a human patient such as a human cancer patient) has been treated or is being treating with an Fc-containing therapeutic agent specific to a target antigen. A target antigen may be any molecule that is associated with a disease or condition, including, but are not limited to, tumor antigens, pathogenic antigens (e.g., bacterial, fungal or viral), or antigens present on diseased cells, such as those described herein.

In the context of the present disclosure insofar as it relates to any of the disease conditions recited herein, the terms “treat”, “treatment”, and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

As used herein the term “therapeutically effective” applied to dose or amount refers to that quantity of a compound or pharmaceutical composition that is sufficient to result in a desired activity upon administration to a subject in need thereof. Note that when a combination of active ingredients is administered (e.g., a first pharmaceutical composition comprising an antibody, and a second pharmaceutical composition comprising a population of the genetically modified immune cells (e.g., T or NK cells) that express at least two metabolism modulating polypeptide and/or an antibody-coupled T-cell receptor (ACTR) construct, the effective amount of the combination may or may not include amounts of each ingredient that would have been effective if administered individually. Within the context of the present disclosure, the term “therapeutically effective” refers to that quantity of a compound or pharmaceutical composition that is sufficient to delay the manifestation, arrest the progression, relieve or alleviate at least one symptom of a disorder treated by the methods of the present disclosure.

Immune cells (e.g., T and NK cells) expressing at least two metabolism modulating polypeptides and an ACTR polypeptides described herein are useful for enhancing ADCC in a subject, for enhancing the efficacy of an antibody-based immunotherapy and/or for enhancing growth and/or proliferation of the genetically engineered immune cells in a low-glucose environment. In some embodiments, the subject is a mammal, such as a human, monkey, mouse, rabbit, or domestic mammal. In some embodiments, the subject is a human. In some embodiments, the subject is a human cancer patient. In some embodiments, the subject has been treated or is being treated with any of the therapeutic antibodies described herein.

To practice the method described herein, an effective amount of the immune cells, described herein and an effective amount of an antibody, or compositions thereof may be administered to a subject in need of the treatment via a suitable route, such as intravenous administration. As used herein, an effective amount refers to the amount of the respective agent (e.g., the genetically engineered immune cells of the present disclosure and the ACTR polypeptide, antibodies, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender, sex, and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject in need of treatment is a human. In some embodiments, the subject in need of treatment is a human cancer patient. In some embodiments, the subject in need of treatment suffers from one or more pathogenic infections (e.g., viral, bacterial, and/or fungal infections).

In some embodiments, the genetically engineered immune cells described herein are administered to a subject in an amount effective in enhancing ADCC activity by least 20% and/or by at least 2-fold, e.g., enhancing ADCC by 50%, 80%, 100%, 2-fold, 5-fold, 10-fold, 20-fold, 50-fold, 100-fold, or more. The immune cells are co-administered with an Fc-containing therapeutic agent such as a therapeutic antibody in order to target cells expressing the antigen to which the Fc-containing therapeutic agent binds. In some embodiments, more than one Fc-containing therapeutic agents, such as more than one antibody can be co-used with the immune cells.

Antibody-based treatment may therefore, improve the overall health status of the patient in need receiving the treatment. An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof which comprise an Fc region, mutants thereof, fusion proteins comprising an antibody portion, humanized antibodies, chimeric antibodies, diabodies, single domain antibodies (e.g., nanobodies), linear antibodies, multispecific antibodies (e.g., bispecific antibodies) and any other modified configuration of the immunoglobulin molecule that comprises an antigen recognition site of the required specificity and an Fc region, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD. IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. The antibody for use in the present disclosure contains an Fc region recognizable by the co-used ACTR-expressing immune cells. The Fc region may be a human or humanized Fc region.

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the antibodies described herein specifically bind to the corresponding target antigen or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen.

In some embodiments, an antibody as described herein has a suitable binding affinity for the target antigen (e.g., any one of the targets described herein) or antigenic epitopes thereof. The antibodies for use in the immune therapy methods described herein may bind to (e.g., specifically bind to) a target antigen of interest, or a specific region or an antigenic epitope therein. Table 4 above lists exemplary target antigens of interest and exemplary antibodies specific to such. The methods of the disclosure may be used for treatment of any cancer have been described herein. The methods of this disclosure may also be used for treating infectious diseases, which may be caused by bacterial infection, viral infection, or fungus infection. In such instances, the genetically engineered immune cells can be co-used with an Fc-containing therapeutic agent (e.g., an antibody) that targets a pathogenic antigen (e.g., an antigen associated with the bacterium, virus, or fungus that causes the infection).

### B. Immunotherapy of Genetically Engineered Immune Cells Expressing CAR Polypeptides

The genetically engineered immune cells (e.g., αβ or γδ T or NK cells) described herein, co-expressing at least two metabolism modulating polypeptides and a CAR polypeptide can be used in immune therapy such as T-cell therapy (both αβ and γδ T cells) or NK-cell therapy for inhibiting and/or killing diseased cells expressing an antigen to which the CAR polypeptide targets, directly or indirectly (e.g., via a therapeutic agent conjugated to a tag to which the CAR polypeptide binds). The at least two metabolism modulating polypeptides are preferably selected from the group consisting of: (a) GOT2 and TIGAR; (b) GOT2 and GLUT1; (c) GOT2 and PDK1; (d) TIGAR and GLUT1; (e) PDK1 and CTH; (f) CTH and PSPH; (g) GLUT1 and ASS1; and (h) GLUT1 and PSPH. Their co-expression with a CAR polypeptide in immune cells would facilitate the cell-based immune therapy by allowing the cells to grow and/or function effectively in a low glucose, low amino acid, low pH, and/or a hypoxic environment, for example, in a tumor microenvironment. Clinical safety may be further enhanced by using mRNA electroporation to express the at least two metabolism modulating polypeptides from above and/or the CAR polypeptides transiently, to limit any potential non-tumor specific reactivity.

The methods described herein may comprise introducing into the subject a therapeutically effective amount of genetically engineered immune cells s (e.g., αβ T, γδ T or NK cells), which co-express the at least two metabolism modulating polypeptides and a CAR polypeptide of the disclosure (see exemplary examples in Table 11 to Table 13 for the respective immune cells). The subject (e.g., a human patient) may additionally have been treated or is being treated with an anti-cancer or anti-infection therapy including, but not limited to, an anti-cancer therapeutic agent or anti-infection agent.

Immune cells (e.g., T and NK cells) expressing at least two metabolism modulating polypeptides and a CAR polypeptide described herein are useful for inhibiting cells expressing a target antigen and/or for enhancing growth and/or proliferation of immune cells in a low-glucose environment, a low amino acid environment, a low pH environment, and/or a hypoxic environment, for example, in a tumor microenvironment. In some embodiments, the subject is a mammal, such as a human, monkey, mouse, rabbit, or domestic mammal. In some embodiments, the subject is a human. In some embodiments, the subject is a human cancer patient. In some embodiments, the subject is a human patient suffering from an infectious disease.

To practice the method described herein, an effective amount of the genetically engineered immune cells described herein, or compositions thereof may be administered to a subject in need of the treatment via a suitable route, such as intravenous, subcutaneous and intradermal administration, preferably intravenous. As used herein, an effective amount refers to the amount of the respective agent (e.g., the NK and/or T cells expressing metabolism modulating polypeptides CAR polypeptides, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art and have been described herein.

The methods of the disclosure may be used for treatment of any cancer or any pathogen. Specific non-limiting examples of cancers which can be treated by the methods of the disclosure include, for example, lymphoma, breast cancer, gastric cancer, neuroblastoma, osteosarcoma, lung cancer, skin cancer, prostate cancer, colorectal cancer, renal cell carcinoma, ovarian cancer, rhabdomyosarcoma, leukemia, mesothelioma, pancreatic cancer, head and neck cancer, retinoblastoma, glioma, glioblastoma, thyroid cancer, hepatocellular cancer, esophageal cancer, and cervical cancer. In certain embodiments, the cancer may be a solid tumor. In certain embodiments, the cancer may be a liquid tumor. Accordingly, a preferred embodiment is a method for inhibiting cells expressing a target antigen in a subject, wherein the subject is a human patient suffering from a cancer and the target antigen is a tumor antigen; wherein the cancer is selected from the group consisting of carcinoma, lymphoma, sarcoma, blastoma, and leukemia, preferably wherein the cancer is selected from the group consisting of a cancer of B-cell origin, breast cancer, gastric cancer, neuroblastoma, osteosarcoma, lung cancer, skin cancer, prostate cancer, colon cancer, renal cell carcinoma, ovarian cancer, rhabdomyosarcoma, leukemia, mesothelioma, pancreatic cancer, head and neck cancer, retinoblastoma, glioma, glioblastoma, liver cancer, and thyroid cancer; or the cancer of B-cell origin is selected from the group consisting of B-lineage acute lymphoblastic leukemia, B-cell chronic lymphocytic leukemia, and B-cell non-Hodgkin's lymphoma.

The methods of this disclosure may also be used for treating infectious diseases, which may be caused by bacterial infection, viral infection, or fungal infection. In such instances, genetically engineered immune cells expressing a CAR polypeptide specific to a pathogenic antigen, (e.g., an antigen associated with the bacterium, virus, or fungus that causes the infection) can be used to eliminate infected cells. Specific non-limiting examples of pathogenic antigens include, but are not limited to, bacterial, viral, and/or fungal antigens.

In some embodiments, the immune cells are administered to a subject in an amount effective in inhibiting cells expressing the target antigen by least 20% and/or by at least 2-fold, e.g., inhibiting cells expressing the target antigen by 50%, 80%, 100%, 2-fold, 5-fold, 10-fold, 20-fold, 50-fold, 100-fold, or more.

Additional therapeutic agents (e.g., antibody-based immunotherapeutic agents) may be used to treat, alleviate, or reduce the symptoms of any disease or disorder for which the therapeutic agent is considered useful in a subject. The efficacy of the cell-based immunotherapy as described herein may be assessed by any method known in the art and would be evident to a skilled medical professional. For example, the efficacy of cell-based immunotherapy may be assessed by survival of the subject or tumor or cancer burden in the subject or tissue or sample thereof. In some embodiments, the immune cells are administered to a subject in need of the treatment in an amount effective in enhancing the efficacy of an cell-based immunotherapy by at least 20% and/or by at least 2-fold, e.g., enhancing the efficacy of an antibody-based immunotherapy by 50%, 80%, 100%, 2-fold, 5-fold, 10-fold, 20-fold, 50-fold, 100-fold or more, as compared to the efficacy in the absence of the immune cells expressing at least two metabolism modulating polypeptides and/or the CAR polypeptide.

In any of the compositions or methods described herein, the immune cells (e.g., NK and/or T cells) may be autologous to the subject, i.e., the immune cells may be obtained from the subject in need of the treatment, genetically engineered for expression of at least two metabolism modulating polypeptides described herein and/or the CAR polypeptides, and then administered to the same subject. In one specific embodiment, prior to re-introduction into the subject, the autologous immune cells (e.g., T or NK cells) are activated and/or expanded ex vivo. Administration of autologous cells to a subject may result in reduced rejection of the immune cells as compared to administration of non-autologous cells.

Alternatively, the immune cells are allogeneic cells, i.e., the cells are obtained from a first subject, genetically engineered for expression of at least two metabolism modulating polypeptides described herein and/or the CAR polypeptide and administered to a second subject that is different from the first subject but of the same species. For example, allogeneic immune cells may be derived from a human donor and administered to a human recipient who is different from the donor. In a specific embodiment, the T cells are allogeneic T cells in which the expression of the endogenous T cell receptor has been inhibited or eliminated. In one specific embodiment, prior to introduction into the subject, the allogeneic T cells are activated and/or expanded ex vivo. T lymphocytes can be activated by any method known in the art, e.g., in the presence of anti-CD3/CD28, IL-2, IL-15, phytohemoagglutinin, engineered artificial stimulatory cells or particles, or a combination thereof.

NK and T cells (αβ T or γδ T cells) can be activated by any method known in the art, e.g., in the presence of one or more agents selected from the group consisting of CD137 ligand protein, CD137 antibody, IL-15, IL-15 receptor antibody, IL-2, IL-12, IL-21, and cells from the K562 cell line, and/or engineered artificial stimulatory cells or particles. See, e.g., U.S. Pat. Nos. 7,435,596 and 8,026,097 for the description of useful methods for expanding NK cells. For example, NK cells used in the compositions or methods of the disclosure may be preferentially expanded by exposure to cells that lack or poorly express major histocompatibility complex I and/or II molecules and which have been genetically modified to express membrane bound IL-15 and 4-1BB ligand (CD137L). Such cell lines include, but are not necessarily limited to, K562 [ATCC, CCL 243; (Lozzio and Lozzio, Blood, 45(3): 321-334 (1975); Klein et al., Int J Cancer, 18(4): 421-431 (1976))], and the Wilms tumor cell line HFWT (Fehniger and Caligiuri, Int Rev Immunol, 20(3-4): 503-534 (2001); Harada et al., Exp Hematol, 32(7): 614-621 (2004)), the uterine endometrium tumor cell line HHUA, the melanoma cell line HMV-II, the hepatoblastoma cell line HuH-6, the lung small cell carcinoma cell lines Lu-130 and Lu-134-A, the neuroblastoma cell lines NB19 and N1369, the embryonal carcinoma cell line from testis NEC 14, the cervix carcinoma cell line TCO-2, and the bone marrow-metastasized neuroblastoma cell line TNB 1 (Harada et al., Jpn J Cancer Res, 93(3): 313-319 (2002)). Preferably the cell line used lacks or poorly expresses both MHC I and II molecules, such as the K562 and HFWT cell lines. A solid support may be used instead of a cell line. Such support should preferably have attached on its surface at least one molecule capable of binding to NK cells and inducing a primary activation event and/or a proliferative response or capable of binding a molecule having such an affect thereby acting as a scaffold. The support may have attached to its surface the CD137 ligand protein, a CD137 antibody, the IL-15 protein or an IL-15 receptor antibody. Preferably, the support will have IL-15 receptor antibody and CD137 antibody bound on its surface.

In one embodiment of the described compositions or methods, introduction (or reintroduction) of T lymphocytes, NK cells, or T lymphocytes and NK cells to the subject is followed by administering to the subject a therapeutically effective amount of IL-2.

In additional aspects, the method further comprises cryopreserving the population of engineered NK or T cells. In some instances, the engineered NK, αβ T cells or γδ T cells are cryopreserved. Further provided herein is a genetically engineered population of cryopreserved NK or T cells.

In accordance with the present disclosure, patients can be treated by infusing therapeutically effective doses of immune cells such as T or NK cells comprising at least two metabolism modulating polypeptides and/or a CAR polypeptide of the disclosure in the range of about 105 to 1010 or more cells per kilogram of body weight (cells/Kg). The infusion can be repeated as often and as many times as the patient can tolerate until the desired response is achieved. The appropriate infusion dose and schedule will vary from patient to patient, but can be determined by the treating physician for a particular patient. Typically, initial doses of approximately 10⁶ cells/kg will be infused, escalating to 108 or more cells/kg. IL-2 can be co-administered to expand infused cells. The amount of IL-2 can be about 1-5×10⁶ international units per square meter of body surface.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The efficacy of the compositions or methods described herein may be assessed by any method known in the art and would be evident to a skilled medical professional. For example, the efficacy of the compositions or methods described herein may be assessed by survival of the subject or cancer or pathogen burden in the subject or tissue or sample thereof. In some embodiments, the compositions and methods described herein may be assessed based on the safety or toxicity of the therapy (e.g., administration of the immune cells expressing the metabolism modulating polypeptides that redirect glucose metabolites and the CAR polypeptides) in the subject, for example, by the overall health of the subject and/or the presence of adverse events or severe adverse events.

### C. Other Immunotherapies

In some embodiments, the genetically engineered immune cells of the present invention may be derived from natural immune cells specific to diseased cells (e.g., cancer cells or pathogen infected cells). Such genetically engineered immune cells (e.g., tumor-infiltrating lymphocytes or TILs) may not co-express any chimeric receptor polypeptide and can be used to destroy the target disease cells, e.g., cancer cells. The genetically engineered TILs, expressing at least two metabolism modulating polypeptides but not chimeric receptors, may be co-used with a bispecific antibody capable of binding to the target tumor cells and the TILs (e.g., mediated by an anti-CD3 binder as in bispecific T cell engagers “BiTE”, see (Zhou et al., Biomarker Research, 9(1): 38 (2021))). In some embodiments, the genetically engineered immune cells, of the present invention may be T_{reg} cells. Such T_{reg} cells may coexpress a chimeric receptor polypeptide as disclosed herein. Alternatively, the T_{reg} cells may not co-express any chimeric receptor polypeptide and can be used for the intended therapy.

Some embodiments provide for a composition comprising an effective amount of the engineered NK or T cells of the embodiments for use in the treatment of a disease or disorder in a subject. Also provided herein is the use of a composition comprising an effective amount of the engineered NK or T cells of the embodiments for the treatment of an immune-related disorder in a subject. A further embodiment provides for a method of treating an immune-related disorder in a subject comprising administering an effective amount of engineered NK or γδ T cells of the embodiments to the subject. In exemplary embodiments, the method does not comprise performing HLA matching. In particular embodiments, the NK or γδ T cells are KIR-ligand mismatched between the subject and donor. In further specific embodiments, the method does not comprise performing HLA matching. In particular embodiments, the absence of HLA matching does not result in graft versus host disease or toxicity.

### V. Combination Treatments

The compositions and methods described in the present disclosure may be utilized in conjunction with other types of therapy for cancer, such as chemotherapy, immunotherapy, surgery, radiation, gene therapy, and so forth, or anti-infection therapy. Such therapies can be administered simultaneously or sequentially (in any order) with the immunotherapy according to the present disclosure. When co-administered with an additional therapeutic agent, suitable therapeutically effective dosages for each agent may be lowered due to the additive action or synergy.

In some embodiments, the genetically engineered immune cells (e.g., T and/or NK cells) disclosed herein may be administered to a subject who has been treated or is being treated with an additional therapeutic agent (e.g., an additional anti-cancer therapeutic agent). For example, these genetically engineered immune cells may be administered to a human subject simultaneously with the additional therapeutic agent. In some embodiments, the genetically engineered immune cells may be administered to a human subject before the additional therapeutic agent. In some embodiments, the genetically engineered immune cells may be administered to a human subject after the additional therapeutic agent.

Genetically engineered immune cells (e.g., T cells or NK cells) that co-express at least two metabolism modulating polypeptides and a CAR polypeptide specific to a tag can be co-used with a therapeutic agent conjugated to the tag. Via the therapeutic agent, which is capable of binding to an antigen associated with diseased cells such as tumor cells, such genetically engineered immune cells can be engaged with the diseased cells and inhibit their growth. Any of the antibodies listed in Table 4 above, or others specific to the same target antigen also listed in Table 4 can be conjugated to a suitable tag (e.g., those described herein) and be co-used with immune cells co-expressing the metabolic modulating polypeptides and a CAR polypeptide specific to the tag.

The treatments of the disclosure can be combined with other immunomodulatory treatments such as, e.g., therapeutic vaccines (including but not limited to GVAX, dendritic cell (DC)-based vaccines, etc.), checkpoint inhibitors (including but not limited to agents that block CTLA-4, PD-1, LAG3, TIM3, etc.) or activators (including but not limited to agents that enhance 4-1BB, OX40, etc.). In some embodiments, genetically engineered immune cells (e.g., T cells or NK cells) that co-express at least two metabolism modulating polypeptides and a CAR polypeptide is combined with an immunomodulatory treatment.

Non-limiting examples of other therapeutic agents useful for combination with the immunotherapy of the disclosure include: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (Nature, 407(6801): 249-257 (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones, and navelbine, epidipodophyllotoxins (etoposide and teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamine oxaliplatin, iphosphamide, melphalan, merchlorehtamine, mitomycin, mitoxantrone, nitrosourea, plicamycin, procarbazine, paclitaxel, docetaxel, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycin, plicamycin (mithramycin) and mitomycin; enzymes (Lasparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (brefeldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); AKT inhibitors (such as MK-2206 2HCI, Perifosine (KRX-0401), GSK690693, Ipatasertib (GDC-0068), AZD5363, uprosertib, afuresertib, or triciribine); mTOR inhibitors, topoisomerase inhibitors (doxorubicin (adriamycin), amsacrine, camptothecin, daunorubicin, dactinomycin, eniposide, epirubicin, etoposide, idarubicin, mitoxantrone, topotecan, and irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors.

For examples of additional useful agents see also Physician's Desk Reference, 59^{th} edition, (2005), Thomson P D R, Montvale NJ; Gennaro et al., Eds. Remington's The Science and Practice of Pharmacy 20^{th} edition, (2000), Lippincott Williams and Wilkins, Baltimore Md.; Braunwald et al., Eds. Harrison's Principles of Internal Medicine, 15th edition, (2001), McGraw Hill, NY; Berkow et al., Eds. The Merck Manual of Diagnosis and Therapy, (1992), Merck Research Laboratories, Rahway N.J.

The administration of an additional therapeutic agent can be performed by any suitable route, including systemic administration as well as administration directly to the site of the disease (e.g., to a tumor).

In some embodiments, the method involves administering the additional therapeutic agent (e.g., an antibody) to the subject in one dose. In some embodiments, the method involves administering the additional therapeutic agent (e.g., an antibody) to the subject in multiple doses (e.g., at least 2, 3, 4, 5, 6, 7, or 8 doses). In some embodiments, the additional therapeutic agent (e.g., an antibody) is administered to the subject in multiple doses, with the first dose of the additional therapeutic agent (e.g., an antibody) administered to the subject about 1, 2, 3, 4, 5, 6, or 7 days prior to administration of the immune cells expressing at least two metabolism modulating polypeptides and/or the CAR polypeptide. In some embodiments, the first dose of the additional therapeutic agent (e.g., an antibody) is administered to the subject between about 24-48 hours prior to the administration of the immune cells described herein.

In some embodiments, the first dose of the additional therapeutic agent (e.g., an antibody) is administered to the subject prior to the administration of the genetically engineered immune cells described herein. In some embodiments, the additional therapeutic agent (e.g., an antibody) is administered to the subject prior to administration of the genetically engineered immune cells described herein and then subsequently about every two weeks. In some embodiments, the first two doses of the additional therapeutic agent (e.g., an antibody) are administered about one week (e.g., about 6, 7, 8, or 9 days) apart. In certain embodiments, the third and following doses are administered about every two weeks.

In any of the embodiments described herein, the timing of the administration of the additional therapeutic agent (e.g., an antibody) is approximate and includes three days prior to and three days following the indicated day (e.g., administration every three weeks encompasses administration on day 18, day 19, day 20, day 21, day 22, day 23, or day 24).

Efficacy of immune system induction for disease therapy may be enhanced by combination with other agents that, for example, reduce tumor burden prior to administration of the genetically engineered immune cells of the present invention. Antibody-drug conjugates (ADCs) can efficiently reduce tumor burden in many types of cancers. Numerous exemplary ADCs are known in the art (Mullard, Nat Rev Drug Discov, 12(5): 329-332 (2013); Coats et al., Clinical Cancer Research, 25(18): 5441-5448 (2019); Zhao et al., Acta Pharmaceutica Sinica B, 10(9): 1589-1600 (2020); Fu et al., Signal Transduction and Targeted Therapy, 7(1): 93 (2022)). Any such known ADC may be used in combination with a CAR-T or CAR-NK construct as described herein. Thus, in some embodiments, where an ADC is used in combination with a CAR-T or CAR-NK, the ADC is administered prior to the CAR-T or CAR-NK. In some embodiments, an ADC is used in combination with a CART or CAR-NK as disclosed herein. In some embodiments, the first dose of the ADC is administered to the subject prior to the administration of the genetically engineered immune cells described herein.

In another embodiment, the efficacy of the immune system induction for the disease therapy may be enhanced by combination with other immunotherapeutic agents, e.g., cytokines that stimulate the CAR-T or CAR-NK cells in vivo (e.g., agonists of the IL-2/IL15Rβγ such as IL-2, IL-15 (IL-2/IL-15 superagonists); IL-7, or IL-12, or derivatives thereof) or immune checkpoint inhibitors (e.g., anti-PD-1 antibodies, anti-PD-L1 antibodies, anti-LAG3 antibodies, anti-CTLA-4 antibodies or anti-TIM3 antibodies).

In some embodiments, the method further comprises administering a lymphocyte reduction treatment, preferably selected from cyclophosphamide and fludarabine. Such lymphodepletion treatment is preferably applied prior to the infusion of the immune cells expressing a CAR in order to allow for greater T cell expansion of the infused cells (Shank et al., Pharmacotherapy, 37(3): 334-345 (2017)).

The efficacy of the methods described herein may be assessed by any method known in the art and would be evident to a skilled medical professional and/or those described herein.

### VI. Kits for Therapeutic Use

The present disclosure also provides kits for use of any of the compositions described herein. For example, the present disclosure also provides kits comprising a population of genetically engineered immune cells (e.g., T or NK cells, constructed in vitro or in vivo) that express at least two metabolism modulating polypeptides and optionally a chimeric receptor (ACTR or CAR) polypeptide described herein for use in inhibiting the growth of diseased cells, e.g., tumor cells and/or enhancing immune cell growth and/or proliferation in a low glucose environment, a low amino acid environment, a low-pH environment, and/or hypoxic environment, for example, in a tumor microenvironment. The kit may further comprise a therapeutic agent or a therapeutic agent conjugated to a tag (e.g., those described herein), to which the chimeric receptor polypeptide expressed on the immune cells bind. Such kits may include one or more containers comprising the population of the genetically engineered immune cells as described herein (e.g., T and/or NK cells), and optionally a therapeutic agent or a therapeutic agent conjugated to a tag.

In some embodiments, the kit comprises genetically engineered immune cells described herein which are expanded ex vivo. In another embodiment, the kit comprises genetically engineered immune cells described herein and an antibody specific to a cell surface antibody that is present on activated T cells, for example, an anti-CD5 antibody, an anti-CD38 antibody or an anti-CD7 antibody. In exemplary embodiments, the kit comprises NK or T cells expressing at least the two metabolism modulating polypeptides and CAR constructs known in the art or disclosed herein.

Alternatively, the kit disclosed herein may comprise a nucleic acid or a nucleic acid set as described herein, which collectively encodes any of the chimeric receptor polypeptides and at least the two metabolism modulating polypeptides as also described herein.

In some embodiments, the kit can additionally comprise instructions for use in any of the methods described herein. The included instructions may comprise a description of administration of the first and second pharmaceutical compositions to a subject to achieve the intended activity, e.g., inhibiting target cell growth in a subject. The kit may further comprise a description of selecting a subject suitable for treatment based on identifying whether the subject is in need of the treatment. In some embodiments, the kit may further comprise a description of selecting a subject suitable for treatment based on identifying whether the subject is in need of the treatment. Non-limiting examples of such methods of identification may include expression of target in blood, DNA or tissue (e.g., immunohistochemistry). Further, in some instances, a cut-off range may be used to adjust treatment dosage.

In some embodiments, the instructions comprise a description of administering the population of genetically engineered immune cells (T or NK cells) and optionally a description of administering the tag-conjugated therapeutic agent. The instructions relating to the use of the immune cells (T or NK cells) and optionally the tag-conjugated therapeutic agent as described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the second pharmaceutical composition is an antibody as described herein. At least one active agent in the first pharmaceutical composition is a population of genetically engineered immune cells as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### GENERAL TECHNIQUES

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985); Transcription and Translation (B. D. Hames & S. J. Higgins, eds. (1984»; Animal Cell Culture (R. I. Freshney, ed. (1986); Immobilized Cells and Enzymes (IRL Press, (1986); and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are intended only to illustrate methods and embodiments in accordance with the invention, and as such should not be construed as imposing limitations upon the claims.

### Example 1: Impact of Expressing at Least Two Polypeptides on Immune Cell Function Expressing an ACTR Polypeptide in Lower Glucose Environments

At least two transgenes encoding at least two metabolism modulating polypeptides (e.g., any two of SEQ ID NO: 75-SEQ ID NO: 82) are co-expressed in the same T and/or NK cell with an ACTR polypeptide. The transgenes are, for example, encoding GOT2 and TIGAR (e.g., SEQ ID NO: 77 and SEQ ID NO: 75). The T and/or NK cells are transduced with a virus encoding the ACTR polypeptide and at least two metabolic modulating polypeptides described herein (SEQ ID NO: 75-SEQ ID NO: 82) separated, for example, by a P2A ribosomal skip sequence. The T and/or NK cells are mixed at a given effector-to-target (E:T) ratio with tumor target cells, such as IGROV-1 cells, and a tumor-targeting antibody such as an anti-FOLRα antibody. Reactions are then incubated at 37° C. in a 5% CO₂ incubator for a period of time (e.g., 6-8 days) at different starting concentrations of glucose (e.g., 0-20 mM). T and/or NK cell function is then evaluated, for example, using cytokine production or proliferation assays or for resistance to chronic stimulation. Cytokine production (e.g., IL-2 and/or IFN-γ) is measured from the reaction supernatant. For proliferation experiments, co-cultures are harvested and stained with α-CD3, α-CD14, α-CD33, α-CD45, α-CD56 antibodies and a live-dead cell stain. As a measure of T cell proliferation, the live T cells is enumerated in CD45⁺CD33⁻CD3⁺CD14⁻CD56⁻ cells and a live-dead cell stain is evaluated by flow cytometry. And in case of NK cells, enumeration is carried on CD45⁺CD33⁻CD3⁻CD14⁻CD56⁺ and a live-dead cell stain is evaluated by flow cytometry.

T and/or NK cells expressing at least two polypeptides described herein in addition to the ACTR polypeptide show enhanced T and/or NK cell function relative to T and/or NK cells expressing ACTR alone including, for example, enhanced cytokine production or enhanced proliferation. This enhanced function may be more pronounced at lower glucose concentrations. These experiments demonstrate that expressing at least two such polypeptides in immune cells (such as T and/or NK) has a positive impact on immune cell activity.

### Example 2: Impact of Expressing at Least Two Polypeptides on Immune Cell Function Expressing an ACTR Polypeptide in Environments with Higher Soluble Inhibitor Concentrations

At least two transgenes encoding at least two metabolism modulating polypeptides (e.g., any two of SEQ ID NO: 75-SEQ ID NO: 82) are co-expressed in the same T and/or NK cell with an ACTR polypeptide. The transgenes are, for example, encoding GOT2 and TIGAR (e.g., SEQ ID NO: 77 and SEQ ID NO: 75). The T and/or NK cells are transduced with a virus encoding the ACTR polypeptide and at least two metabolic modulating polypeptides described herein (SEQ ID NO: 75-SEQ ID NO: 82) separated, for example, by a P2A ribosomal skip sequence. The T and/or NK cells are mixed at a given effector-to-target (E:T) ratio with tumor target cells, such as IGROV-1 cells, and a tumor-targeting antibody such as an anti-FOLRα antibody in media containing different concentrations of soluble inhibitors that are present in the tumor microenvironment (e.g., TGFB, PGE2, kynurenine, and/or adenosine). Reactions are then incubated at 37° C. in a 5% CO₂ incubator for a period of time (e.g., 6-8 days). NK and/or T cell function is then evaluated, for example, using cytokine production or proliferation assays or for resistance to chronic stimulation. Cytokine production (e.g., IL-2 and/or IFN-γ) is measured from the reaction supernatant. For proliferation experiments, co-cultures of NK and/or T cells are harvested, stained and evaluated by flow cytometry (see Example 1).

T and/or NK cells expressing at least two polypeptides described herein in addition to the ACTR polypeptide show enhanced cellular function relative to NK and/or T cells expressing ACTR alone including, for example, enhanced cytokine production. This enhanced function may be achieved at higher soluble inhibitor concentrations. These experiments demonstrate that expressing at least two such polypeptides in immune cells (such as T and/or NK) has a positive impact on immune cell activity.

### Example 3: Impact of Expressing at Least Two Polypeptides on Immune Cell Function Expressing an ACTR Polypeptide in Environments with Greater Immunosuppressive Cell Presence

At least two transgenes encoding at least two metabolism modulating polypeptides (e.g., any two of SEQ ID NO: 75-SEQ ID NO: 82) are co-expressed in the same T and/or NK cell with an ACTR polypeptide. The transgenes are, for example, encoding GOT2 and TIGAR (e.g., SEQ ID NO: 77 and SEQ ID NO: 75). The T and/or NK cells are transduced with a virus encoding the ACTR polypeptide and at least two metabolic modulating polypeptides described herein (SEQ ID NO: 75-SEQ ID NO: 82) separated, for example, by a P2A ribosomal skip sequence. The T and/or NK cells are mixed at a given effector-to-target (E:T) ratio with tumor target cells, such as IGROV-1 cells, and a tumor-targeting antibody such as an anti-FOLRα antibody, in the presence of immunosuppressive cells (e.g., myeloid-derived suppressor cells and/or regulatory T cells). Reactions are then incubated at 37° C. in a 5% CO₂ incubator for a period of time (e.g., 3-10 days). Immune cell (NK and/or T cell) function is then evaluated, for example, using cytokine production or cell proliferation assays or for resistance to chronic stimulation. Cytokine production (e.g., IL-2 and/or IFN-γ) is measured from the reaction supernatant. Proliferation experiments are performed and evaluated as described in Example 1.

T and/or NK cells expressing at least two polypeptides described herein in addition to the ACTR or CAR polypeptide show enhanced T and/or NK cell function relative to T and/or NK cells expressing ACTR or CAR alone including, for example, enhanced cytokine production or enhanced proliferation. This enhanced function may be achieved in the presence of increased amounts (e.g., greater number or percentage) of immunosuppressive cells. These experiments demonstrate that expressing at least two such polypeptides in immune cells (such as T and/or NK) has a positive impact on immune cell activity.

### Example 4: Impact of Expressing at Least Two Exemplary Polypeptides on Immune Cell Function Expressing an ACTR Polypeptide on Tumor Models

At least two transgenes encoding at least two metabolism modulating polypeptides (e.g., any two of SEQ ID NO: 75-SEQ ID NO: 82) are co-expressed in the same T and/or NK cell with an ACTR polypeptide. The transgenes are, for example, encoding GOT2 and TIGAR (e.g., SEQ ID NO: 77 and SEQ ID NO: 75). The T and/or NK cells are transduced with a virus encoding the ACTR polypeptide and at least two metabolic modulating polypeptides described herein (SEQ ID NO: 75-SEQ ID NO: 82) separated, for example, by a P2A ribosomal skip sequence. The T and/or NK cells are mixed at a given effector-to-target (E:T) ratio with tumor target cells, such as IGROV-1 cells, and a tumor-targeting antibody such as an anti-FOLRα antibody, is inoculated into NSG™ (NOD scid gamma, NOD.Cg-Prkdc_{scid} IL2rg^{tm1Wi1}/SzJ. Strain 005557) mice. Tumor-bearing mice are subsequently dosed with a tumor-targeting antibody, for example an anti-FOLRα antibody, and T and/or NK cells expressing ACTR alone or ACTR and metabolism modulating polypeptides. Tumor growth is monitored throughout the course of the experiment.

In combination with a tumor-targeting antibody, T and/or NK cells expressing at least two polypeptides described herein in addition to an ACTR polypeptide show enhanced antitumor activity relative to T and/or NK cells expressing an ACTR polypeptide alone. Additionally, in combination with a tumor-targeting antibody, T and/or NK cells expressing at least two polypeptides described herein in addition to an ACTR polypeptide may show enhanced T and/or NK cell activity including, for example, enhanced proliferation, enhanced T and/or NK cell persistence, and/or enhanced cytokine production relative to T and/or NK cells expressing the ACTR polypeptide alone. These experiments demonstrate that expressing at least two such polypeptides in ACTR-expressing immune (such as T and/or NK) cells has a positive impact on immune cell function in vivo.

### Example 5: Impact of Reduced Glucose Concentrations on Immune Cell Function

Gamma-retrovirus encoding an exemplary GPC3-targeting CAR expression construct (SEQ ID NO: 86 or SEQ ID NO: 87) was generated via recombinant technology and used to infect primary human T-cells for generating cells that express a GPC3-targeting CAR polypeptide on their cell surface. A six-day flow-based proliferation assay was then used to test the functionality of the GPC3-targeting CAR expressing cells. Specifically, 200,000 untransduced mock T-cells or T-cells expressing the GPC3-targeting CAR construct are incubated together at a ratio of 4:1 (effector cells/CAR-expressing T cells to target cells) with either 50,000 GPC3⁺ hepatocellular carcinoma JHH7 or Hep3B tumor cells. The co-culture is incubated at 37° C. in a 5% CO₂ incubator for six days in the presence of different concentrations of glucose. At the end of six days, co-cultures are harvested and stained with an anti-CD3 antibody. The number of CD3-positive cells was evaluated by flow cytometry as a measure of T cell proliferation. At lower glucose concentrations, less CAR-T proliferation is observed. These experiments demonstrate that low glucose environments may have a negative impact on CAR-T cell proliferation activity.

### Example 6: Impact of Expressing at Least Two Exemplary Polypeptides on Immune Cell Function Using a GPC3-Targeting CAR-T or CAR-NK Expression Construct

Gamma-retrovirus encoding an exemplary GPC3-targeting CAR polypeptide expression construct (e.g., any two of SEQ ID NO: 86 or SEQ ID NO: 87) is generated via recombinant technology and used to infect primary human T- or NK-cells to generate cells expressing a GPC3-targeting CAR polypeptide on their cell surface. In the constructs encoding both the CAR polypeptide and at least two factors, the three polypeptides are separated, for example, by at least one P2A ribosomal skip sequence. A six-day flow-based proliferation assay is then used to test the functionality of the GPC3-targeting CAR expressing cells. Specifically. 200,000 untransduced mock T and/or NK cells, T and/or NK cells expressing a GPC3-targeting CAR polypeptide, or T and/or NK cells expressing a GPC3-targeting CAR polypeptide and at least two metabolism modulating polypeptides are incubated together at a ratio of 4:1 (effector cells/CAR-expressing T and/or NK cells to target cells) with 50,000 GPC3⁺ hepatocellular carcinoma JHH7 tumor cells. The co-culture is incubated at 37° C. in a 5% CO₂ for six days in the presence of 1.25 mM glucose (tumor-relevant) and 10 mM glucose (approximate peripheral blood levels). At the end of six days, co-cultures are harvested, and co-cultures are harvested and stained with α-CD3, α-CD14, α-CD33, α-CD45, α-CD56 antibodies and a live-dead cell stain. As a measure of T cell proliferation, the live T cells is enumerated in CD45⁺CD33⁻CD3⁺CD14⁻CD56⁻ cells and a live-dead cell stain is evaluated by flow cytometry. And in case of NK cells, enumeration is carried on CD45⁺CD33⁻CD3⁻CD14⁻CD56⁺ and a live-dead cell stain is evaluated by flow cytometry.

Immune cells expressing the at least the two polypeptides described herein in addition to the CAR polypeptide demonstrate enhanced T and/or NK cell proliferation relative to T and/or NK cells expressing the CAR construct alone. This enhanced proliferation also occurs at tumor-relevant low glucose concentrations. These experiments demonstrate that expressing at least two factors in immune (such as T or NK) cells has a positive impact on CAR-T and/or CAR-NK cell proliferation activity.

### Example 7: Impact of Expressing at Least Two Exemplary Polypeptides on Immune Cell Function Expressing a CAR Polypeptide in Environments with Higher Soluble Inhibitor Concentrations

At least two transgenes encoding at least two metabolism modulating polypeptides (e.g., any two of SEQ ID NO: 75-SEQ ID NO: 82) are co-expressed in the same T and/or NK cells with a CAR polypeptide. The transgenes are, for example, encoding GOT2 and TIGAR (e.g., SEQ ID NO: 77 and SEQ ID NO: 75). The T and/or NK cells are transduced with a virus encoding the CAR polypeptide and the at least two polypeptides described herein (SEQ ID NO: 75-SEQ ID NO: 82) separated, for example, by a P2A ribosomal skip sequence. Transduced T and/or NK cells are mixed at a given effector-to-target (E:T) ratio with tumor target cells, such as HepG2 cells, in media containing different concentrations of soluble inhibitors that are present in the tumor microenvironment (e.g., TGFB, PGE2, kynurenine, and/or adenosine). Reactions are then incubated at 37° C. in a 5% CO₂ incubator for a period of time (e.g., 6-8 days). NK and/or T cell function is then evaluated, for example, using cytokine production or proliferation assays or for resistance to chronic stimulation. Cytokine production (e.g., IL-2 and/or IFN-γ) is measured from the reaction supernatant. For proliferation experiments, co-cultures of NK and/or T cells are harvested, stained and evaluated by flow cytometry.

T and/or NK cells expressing at least two polypeptides described herein in addition to the CAR polypeptide show enhanced T and/or NK cell function relative to T or NK cells expressing CAR alone including, for example, enhanced cytokine production or enhanced proliferation. This enhanced function may be achieved at higher soluble inhibitor concentrations. These experiments demonstrate that expressing at least two polypeptides described herein in immune (such as T or NK) cells has a positive impact on immune cell activity.

### Example 8: Impact of Expressing at Least Two Exemplary Polypeptides on Immune Cell Function Expressing a CAR Polypeptide in Environments with Greater Immunosuppressive Cell Presence

At least two transgenes encoding at least two metabolism modulating polypeptides (e.g., any two of SEQ ID NO: 75-SEQ ID NO: 82) are co-expressed in the same T and/or NK cells with a CAR polypeptide. The transgenes are, for example, encoding GOT2 & TIGAR (e.g., SEQ ID NO: 77 and SEQ ID NO: 75). The T and/or NK cells are transduced with a virus encoding the CAR polypeptide and the at least two polypeptides described herein (SEQ ID NO: 75-SEQ ID NO: 82) separated, for example, by a P2A ribosomal skip sequence. Transduced T and/or NK cells are mixed at a given effector-to-target (E:T) ratio with tumor target cells, such as HepG2 cells, in the presence of immunosuppressive cells (e.g., myeloid-derived suppressor cells and/or regulatory T cells). Reactions are then incubated at 37° C. in a 5% CO₂ incubator for a period of time (e.g., 3-10 days). T and/or NK cell function is then evaluated, for example, using cytokine production or cell proliferation assays or for resistance to chronic stimulation. Cytokine production (e.g., IL-2 and/or IFN-?) is measured from the reaction supernatant. Proliferation experiments is performed and evaluated as described in Example 1.

T and/or NK cells expressing the at least two polypeptides described herein in addition to the CAR polypeptide show enhanced T and/or NK cell function relative to T and/or NK cells expressing CAR alone including, for example, enhanced cytokine production or enhanced proliferation. This enhanced function may be achieved in the presence of increased amounts (e.g., greater number or percentage) of immunosuppressive cells. These experiments demonstrate that expressing at least two such polypeptides in immune (such as T and/or NK) cells has a positive impact on immune cell activity.

### Example 9: Impact of Expressing at Least Two Exemplary Polypeptides on T Cell Function Expressing a CAR Polypeptide in Tumor Models

At least two transgenes encoding at least two metabolism modulating polypeptides (e.g., any two of SEQ ID NO: 75-SEQ ID NO: 82) are co-expressed in the same T and/or NK cells with a CAR polypeptide. The transgenes are, for example, encoding GOT2 & TIGAR (e.g., SEQ ID NO: 77 and SEQ ID NO: 75). The T and/or NK cells are transduced with a virus encoding the CAR polypeptide and the at least two polypeptides described herein (SEQ ID NO: 75-SEQ ID NO: 82) separated, for example, by a P2A ribosomal skip sequence. Transduced T and/or NK cells are evaluated for anti-tumor activity in mouse tumor models. For these experiments, a tumor cell line, for example HepG2, is inoculated into NSG™ (NOD scid gamma, NOD.Cg-Prkdc^{scid} IL2rg^{tmWj1}/SzJ, Strain 005557) mice. Tumor-bearing mice are subsequently dosed with T and/or NK cells expressing CAR alone or CAR and metabolism modulating polypeptides. Tumor growth is monitored throughout the course of the experiment.

NK and/or T cells expressing the metabolism modulating polypeptides described herein in addition to a CAR polypeptide show enhanced anti-tumor activity relative to T and/or NK cells expressing a CAR polypeptide alone. Additionally, T and/or NK cells expressing at least two polypeptides described herein in addition to a CAR polypeptide may show enhanced T and/or NK cell activity including, for example, enhanced proliferation, persistence, and/or cytokine production relative to T and/or NK cells expressing the CAR polypeptide alone. These experiments demonstrate that expressing the at least two such polypeptides in CAR-expressing T and or NK cells has a positive impact on T and/or NK cell function in vivo.

### Example 10: Expression of GLUT1, GOT2, and TIGAR Elevated Glucose Uptake and Lactate Production

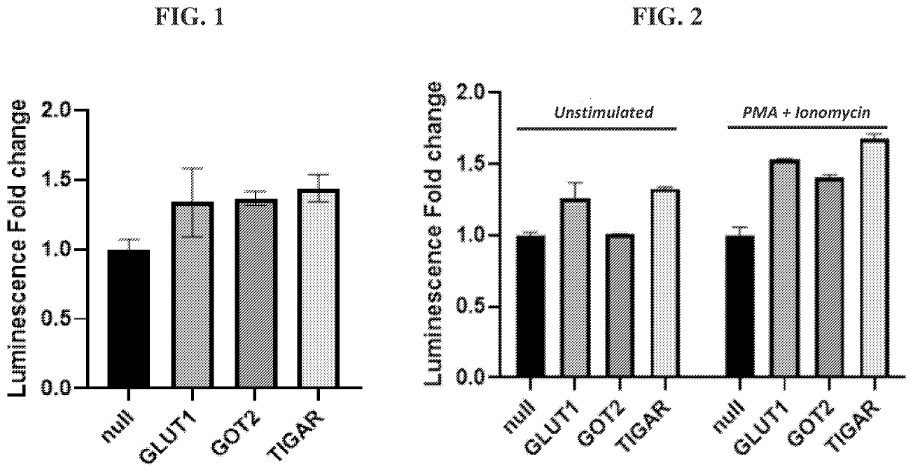

Healthy donor PBMCs were stimulated with anti-CD3 and anti-CD28 until day 2 followed by transduction with V5-tagged transgene packaged into a lentiviral vector. The transgene encoded GLUT1 (SEQ ID NO: 76), GOT2 (SEQ ID NO: 77), or TIGAR (SEQ ID NO: 75). The transduced cells were supplemented with fresh IL-2 each day until day 10. 10,000 cells/well (384-well plate) were resuspended in PBS and assayed for glucose uptake. The luminescence read-out was evaluated as a fold change for each transgene were compared to null (non-transduced control; baseline as fold change 1) T cells under the same condition. Cells transduced with GLUT1, GOT2 or TIGAR showed elevated levels of glucose uptake, which is indicative of higher metabolic activity. Data are representative of three donors. See FIG. 1.

Further, healthy donor PBMCs were stimulated with anti-CD3 and anti-CD28 until day 2 followed by transduction with V5-tagged transgene (described above) packaged into lentiviral vectors. The transduced cells were supplemented with fresh IL-2 each day until day 9. On day 9, a subset of T cells was stimulated with phorbol myristate acetate (PMA) and Ionomycin for 24 h. 10,000 harvested cells/well (384-well plate) were resuspended in RPMI without FBS and incubated at 37° C. for 2 h to remove residual lactate from the media and assayed for lactate production. The luminescence read-out was evaluated as a fold change for each transgene and were compared to null (non-transduced control; baseline as fold change 1) T cells under the same condition. Stimulated cells transduced with GLUT1, GOT2 and TIGAR showed elevated levels of lactate production indicative of higher metabolic adaptability in nutrient deficient environments. See FIG. 2. Data are representative of three donors.

In sum, the results of this example show that T cells transduced with GLUT1, GOT2, or TIGAR showed enhanced metabolic activity and accordingly adaptability in nutrient deficient environment. TIGAR showed the best effects among the three, especially under stimulated conditions simulating the stimulated condition of TILs in the tumor. This indicates that therapeutic T cells (e.g., T cells expressing an ACTR or CAR polypeptide as disclosed herein) co-expressing the metabolism modulating polypeptides GLUT1, GOT2, or TIGAR (specifically TIGAR) would be better adapted to tumor microenvironment (which could be deficient in nutrient) and exhibit better therapeutic activity as compared with counterpart T cells that are not transduced with the GLUT1, GOT2, or TIGAR transgene. See also WO2020/010110 and WO2020/037066, the relevant disclosures of each of which are incorporated by reference for the subject matter and purpose referenced herein.

### Example 11: Impact of Expressing at Least Two Exemplary Polypeptides in Immune Cells Expressing an ACTR Polypeptide

At least two transgenes encoding at least two metabolism modulating polypeptides (e.g., any two of SEQ ID NO: 75-SEQ ID NO: 82) are co-expressed in the same T and/or NK cell with an ACTR polypeptide. The transgenes are, for example, encoding GOT2 and TIGAR (SEQ ID NO: 77 and SEQ ID NO: 75). The T and/or NK cells are transduced with a virus encoding the ACTR polypeptide and at least two metabolic modulating polypeptides described herein (SEQ ID NO: 75-SEQ ID NO: 82) separated, for example, by a P2A ribosomal skip sequence. The T and/or NK cells are mixed at a given effector-to-target (E:T) ratio with tumor target cells, such as IGROV-1 cells, and a tumor-targeting antibody such as an anti-FOLRα antibody. The transduced cells are supplemented with cytokines (e.g., IL-2) for 3-10 days. All reactions are incubated at 37° C. in a 5% CO₂ incubator. For glucose uptake measurements, cells are harvested and assayed for glucose uptake using Glucose Uptake Glo Kit. This luminescence-based assay is evaluated and data represented as a fold change. Complimentary cell metabolic flux assays are performed to capture changes in basal oxygen consumption rate (OCR) using seahorse extracellular flux analyzer.

T and/or NK cells expressing the at least two metabolism modulating polypeptides described herein, in addition to the ACTR polypeptide, are expected to show enhanced glucose uptake. This enhanced function is suggestive of increased metabolic fitness and has a positive impact on the immune cell activity.

### Example 12: Impact of Expressing at Least Two Exemplary Polypeptides in Immune Cells Expressing a CAR Polypeptide

At least two transgenes encoding at least two metabolism modulating polypeptides (e.g., any two of SEQ ID NO: 75-SEQ ID NO: 82) are co-expressed in the same T and/or NK cells with a CAR polypeptide. The transgenes are, for example, encoding GOT2 and TIGAR (e.g., SEQ ID NO: 77 and SEQ ID NO: 75). The T and/or NK cells are transduced with a virus encoding the CAR polypeptide and the at least two polypeptides described herein (SEQ ID NO: 75-SEQ ID NO: 82) separated, for example, by a P2A ribosomal skip sequence. The transduced cells are supplemented with cytokines (e.g., IL-2) for 3-10 days. All reactions are incubated at 37° C. in a 5% CO₂ incubator. For glucose uptake measurements, cells are harvested and assayed for glucose uptake using Glucose Uptake Glo Kit. This luminescence-based assay is evaluated and data represented as a fold change. Complimentary cell metabolic flux assays are performed to capture changes in basal oxygen consumption rate (OCR) using seahorse extracellular flux analyzer.

T and/or NK cells expressing the at least two polypeptides described herein, in addition to the CAR polypeptide, are expected to show enhanced glucose uptake. This enhanced function is suggestive of increased metabolic fitness and has a positive impact on immune cell activity.

### Example 13: Impact of Expressing at Least Two Exemplary Polypeptides that Redirects Lactate Production in Immune Cells Expressing an ACTR Polypeptide

At least two transgenes encoding at least two metabolism modulating polypeptides (e.g., any two of SEQ ID NO: 75-SEQ ID NO: 82) are co-expressed in the same T and/or NK cell with an ACTR polypeptide. Exemplary ACTR polypeptides are disclosed in Table 10. The transgenes are, for example, encoding GOT2 and TIGAR (e.g., SEQ ID NO: 77 and SEQ ID NO: 75). The T and/or NK cells are transduced with a virus encoding the ACTR polypeptide and at least two metabolic modulating polypeptides described herein (SEQ ID NO: 75-SEQ ID NO: 82) separated, for example, by a P2A ribosomal skip sequence. The T and/or NK cells are mixed at a given effector-to-target (E:T) ratio with tumor target cells, such as IGROV-1 cells, and a tumor-targeting antibody such as an anti-FOLRα antibody. The transduced cells are supplemented with cytokines (e.g., IL-2) and additionally with stimulants (e.g., PMA and/or Ionomycin) for 3-10 days. All reactions are incubated at 37° C. in a 5% CO₂ incubator. Cells are harvested and assayed for lactate production using Lactate Glow Assay. This luminescence-based assay was evaluated, and data represented as a fold change.

T and/or NK cells expressing the at least two polypeptides described herein in addition to the ACTR polypeptide are expected to show enhanced lactate production. This enhanced function is suggestive of increased metabolic fitness and has a positive impact on immune cell activity.

### Example 14: Impact of Expressing at Least Two Exemplary Polypeptides that Redirects Lactate Production in Immune Cells Expressing a CAR Polypeptide

At least two transgenes encoding at least two metabolism modulating polypeptides (e.g., any two of SEQ ID NO: 75-SEQ ID NO: 82) are co-expressed in the same T and/or NK cells with a CAR polypeptide. The transgenes are, for example, encoding GOT2 & TIGAR (e.g., SEQ ID NO: 77 and SEQ ID NO: 75). The T cells are stimulated with anti-CD3 and anti-CD28 for a time period (e.g., 1-4 days) followed by transduction with virus (e.g., lentiviral or gamma-retroviral) encoding the CAR polypeptide and the at least two polypeptides described herein (SEQ ID NO: 75-SEQ ID NO: 82) separated, for example, by a P2A ribosomal skip sequence. The transduced cells are supplemented with cytokines (e.g., IL-2) and additionally with stimulants (e.g., PMA and/or Ionomycin) for 3-10 days. All reactions are incubated at 37° C. in a 5% CO₂ incubator. Cells are harvested and assayed for lactate production using Lactate Glow Assay. This luminescence-based assay was evaluated, and data represented as a fold change.

T and/or NK cells expressing the at least two polypeptides described herein, in addition to the CAR polypeptide, are expected to show enhanced lactate production. This enhanced function is suggestive of increased metabolic fitness and has a positive impact on immune cell activity.

### Example 15: Production of Retroviral Particles

On day 1, 12×10⁶ low passage HEK293T cells were plated on 15 cm coated tissue culture plates in DMEM media containing 10% FBS. The following day, i.e., day 2, the cells were 80% confluent. On day 3, the cells were subjected to transfection. 3 ml of transfection mix containing 10 μg of GAG/Pol, 6.6 ug of GALV helper, 20 μg of transfer plasmids and 74 μl of PEI Pro transfection reagent (Cat #115-010, PolyPlus) were prepared and added to the cell culture plates. The transfected cells were replenished with fresh DMEM media containing 10% FBS media 6 h post-transfection. The viral supernatants were harvested at 24 h and 36 h post-transfection and concentrated through a 0.45 μm filter and stored at −80° C. until further use.

### Example 16: Initiation and Transduction of Immune Cells

Immune cells (such as NK and/or T cells) were isolated either from fresh blood samples or are derived from cell lines.

Peripheral blood mononuclear cells (PBMCs) containing the immune cells were isolated by the density gradient method using Ficoll-paque. Briefly, equal volume of whole blood and PBS was mixed carefully by inversion, overlayed on Ficoll-paque followed by centrifugation at 400 g for 30 min at RT. The PBMCs were retrieved from the buffy layer (see (Low and Wan Abas, Biomed Res Int, 2015:239362 (2015)). PBMCs were stimulated with anti-CD3 and anti-CD28 antibodies until day 2 prior to transduction.

Approximately 100×10⁶ PBMCs from three donors were initiated by stimulation with anti-CD3 antibody (Miltenyi: 130-093-387), anti-CD28 antibody (Cat #130-093-375; Miltenyi Biotec), and IL-2 (100 UI/ml, Cat #0078-0495-61; Prometheus) in X-Vivo 15 (Cat #BEBP04-744Q) in 100 ml on Day 0 in T175 flasks. On Day 2, activated PBMCs were harvested and 1 ml containing 1×10⁶ cells were plated in a 24-well plate. For transduction, 1 ml of viral supernatant was added to each well. Plates were centrifuged at 1200 g for 45 min and allowed to rest overnight in an incubator at 37° C. One day after transduction, cells were transferred to a GREX vessel (Cat #80192M; Wilson Wolf) filled with X-VIVO+100 IU/ml IL-2. The cells were maintained for an additional week by supplementing IL-2 every 48 h. On Day 10, CAR-T cells were harvested and frozen for further analysis.

The NK-92 cell line was used in assessment of NK cell functions. 1×10⁶ NK-92 cells were grown in T75 flasks and stimulated with IL-2 (100 UI/ml) in RPMI media containing 10% FBS. The cells were maintained for one week by supplementing IL-2 (100 UI/ml) every 48 h.

At least two transgenes encoding at least two metabolism modulating polypeptides as disclosed herein (e.g., any two of SEQ ID NO: 75-SEQ ID NO: 82) are co-expressed in the same T and/or NK cell with an ACTR (see Table 10) or CAR (see Table 11 to Table 13) polypeptide. The transgenes are, for example, GOT2 and TIGAR (e.g., SEQ ID NO: 77 and SEQ ID NO: 75). The T and/or NK cells are transduced with a virus encoding the ACTR or CAR polypeptide and at least two polypeptides described herein (selected from SEQ ID NO: 75-SEQ ID NO: 82) separated, for example, by at least one P2A ribosomal skip sequence.

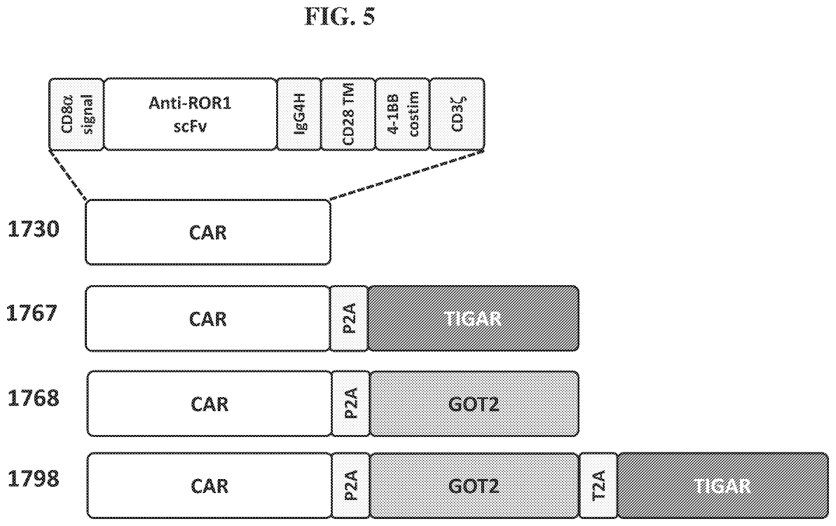

Recombinant antibodies targeting ROR1 were produced by a contract research organization. Briefly, antibody amino acids sequences for heavy and light chains were codon optimized for production with the constant domains from mouse or rabbit. Antibodies were produced using HEK293 transient expression and purified using Protein A chromatography and filtered through a 0.2 μm filter. A respective scFv was generated (SEQ ID NO: 93) and used to make a novel anti-ROR1 CAR (construct 1730; SEQ ID NO: 94) comprising the CD8α signal sequence, the anti-ROR1 scFv, the IgG4 hinge domain, the CD28 transmembrane domain, the 4-1BB co-stimulatory domain, and the CD33 cytoplasmic domain. The anti-ROR1 CAR was co-expressed with one or more transgenes. In case of one transgene (e.g., GOT2 as in case of construct 1768, SEQ ID NO: 96; see FIG. 5) was used, the anti-ROR1 CAR was separated from the single transgene by a single ribosomal skipping site P2A, or in case of two transgenes by two ribosomal skipping sites namely P2A and T2A (e.g., GOT2 and TIGAR as in case of clone 1798, SEQ ID NO: 97; see FIG. 5 and Table 17).

TABLE 17
anti-ROR1 CAR constructs co-expressing
metabolism modulating polypeptides

| | | | metabolism | | |
|---|---|---|---|---|---|
| | | Skipping | modulating | | SEQ |
| | scFv | site | polypeptides | Size | ID |
| 1730 | anti-ROR1 | none | none | 1.4 kbp | 94 |
| 1767 | anti-ROR1 | P2A | TIGAR | 2.2 kbp | 95 |
| 1768 | anti-ROR1 | P2A | GOT2 | 2.7 kbp | 96 |
| 1798 | anti-ROR1 | P2A + T2A | GOT2 + TIGAR | 3.6 kbp | 97 |

Briefly, 1×10⁶ cells were mixed with 1 ml of viral supernatant (see Example 1) in a total volume of 2 ml, centrifuged at 1200 g for 45 min followed by plating into a 24-well plate. The cells were then incubated at 37° C. in a 5% CO₂ incubator. In case of NK-92 transduced cells, the culture was monitored for growth every 48 h and split to a final concentration of 0.5×10⁶ cell/ml by supplementing IL-2 (100 UI/ml) every 48 h.

The transduced immune cells were assessed for the transgene expression by immunoblotting. The transduced cells (e.g., NK-92), were harvested by centrifuging at 1500 rpm for 5 min at RT. The supernatant was removed, and the cell pellet was washed twice in 1×PBS before flash freezing in liquid nitrogen and stored at −80° C. until further use. Cell pellets were subsequently lysed in 200 μl of SDS Lysis buffer (Cat #NP0008; Novex) containing 1x HALT Protease Inhibitor Cocktail (Cat #78430; Thermo Fisher Scientific) followed by sonication. The suspension was centrifuged at 15,000 rpm for 15 min at RT and the supernatant containing total protein was collected. The total protein concentration was measured using Pierce 660 nm Protein Assay (Cat #1861426; Thermo Fisher Scientific) followed by immunoblotting. 10 ug total protein was loaded in each lane of a Novex™ 4 to 12% Tris-Glycine Plus, 1.0 mm, 20-well Midi Protein Gel (Invitrogen), transferred onto PVDF membrane using Transblot Turbo (Biorad) and blocked for 1 h at RT using LICOR Blocking buffer. The membrane was probed for transgenes (e.g., GOT2, TIGAR) using mouse a-Actin (3700S, CST; dilution 1:2000), Rabbit a-TIGAR (14751S CST: dilution 1:1000) and Rabbit a-GOT2 (NBP232241, Novus; dilution 1:2000) antibodies overnight (in 0.1% Tween 20+LICOR Blocking buffer) at 4° C. The following day, membranes were washed thrice with 1x TBS containing 0.1% Tween20 detergent (w/v) for 5 min each. Membranes were subsequently incubated with standard rabbit or mouse secondary antibodies (LICOR; dilution 1:10,000) for 1 h. The membranes were washed thrice with 1x TBS containing 0.1% Tween20 detergent (w/v) for 5 min each. Immunoblots were imaged using a CLX imager (LICOR) and processed in the Image Studio Software (v5.2; LICOR).

### Example 17: Analysis of CAR Expression in Transduced Immune Cells

Immune cells (such as NK and/or T cells) were isolated either from fresh blood samples or are derived from cell lines and were transduced as described in Example 16. On day 7 post-transduction, the cells were harvested by centrifuging at 1500 rpm for 5 min at RT. The supernatant was removed, and the cell pellet was washed twice in 1×PBS followed by staining with Live Dead Aqua (Cat No. L34966; Thermo Fisher Scientific) for 10 min at RT.

In an example using the scFv having the sequence of SEQ ID NO: 93 targeted to ROR1, the CAR expression was evaluated by binding recombinant ROR1-Fc protein (Cat #RO1-H82F4, Acro Biosystems) followed by a streptavidin-PE secondary antibody (Cat #405204, BioLegend).

The cells were washed twice in 1x PBS followed by staining with primary and secondary antibody in 1×PBS with 2% FBS for assessing CAR expression. Living single cells were selected for and CAR expression was determined in comparison to an untransduced control (Null). Data were analyzed with FlowJo version 10.7.1 software (Tree Star Inc).

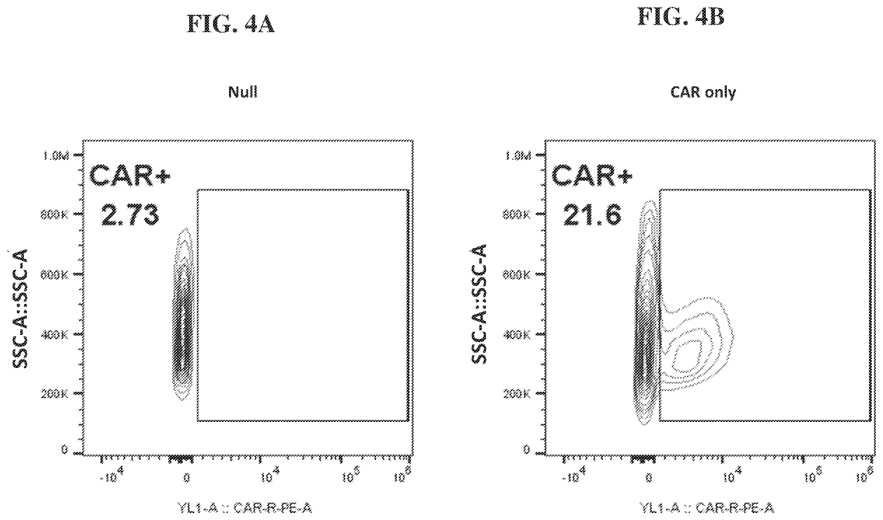

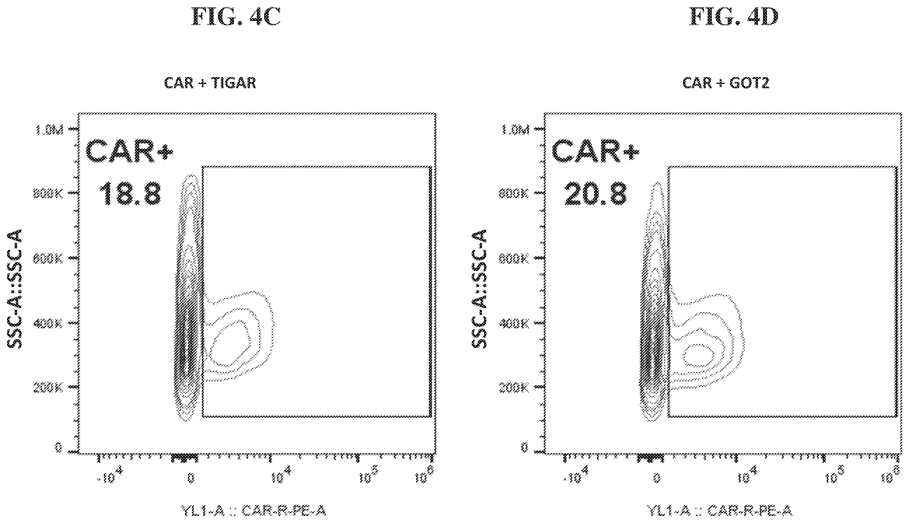

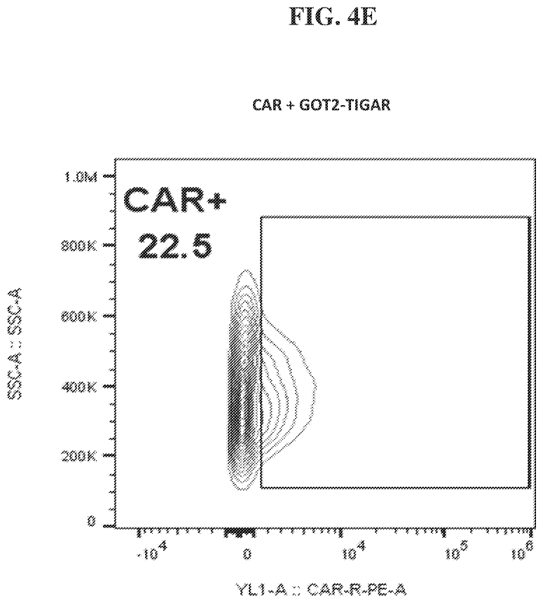

Co-expression of a CAR construct alone or together with TIGAR or GOT2 has been demonstrated in NK92 cells, an IL-2 dependent NK cell line derived from a patient with lymphoma. Transgene overexpression was analyzed for harvested cells on day 7 by immunoblotting as shown in FIG. 3. GOT2 expression was observed with all constructs and control due to the endogenous expression of GOT2, whereas a stronger band was observed in case of GOT2 co-expression with the CAR. No endogenous TIGAR expression was observed under these conditions. Strong expression was only seen once TIGAR was coexpressed with the CAR. In addition, harvested cells were stained with a recombinant antibody against the Fc part of the CAR, and presence of the CAR on the surface of the NK cell was assessed using flow cytometry. FIGS. 4A-4E show that all constructs expressed the CAR on the surface of the NK92 cells comparing to the null control (FIG. 4A).

### Example 18: Expression of at Least Two Exemplary Polypeptides in Immune Cells Expressing a CAR Polypeptide Enhances In Vitro Activity Under TME-Like Stress Conditions

Immune cells (such as NK and/or T cells) were isolated either from fresh blood samples (e.g., PBMCs) or derived from cell lines and were transduced as described in Example 16. CAR T (e.g., CAR only (1730), CAR and GOT2 (1768) or CAR, GOT2 and TIGAR (1798)) cells, alone or co-cultured with tumor cell lines either positive for ROR1 expression (e.g., endogenously such as A549 and CAKI-1 or engineered to over-express such as K562-hROR1) or negative for ROR1 expression (e.g., K562) were co-cultured for 24 h. The cytokine secretion (e.g., IFN-γ, IL-2, IL-6, IL-17, GM-CSF) and cytotoxicity were measured in response to a panel of cell lines either positive or negative for target expression (e.g., ROR1).

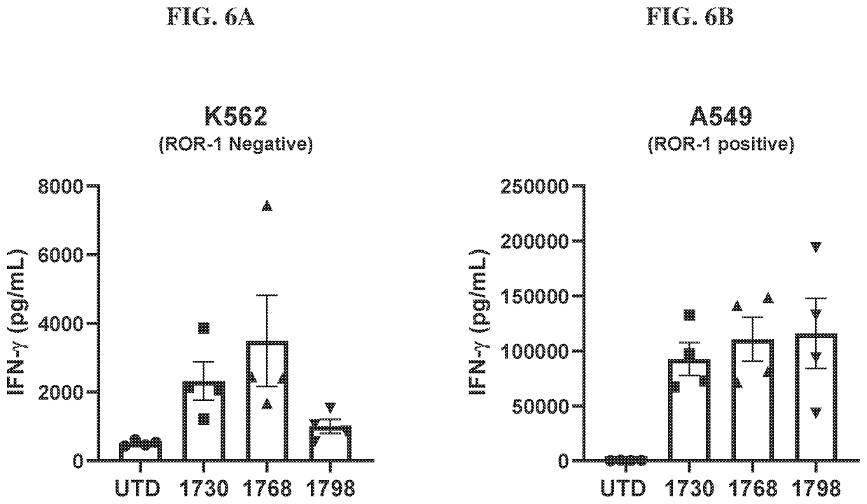

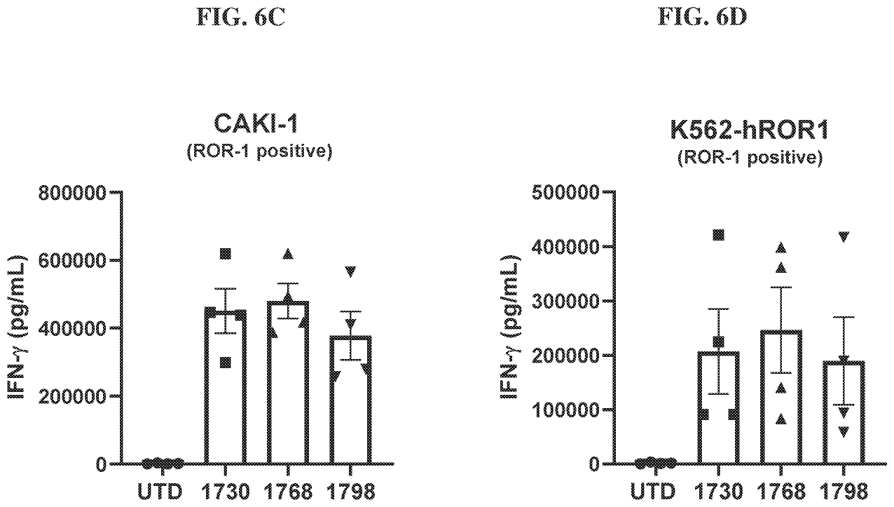

Cell supernatants were collected and frozen until further use. Cytokine analysis for interferon gamma (IFN-γ, see FIGS. 6A-6D) and IL-2 was performed using Meso Scale Discovery kits (Cat #K151TTK & K151TVK; MSD). High IFN-γ release was observed versus all targeted tumor cell lines expressing ROR1 without significant differences between the tested CAR constructs (CAR only (1730), CAR and GOT2 (1768) or CAR, GOT2 and TIGAR (1798), see FIGS. 6B to 6D). In turn untransduced control (UTD) cells did not show any IFN-γ release after co-incubation with ROR1 expressing tumor cell lines (see FIG. 6A).

Next, in vitro T cell cytolytic activity was assessed. Tumor cells (A549 cell line; NSLC-endogenously expressing ROR1) were transduced with red fluorescent NucLightRed lentiviral vector (Cat #4717; Sartorius) and selected for viral integration with puromycin. 20,000 cells/well engineered tumor cell lines were plated in a 96-well clear bottom plate for 16 h at 37° C. CAR-T cells (untransduced (UTD). CAR only (1730), CAR and GOT2 (1768) or CAR, GOT2 and TIGAR (1798)) were stimulated with plate-bound ROR1 antigen four times for 3 days per stimulation prior to co-culture with the plated tumor cells at effector to T cell ratios (E:T::1:1) for 132 h (5.5 days). Cytotoxicity was assessed by enumerating the cell count (red signal).

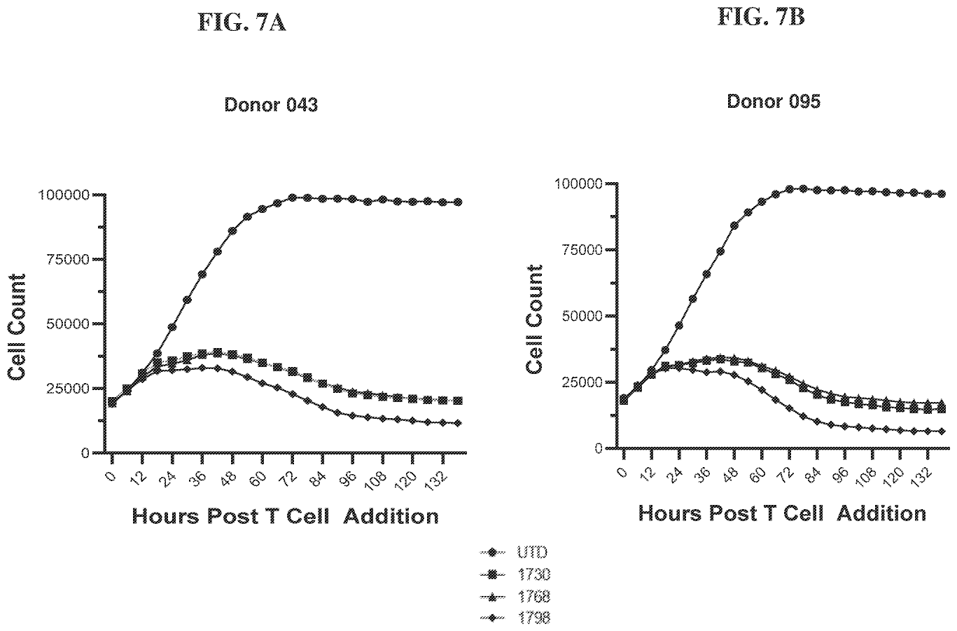

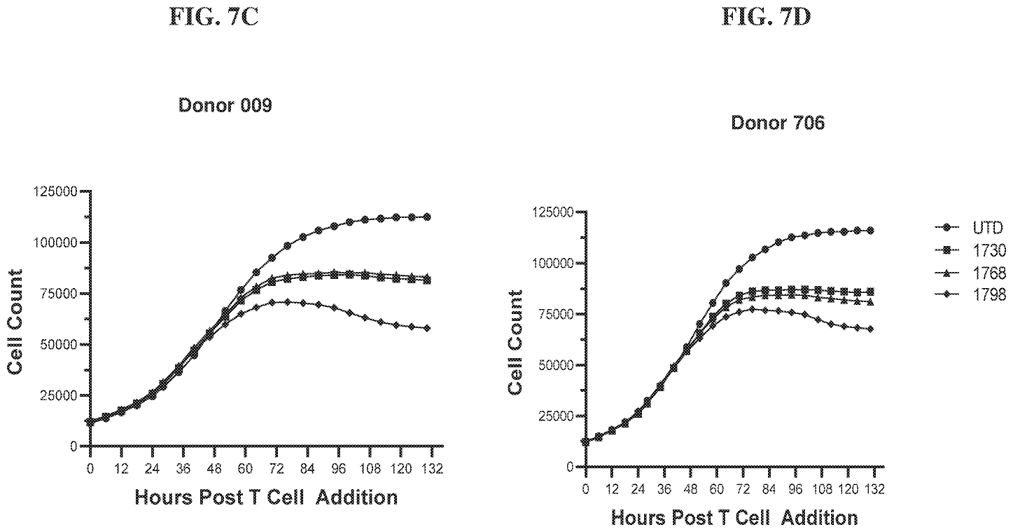

We observed cell killing in all three conditions with the ROR1 targeting scFv (clone 1730). We further observed enhanced killing when we introduced an additional transgene encoding a metabolism modulating polypeptide, i.e. GOT2 (compare clones 1730 vs 1768) but surprisingly we consistently observed even more pronounced killing when we expressed two metabolism modulating polypeptides, namely GOT2 and TIGAR (1798) in four donors (see FIGS. 7A to 7D).

### Example 19: Impact of Expressing at Least Two Exemplary Metabolism Modulating Polypeptides on Immune Cell Function Expressing a CAR Polypeptide In Vivo

CAKI-1 (human clear cell renal cell carcinoma) xenografts were established in female NOD-SCID-IL2Ry null (NSG) mice by subcutaneous injection in the right flank with 5×106 cells suspended in 0.1 mL of 50% Matrigel/serum-free RPMI 1640 culture media. Mice were randomized into treatment groups of 5 mice each based on tumor volume (117±25 mm³ on day 20 post implantation). Tumor volume was measured using calipers and calculated using the formula (L×W×H)/2 mm³. Animals received a single intravenous administration of ROR1-targeted CAR-Ts on Day 21 via intravenous tail vein injection. (indicated by dashed vertical line) post tumor implantation (study day 0) at a dose of 5×10⁶ CAR only (1730) or CAR and GOT2 (1768) or CAR. GOT2 and TIGAR (1798) T cells. Control animals were left untreated. Tumor volume and body weights were monitored twice weekly until 60 days. Mice were euthanized when tumor volumes reached 1000 mm³, or in the event of tumor ulceration.

In order to compare CAR only (1730), CAR and GOT2 (1768) or CAR, GOT2 and TIGAR (1798), a stringent xenograft model system was developed using, CAKI-1 tumor cells. All animals tolerated the single dose of 5×10⁶ tumor cells well. A serious of in vivo tests were conducted with T cells from two donors (Donor 1 and Donor 2), see Table 18. Initially, CAR Ts expressing the anti-ROR1 CAR and either the metabolism modifying polypeptide GOT2 or TIGAR were compared to controls at Day 46 for Donor 1. Whereas both CAR Ts expressing a benchmark CAR or only the anti-ROR1 CAR did not show marked activity, both GOT2 and TIGAR co-expression with the anti-ROR1 CAR were showing anti-tumor efficacy including partial responses, but no complete responses. GOT2 was slightly better than TIGAR. See section A) in Table 18.

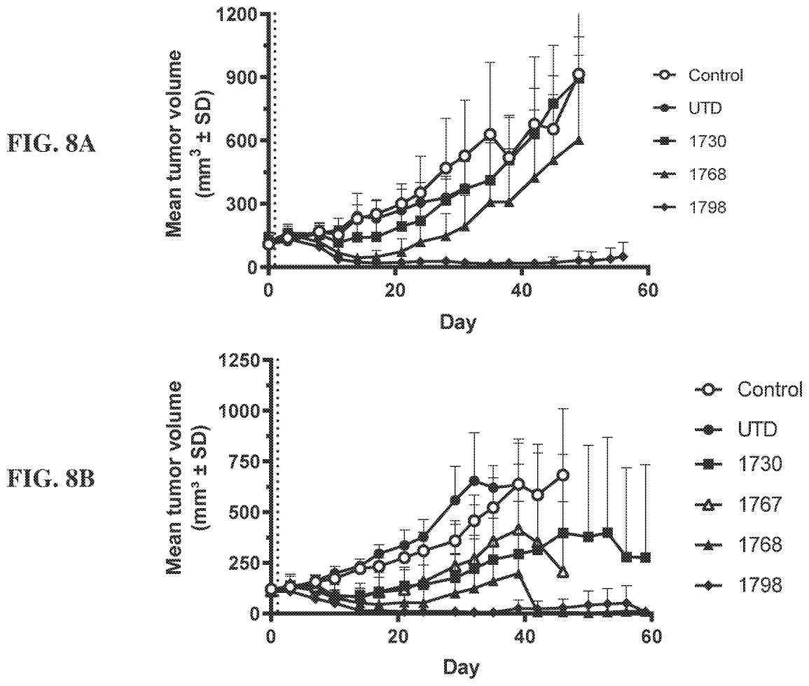

The experiment was repeated with T cells from the same donor comparing GOT2 coexpression with GOT2 and TIGAR co-expression resulting in a clearly increased anti-tumor activity of the combination, e.g., leading to 100% PRs (vs. 80% for GOT2 only) and 60% CR (vs. 0% for GOT2 only). See section B) in Table 18. Mean tumor volumes of this experiment are shown in FIG. 8A.

In a third experiment using different donor (Donor 2), the combination of GOT2 and TIGAR was compared to the anti-ROR1 CAR having some minor activity (e.g., 40% PRs), whereas co-expression of TIGAR improved the activity and lead to 40% CRs, whereas both GOT2 as well as GOT2 and TIGAR expression lead to high activity with 80% PR and 80/40% CRs. See section C) in Table 18. Mean tumor volumes of this experiment are shown in FIG. 8B.

TABLE 18
Anti-tumor activity in CAKI xenografts

| | | | | | |
|---|---|---|---|---|---|
| | | | partial | complete | |
| | metab. mod. | | responses | responses | |
| Construct | polypeptide | T/C (%) | (%) | (%) | Activity |

A) Donor 1

| | | | | | |
|---|---|---|---|---|---|
| Benchmark | −/− | 110 | 0 | 0 | (−) |
| 1730 | −/− | 49 | 0 | 0 | (−) |
| 1767 | TIGAR | 14 | 40 | 0 | (+) |
| 1768 | GOT2 | 2 | 60 | 0 | (++) |

B) Donor 1

| | | | | | |
|---|---|---|---|---|---|
| 1730 | −/− | 108 | 0 | 0 | (−) |
| 1768 | GOT2 | 79 | 80 | 0 | (+) |
| 1798 | GOT2 + TIGAR | 2 | 100 | 60 | (+++) |

C) Donor 2

| | | | | | |
|---|---|---|---|---|---|
| 1730 | −/− | 42 | 40 | 0 | (+) |
| 1767 | TIGAR | 59 | 40 | 40 | (++) |
| 1768 | GOT2 | 1 | 80 | 80 | (+++) |
| 1798 | GOT2 + TIGAR | 1 | 80 | 40 | (+++) |

Benchmark: CAR against different tumor target not expressed; T/C: tumor growth inhibition calculated from median tumor volumes of treated (T) relative to untreated (C) at the control group endpoint according to formula
$\frac{T}{C} ( \% ) = \frac{Median tumor volume ( Treated ) \times 100}{Median tumor volume ( Control )} .$

Similar results were obtained with the dose level of 2.5×10⁶ CAR T cells.

In summary, despite inter-donor variabilities CAR Ts expressing two exemplary metabolism modifying polypeptides (here GOT2 and TIGAR) in addition to the CAR lead to robust anti-tumor responses with 80% or 100% animals exhibiting a partial responses and 40% to 60% complete responses. Apparently, T cells from Donor 2 were more active, showing already activity for the CAR, therefore being less discriminative for the different settings of co-expressing metabolism modifying polypeptides.

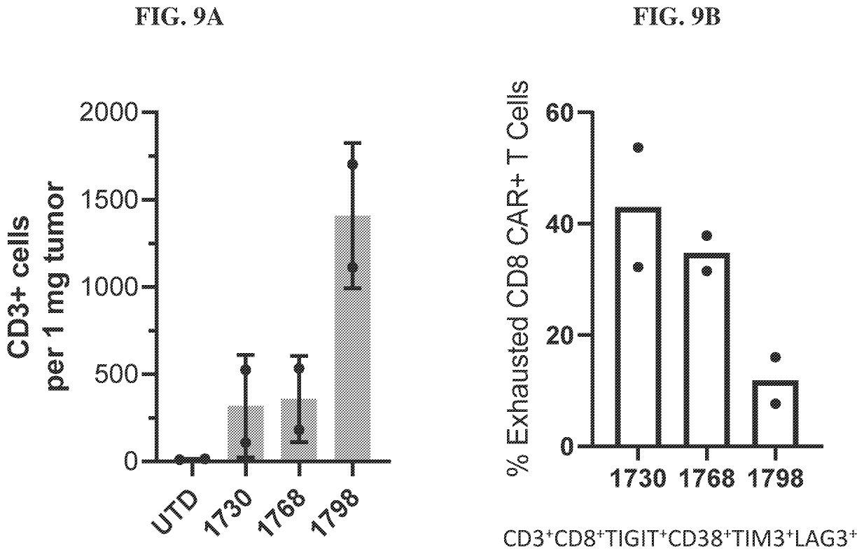

Next, the in vivo activity of the CAR Ts within the tumor itself was assessed by analyzing tumor infiltrating lymphocytes (TILs as CD3⁺ cells) ex vivo. Two mice were sacrificed on day 7 and tumor xenografts were collected and weighed. Tumors were processed to a single cell suspension using the Miltenyi GentleMACS tissue Dissociator (Cat #130-095-929; Miltenyi). Cells were stained for CD3⁺ expression and assessed by flow cytometry as detailed above (see Example 17). CAR Ts expressing the two metabolism modifying polypeptides GOT2 and TIGAR (1798) exhibited a significantly higher infiltration of T cells into the tumor compared to moderately increased infiltration for CAR Ts without or with one metabolism modifying polypeptide (1730, and 1768 co-expressing GOT2). See FIG. 9A.

At the same time CD8⁺ CAR Ts expressing both GOT2 and TIGIT, when stained for exhaustion markers CD38, TIGIT, LAG3 and TIM3, showed a substantially reduced exhaustion phenotype compared to CAR Ts expressing no (1730) or only one metabolism modifying polypeptide (GOT2, 1768) indicating that the observed increased numbers of CAR T cells in the tumor were not exhausted, but active. See FIG. 9B.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either”, “one of”, “only one of”, or “exactly one of”. “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A genetically engineered immune cell, which
(i) expresses or overly expresses at least two metabolism modulating polypeptides selected from the group consisting of Glutamic-oxaloacetic transaminase 2 (GOT2), Glucose transporter 1 (GLUT1), Lactate dehydrogenase A (LDHA), Pyruvate dehydrogenase kinase 1 (PDK1), TP53-inducible glycolysis and apoptosis regulator (TIGAR), Cystathionine gamma-lyase (CTH), Argininosuccinate synthase 1 (ASS1) and Phosphoserine phosphatase (PSPH); and
(ii) expresses a chimeric receptor polypeptide; wherein the chimeric receptor polypeptide comprises:
(a) an extracellular target binding domain;
(b) a transmembrane domain; and
(c) at least one cytoplasmic signaling domain.

2. The genetically engineered immune cell of claim 1, wherein the two metabolism modulating polypeptides are selected from the group consisting of:
(a) GOT2 and TIGAR;
(b) GOT2 and GLUT1;
(c) GOT2 and PDK1;
(d) TIGAR and GLUT1;
(e) PDK1 and CTH;
(f) CTH and PSPH;
(g) GLUT1 and ASS1; and
(h) GLUT1 and PSPH.

3. The genetically engineered immune cell of claim 1, wherein the chimeric receptor polypeptide comprises one or more of the following features:
(i) the chimeric receptor polypeptide further comprises a signal peptide at its N-terminus;
(ii) the chimeric receptor polypeptide further comprises a hinge domain, which is located at the C-terminus of (a) and the N-terminus of (b);
(iii) the chimeric receptor polypeptide is free of a hinge domain;
(iv) the chimeric receptor polypeptide further comprises at least one costimulatory signaling domain;
(v) the chimeric receptor polypeptide is free of a co-stimulatory signaling domain; and
(vi) the cytoplasmic signaling domain comprises an immunoreceptor tyrosine-based activation motif (ITAM).

4. The genetically engineered immune cell of claim 1, wherein the chimeric receptor polypeptide is a chimeric antigen receptor (CAR) polypeptide, in which (ii) (a) is an extracellular antigen binding domain.

5. The genetically engineered immune cell of claim 4, wherein the extracellular antigen binding domain is a single chain variable fragment (scFv) or a single domain antibody that binds to a tumor antigen, a pathogenic antigen, or an immune cell specific to an autoantigen.

6. The genetically engineered immune cell of claim 5, wherein the extracellular antigen binding domain binds to the tumor antigen, which is associated with a solid tumor.

7. The genetically engineered immune cell of claim 5, wherein the extracellular antigen binding domain binds to the pathogenic antigen, which is a bacterial antigen, a viral antigen, or a fungal antigen.

8. The genetically engineered immune cell of claim 1, wherein the transmembrane domain is of a membrane protein selected from the group consisting of CD8a, CD8b, 4-1BB, CD28, CD34, CD4, FceRIg, CD16A, OX40, CD3z, CD3e, CD3g, CD3d, TCRa, CD32, CD64, VEGFR2, FAS, FGFR2B, DNAM1, 2B4, NKG2D, NKp44 and NKp46.

9. The genetically engineered immune cell of claim 3, wherein the at least one co-stimulatory signaling domain (iv) is of a costimulatory molecule selected from the group consisting of 4-1BB, CD28, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL.

10. The genetically engineered immune cell of claim 3, wherein the at least one co-stimulatory signaling domain is a CD28 costimulatory signaling domain or a 4-1BB co-stimulatory signaling domain.

11. The genetically engineered immune cell of claim 3, wherein the chimeric receptor polypeptide comprises at least two costimulatory signaling domains.

12. The genetically engineered immune cell of claim 11, wherein
(i) one of the co-stimulatory signaling domains is a CD28 co-stimulatory signaling domain; and the other co-stimulatory domain is selected from the group consisting of a CD8α, 4-1BB, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL co-stimulatory signaling domain;
(ii) one of the co-stimulatory signaling domains is a CD8α co-stimulatory signaling domain; and the other co-stimulatory domain is selected from the group consisting of a CD28, 4-1BB, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL co-stimulatory signaling domain; or
(iii) one of the co-stimulatory signaling domains is a 4-1BB co-stimulatory signaling domain; and the other co-stimulatory domain is selected from the group consisting of a CD8α, CD28, 2B4, OX40, OX40L, ICOS, CD27, GITR, HVEM, TIM1, LFA1, CD2, DAP10, DAP12, DNAM-1, NKG2D, NKp30, NKp44, NKp46 and JAMAL co-stimulatory signaling domain.

13. The genetically engineered immune cell of claim 1, wherein the cytoplasmic signaling domain of (c) is a cytoplasmic domain of CD3ζ or FcεR1γ, preferably CD3ζ.

14. The genetically engineered immune cell of claim 3, wherein the hinge domain (ii) is a hinge domain of CD8α, CD28 or IgG, preferably CD8α or CD28.

15. The genetically engineered immune cell of claim 1, wherein the immune cell is an αβ T cell, a γδ T cell, or a natural killer (NK) cell.

16. The genetically engineered immune cell of claim 4, wherein the chimeric receptor polypeptide is a CAR polypeptide, which comprises:
(i) a CD28 co-stimulatory domain, a CD28 transmembrane domain and a CD28 hinge domain;
(ii) a 4-1BB co-stimulatory domain, a CD8α transmembrane domain and a CD8α hinge domain.

17. The genetically engineered immune cell of claim 1, wherein
(i) the immune cell is derived from cell lines; or
(ii) the immune cell is derived from peripheral blood mononuclear cells (PBMC), hematopoietic stem cells (HSCs), cord blood stem cells or induced pluripotent stem cells (iPSCs).

18. The genetically engineered immune cell of claim 1, wherein the immune cell comprises a nucleic acid or nucleic acid set, which collectively comprises:
(A) a first nucleotide sequence encoding one of the at least two metabolism modulating polypeptides of (i);
(B) a second nucleotide sequence encoding the other one of the at least two metabolism modulating polypeptides (i); and
(C) a third nucleotide sequence encoding the chimeric receptor polypeptide of (ii).

19. The genetically engineered immune cell of claim 18, wherein the nucleic acid further comprises a fourth and a fifth nucleotide sequence located, wherein the fourth nucleotide sequence is located between the first nucleotide sequence and the second nucleotide sequence, wherein the fifth nucleotide sequence is located between the second nucleotide sequence and the third nucleotide sequence, wherein the fourth and the fifth nucleotide sequence encodes a ribosomal skipping site, an internal ribosome entry site (IRES), or a promoter.

20. The genetically engineered immune cell of claim 18, wherein the nucleic acid or nucleic acid set is comprised within one or more viral vectors.

21. A pharmaceutical composition, comprising the genetically engineered immune cell of claim 1.

22. (canceled)

23. A method for inhibiting and/or killing cells expressing a target antigen in a subject, the method comprising administering to a subject in need thereof a population of the genetically engineered immune cells set forth in claim 1, or a pharmaceutical composition comprising the population of the genetically engineered immune cells.

24. The method of claim 23, wherein the subject is a human patient suffering from a cancer and the target antigen is a tumor antigen of a solid tumor; optionally
wherein the cancer is selected from the group consisting of carcinoma, lymphoma, sarcoma and blastoma, or
wherein the cancer is selected from the group consisting of a cancer of B-cell origin, breast cancer, gastric cancer, neuroblastoma, osteosarcoma, lung cancer, skin cancer, prostate cancer, colon cancer, renal cell carcinoma, ovarian cancer, rhabdomyosarcoma, mesothelioma, pancreatic cancer, head and neck cancer, retinoblastoma, glioma, glioblastoma, liver cancer, and thyroid cancer; optionally wherein the cancer of B-cell origin is selected from the group consisting of Hodgkin lymphoma and non-Hodgkin lymphoma.

25. The method of claim 23, wherein at least some of the cells expressing the target antigen are located in a low-glucose environment.

26. The method of claim 23, wherein the immune cells meet one or more of the following:
(i) the immune cells are autologous;
(ii) the immune cells are allogeneic;
(iii) the immune cells are activated, expanded, or both ex vivo, and
(iv) the immune cells are activated in the presence of one or more of 4-1BB ligand, anti-4-1BB antibody, IL-15, anti-IL-15 receptor antibody, IL-2, IL-2/IL-15Rβγ superagonist, IL-12, IL-21 and K562 cells, and an engineered artificial stimulatory cell or particle.

27. A nucleic acid or nucleic acid set, which collectively comprises:
(A) a first nucleotide sequence encoding the one of the at least two metabolism modulating polypeptides (i) of claim 1;
(B) a second nucleotide sequence encoding the other of the at least two metabolism modulating polypeptides (i) of claim 1; and
(C) a third nucleotide sequence encoding the chimeric receptor polypeptide set forth in claim 1.

28. A vector or vector set comprising the nucleic acid or nucleic acid set of claim 27.

29. The vector or vector set of claim 28, wherein the vector(s) is a viral vector, preferably a lentiviral or retroviral vector.

30. A method of producing viral particles, wherein
(a) providing host cells stably transfected with the nucleic acid or nucleic acid set of claim 27 or a vector or vector set comprising the nucleic acid or nucleic acid set;
(b) growing the stably transfected host cells in a cell culture medium under conditions allowing for producing viral particles by the host cells; and
(c) harvesting the viral particles from the cell culture medium.

31. A viral particle produced according to the method of claim 30.

32. A method of producing an immune cell that expresses the metabolism modulating polypeptides and the chimeric receptor polypeptides set forth in claim 1, the method comprising incubating immune cells with a viral particle comprising a nucleic acid or nucleic acid set, under conditions allowing for infection of immune cells by the viral particle;
wherein the nucleic acid or nucleic acid set comprises or collectively comprises:
(A) a first nucleotide sequence encoding the one of the at least two metabolism modulating polypeptides (i) of claim 1;
(B) a second nucleotide sequence encoding the other of the at least two metabolism modulating polypeptides (i) of claim 1; and
(C) a third nucleotide sequence encoding the chimeric receptor polypeptide set forth in claim 1.

33. An immune cell produced by the method of claim 32.

34. A method of modifying the metabolism of immune cells, comprising transfecting immune cells transiently or stably with the vector or vector set of claim 28; and collecting the immune cells transfected with the vector or vector set.

35. A method for generating modified immune cells in vivo, the method comprising administering to a subject in need thereof the nucleic acid or nucleic acid set of claim 27; a vector or vector set comprising the nucleic acid or nucleic acid set, or viral-particles comprising the nucleic acid or nucleic acid set.
